SEQUENCE LISTING (91 sequences)

SEQ ID NO: 1 - DNA, 1683 bp, Homo sapiens
atggagacttattccttgtctttgggtaatcaatcagtggtggaacctaacatagcaatacagtcagcaaatttctcttcagaaaatgcggtggggccttcaaatgttcgcttctctgtgcagaaaggagctagcagttctctagtttctagttcaacatttatacatacaaatgtggatggccttaacccagatgcacagactgagcttcaggtcttgcttaatatgacgaaaaattacaccaagacatgcggctttgtagtttatcaaaatgacaagcttttccaatcaaaaacttttacagctaaatcggattttagtcaaaaaattatctcaagcaaaactgatgaaaatgagcaagatcagagtgcttctgttgacatggtctttagtccaaagtacaaccaaaaagaatttcaactctattcctatgcctgtgtctattggaatttgtcagcgaaggactgggacacatatggctgtcaaaaagacaagggcactgatggattcctgcgctgccgctgcaaccatactactaattttgctgtattaatgactttcaaaaaggattatcaatatcccaaatcacttgacatattatccaacgttggatgtgcactgtctgttactggtctggctctcacagttatatttcagattgtcaccaggaaagtcagaaaaacctcagtaacctgggttttggtcaatctgtgcatatcaatgttgattttcaacctcctctttgtgtttggaattgaaaactccaataagaacttgcagacaagtgatggtgacatcaataatattgactttgacaataatgacatacccaggacagacaccattaacatcccgaatcccatgtgcactgcgattgccgccttactgcactattttctgttagtgacatttacctggaacgcactcagcgctgcacagctctattaccttctaataaggaccatgaagcctcttcctcggcatttcattcttttcatctcattaattggatggggagtcccagctatagtagtggctataacagtgggagttatttattctcagaatggaaataatccacagtgggaattagactaccggcaagagaaaatctgctggctggcaattccagaacccaatggtgttataaaaagtccgctgttgtggtcattcatcgtacctgtaaccattatcctcatcagcaatgttgttatgtttattacaatctcgatcaaagtgctgtggaagaataaccagaacctgacaagcacaaaaaaagtttcatccatgaagaagattgttagcacattatctgttgcagttgtttttggaattacctggattctagcatacctgatgctagttaatgatgatagcatcaggatcgtcttcagctacatattctgccttttcaacactacacagggattgcaaatttttatcctgtacactgttagaacaaaagtcttccagagtgaagcttccaaagtgttgatgttgctatcgtctattgggagaaggaagtcattgccttcagtgacgcggccgaggctgcgtgtaaagatgtataatttcctcaggtcattgccaaccttacatgaacgctttaggctactggaaacctctccgagtactgaggaaatcacactctctgaaagtgacaatgcaaaggaaagcatctag

SEQ ID NO: 2 - protein, 560 aa, Homo sapiens
METYSLSLGNQSVVEPNIAIQSANFSSENAVGPSNVRFSVQKGASSSLVSSSTFIHTNVDGLNPDAQTELQVLLNMTKNYTKTCGFVVYQNDKLFQSKTFTAKSDFSQKIISSKTDENEQDQSASVDMVFSPKYNQKEFQLYSYACVYWNLSAKDWDTYGCQKDKGTDGFLRCRCNHTTNFAVLMTFKKDYQYPKSLDILSNVGCALSVTGLALTVIFQIVTRKVRKTSVTWVLVNLCISMLIFNLLFVFGIENSNKNLQTSDGDINNIDFDNNDIPRTDTINIPNPMCTAIAALLHYFLLVTFTWNALSAAQLYYLLIRTMKPLPRHFILFISLIGWGVPAIVVAITVGVIYSQNGNNPQWELDYRQEKICWLAIPEPNGVIKSPLLWSFIVPVTIILISNVVMFITISIKVLWKNNQNLTSTKKVSSMKKIVSTLSVAVVFGITWILAYLMLVNDDSIRIVFSYIFCLFNTTQGLQIFILYTVRTKVFQSEASKVLMLLSSIGRRKSLPSVTRPRLRVKMYNFLRSLPTLHERFRLLETSPSTEEITLSESDNAKESI

SEQ ID NO: 3 - DNA, 2212 bp, Homo sapiens
ccacgctttccctccctgaccacaggtgatccgcctgcctcagcctcccgaagtgcagggattacaggcgtagtaagtaagccaccacacctggccgccactcttatttttaaaagttgacatcagtttgtgaaaaaggactgttgtttcatcaaatttcagcaaatgatgatcaatagcacattaaaaatggcttcatctttgtggaagttttgactggatatagatccctgacatttgagaccaaaggaaagcctcttgatggtgtaactggaccagaatgaagagaaagaaactattatcaaagacccttggaaacaggaaactccaaacctgatgcgggtctcagggcagtatctatgagcaggtgaaatagaaagtacatctaactagatgttttttcatgcagattaaattattttgaccaaagttgtacccaaatgcacatgcatggaagagctaacactaggggacaagcaagggggaggaagaggaaaccaacctttatgtacagcctttcatgtgcctggcatgttgcatatgttatcacatttaatccttataaaacttctgtgagttgaatgttattcccatattataaataattatagccaataacacttactaattgttgagcacctactgcatgccaaatattgtgccaaatattaatgtatttattagtttatcatatttaatttttataacaccataaataggtattaatgtacacattttatagatgaggaaaatgtggttctgagaggtgaagcattttgcctagtgatcacagctaaaaagtgatagagctgttctttattttaaagttcacattgtactaccctggctccctaatcacagatgggcagggtaggggttgggtggggacagaagttggagagtggatgtggctgccaaccacacaagttgtgccaacccacagattgaggaaagatgctaaatttggaatctggcaaaccagtgtttggttcttagctctgccacttctaagctgtgtgaaacttggttgaggtccctaacttctcctgagggtgaacaactcacaaagttgttttgcttattaaatgtgataacacctgtaaacatctaacagagtgcctagcacatagcagggatctagcaattgaattagggttatttgtttctgtctactgattgggtattgtttctgacacttacccaagtgtgaatagcctataacactggtataatttgtgaaatgatgctgccatctagtgaaaaccaagacacacacacacacacacacacacacacacatacacacacacgtgcgcgcgcatggacacccagcttcaccaatgacaatatggattggcatgttttagcctcacaacacagagccctggggctaactggcacctagagaggtcatctcggccagtgccttccaaactaccagtgctgaaaagccagttcaaaaaattttgaacccattgcacaccaatatttttgtgaaataccataaaaataaattactggaaaaatgaaataaaaaatatgtataaaatacaaaccaaaattttagaactgttagattcaacagcaaaaaattgctgtatacatctctgaccaattgctttcagtttctgtgcttatctctctacgacctttgtaacacacagtgaaccagcgctggcccatggatacactctagtagccccaatctagctaaggcagccccttatagttaatcaatcctgtcaaacaggaaaggctggcaaaaccactggtctgcatgtactttgtcctttacacaaggaaggatgcaaacgtggaaaactgagtggacatggtgttcaggagattgaggctcagctaaattccagcttatttacctgcagttgcttacaaagtgtttggacataattgtgtaaagctagggttttttttctggtttttaaaacaggtaaaggatgtcacagcaccacttaataacatttcttctgaagtccagattttaacatctgatgccaataaattaactgctgagaacatcactagtgctacgcgagtggttggacagatattcaacacttccagaaatgcttcacctgaggcaaagaaagttgccatagtaacagtgagtcaactcctagatgccagtgaagatgcttttcaaagagttgctgctactgctaatgatgatgcccttacaacgcttattgagcaa

SEQ ID NO: 4 - DNA, 449 bp, Homo sapiens
acagtaaaacttacctgttgtggtctttttaatcacctcgtttgagttttatctgtttctctcctttatttcccagtcctctcagaaagtcttcctcaatgtattttgctcaggattaagaattagataaaacctgttgtttattattattcggcataatggacttggtagtttttctatttttcaatagatttgtacttgaataaggtgaagaatttcacacaacatacaagagtaccattgttccttatatcgttaaatctttgtgacacactttgacaaaaatgtagaacctataacaaattcttttacaagttactataaaggacacaaagagaaaactttaccttccagaacaaaatgactcctgatgaacagtgtgtggggatttgcttgtatgtattaaacttttgacctctgaaaaaaaaaaaaaaaaaaaaaaaaaaaag

SEQ ID NO: 5 - DNA, 80 bp, Artificial Sequence
  Synthesized Oligonucleotide.
gctgtgcagcgctgagtgcgttccaggtaaatgtcactaacagaaaatagtgcagtaaggcggcaatcgcagtgcacatg

SEQ ID NO: 6 - DNA, 23 bp, Homo sapiens
cagacaccattaacatcccgaat

SEQ ID NO: 7 - DNA, 22 bp, Homo sapiens
agaatgaaatgccgaggaagag

SEQ ID NO: 8 - protein, 1230 aa, Rat
MCPPQLFILMMLLAPVVHAFSRAPIPMAVVRRELSCESYPIELRCPGTDVIMIESANYGRTDDKICDSDPAQMENIRCYLPDAYKIMSQRCNNRTQCAVVAGPDVFPDPCPGTYKYLEVQYECVPYKVEQKVFLCPGLLKGVYQSEHLFESDHQSGAWCKDPLQASDKIYYMPWTPYRTDTLTEYSSKDDFIAGRPTTTYKLPHRVDGTGFVVYDGALFFNKERTRNIVKFDLRTRIKSGEAIIANANYHDTSPYRWGGKSDIDLAVDENGLWVIYATEQNNGKIVISQLNPYTLRIEGTWDTAYDKRSASNAFMICGILYVVKSVYEDDDNEATGNKIDYIYNTDQSKDSLVDVPFPNSYQYIAAVDYNPRDNLLYVWNNYHVVKYSLDFGPLDSRSGPVHHGQVSYISPPIHLDSDLERPPVRGISTTGPLGMGSTTTSTTLRTTTWNLGRSTTPSLPGRRNRSTSTPSPAIEVLDVTTHLPSAASQIPAMEESCEAVEAREIMWFKTRQGQVAKQSCPAGTIGVSTYLCLAPDGIWDPQGPDLSNCSSPWVNHITQKLKSGETAANIARELAEQTRNHLNAGDITYSVRAMDQLVGLLDVQLRNLTPGGKDSAARSLNKLQKRERSCRAYVQAMVETVNNLLQPQALNAWRDLTTSDQLRAATMLLDTVEESAFVLADNLLKTDIVRENTDNIQLEVARLSTEGNLEDLKFPENTGHGSTIQLSANTLKQNGRNGEIRVAFVLYNNLGPYLSTENASMKLGTEAMSTNHSVIVNSPVITAAINKEFSNKVYLADPVVFTVKHIKQSEENFNPNCSFWSYSKRTMTGYWSTQGCRLLTTNKTHTTCSCNHLTNFAVLMAHVEVKHSDAVHDLLLDVITWVGILLSLVCLLICIFTFCFFRGLQSDRNTIHKNLCISLFVAELLFLIGINRTDQPIACAVFAALLHFFFLAAFTWMFLEGVQLYIMLVEVFESEHSRRKYFYLVGYGMPALIVAVSAAVDYRSYGTDKVCWLRLDTYFIWSFIGPATLIIMLNVIFLGIALYKMFHHTAILKPESGCLDNIKSWVIGAIALLCLLGLTWAFGLMYINESTVIMAYLFTIFNSLQGMFIFIFHCVLQKKVRKEYGKCLRTHCCSGKSTESSIGSGKTSGSRTPGRYSTGSQSRIRRMWNDTVRKQSESSFITGDINSSASLNREPYRETSMGVKLNIAYQIGASEQCQGYKCHGYSTTEW

SEQ ID NO: 9 - protein, 1527 aa, Rat
MCPPQLFILMMLLAPVVHGGKHNERHPALAAPLRHAEHSPGGPLPPRHLLQQPAAERSTAHRGQGPRGTARGVRGPGAPGAQIAAQAFSRAPIPMAVVRRELSCESYPIELRCPGTDVIMIESANYGRTDDKICDSDPAQMENIRCYLPDAYKIMSQRCNNRTQCAVVAGPDVFPDPCPGTYKYLEVQYECVPYKVEQKVFLCPGLLKGVYQSEHLFESDHQSGAWCKDPLQASDKIYYMPWTPYRTDTLTEYSSKDDFIAGRPTTTYKLPHRVDGTGFVVYDGALFFNKERTRNIVKFDLRTRIKSGEAIIANANYHDTSPYRWGGKSDIDLAVDENGLWVIYATEQNNGKIVISQLNPYTLRIEGTWDTAYDKRSASNAFMICGILYVVKSVYEDDDNEATGNKIDYIYNTDQSKDSLVDVPFPNSYQYIAAVDYNPRDNLLYVWNNYHVVKYSLDFGPLDSRSGPVHHGQVSYISPPIHLDSDLERPPVRGISTTGPLGMGSTTTSTTLRTTTWNLGRSTTPSLPGRRNRSTSTPSPAIEVLDVTTHLPSAASQIPAMEESCEAVEAREIMWFKTRQGQVAKQSCPAGTIGVSTYLCLAPDGIWDPQGPDLSNCSSPWVNHITQKLKSGETAANIARELAEQTRNHLNAGDITYSVRAMDQLVGLLDVQLRNLTPGGKDSAARSLNKLQKRERSCRAYVQAMVETVNNLLQPQALNAWRDLTTSDQLRAATMLLDTVEESAFVLADNLLKTDIVRENTDNIQLEVARLSTEGNLEDLKFPENTGHGSTIQLSANTLKQNGRNGEIRVAFVLYNNLGPYLSTENASMKLGTEAMSTNHSVIVNSPVITAAINKEFSNKVYLADPVVFTVKHIKQSEENFNPNCSFWSYSKRTMTGYWSTQGCRLLTTNKTHTTCSCNHLTNFAVLMAHVEVKHSDAVHDLLLDVITWVGILLSLVCLLICIFTFCFFRGLQSDRNTIHKNLCISLFVAELLFLIGINRTDQPIACAVFAALLHFFFLAAFTWMFLEGVQLYIMLVEVFESEHSRRKYFYLVGYGMPALIVAVSAAVDYRSYGTDKVCWLRLDTYFIWSFIGPATLIIMLNVIFLGIALYKMFHHTAILKPESGCLDNIKSWVIGAIALLCLLGLTWAFGLMYINESTVIMAYLFTIFNSLQGMFIFIFHCVLQKKVRKEYGKCLRTHCCSGKSTESSIGSGKTSGSRTPGRYSTGSQSRIRRMWNDTVRKQSESSFITGDINSSASLNREGLLNNARDTSVMDTLPLNGNHGNSYSIAGGEYLSNCVQIIDRGYNHNETALEKKILKELTSNYIPSYLNNHERSSEQNRNMMNKLVDNLGSGSEDDAIVLDDAASFNHEESLGLELIHEESDAPLLPPRVYSTDNHQPHHYSRRRLPQDHSESFFPLLTDEHTEDPQSPHRDSLYTSMPALAGVPAADSVTTSTQTEAAAAKGGDAEDVYYKSMPNLGSRNHVHPLHAYYQLGRGSSDGFIVPPNKDGASPEGTSKGPAHLVTSL

SEQ ID NO: 10 - protein, 541 aa, Homo sapiens
MDFESGQVDPLASVILPPNLLENLSPEDSVLVRRAQFTFFNKTGLFQDVGPQRKTLVSYVMACSIGNITIQNLKDPVQIKIKHTRTQEVHHPICAFWDLNKNKSFGGWNTSGCVAHRDSDASETVCLCNHFTHFGVLMDLPRSASQLDARNTKVLTFISYIGCGISAIFSAATLLTYVAFEKLRRDYPSKILMNLSTALLFLNLLFLLDGWITSFNVDGLCIAVAVLLHFFLLATFTWMGLEAIHMYIALVKVFNTYIRRYILKFCIIGWGLPALVVSVVLASRNNNEVYGKESYGKEKGDEFCWIQDPVIFYVTCAGYFGVMFFLNIAMFIVVMVQICGRNGKRSNRTLREEVLRNLRSVVSLTFLLGMTWGFAFFAWGPLNIPFMYLFSIFNSLQGLFIFIFHCAMKENVQKQWRQHLCCGRFRLADNSDWSKTATNIIKKSSDNLGKSLSSSSIGSNSTYLTSKSKSSSTTYFKRNSHTDSASMDKSLSKLAHADGDQTSIIPVHQVIDKVKGYCNAHSDNFYKNIIMSDTFSHSTKF

SEQ ID NO: 11 - protein, 1582 aa, Caenorhabditis elegans
MATASTEISEFSEAIESTFDLDFTAHQTEIIGTYWNLRALLRLHRSLVAIDHVSQKSFWERYNHWIQLSMLVSNQNVNLCQSNICQNGGTCLVASSVPATATCPKNSIYYMGSCYVFDTTLRNWNDAALYCNNMNSATLPLVESAEDQAFFAGYLQAMIPSNPPADMRPPPDGIWTAVRGVNNVTRASWVYYPGSFLVTDTFWAPQEPNIYVNYNDVCVALQSDSFYREWTTALCTILKYTVCKVAPTQIQAKYVAQCSCPNGYGGQTCETQSTTNQQASTQRTCGSNDFQFSCPNDQTITVDFASFGAQGGSIITSPPDALLQQIVQKVNAETKKTVNFWIGTPNNCQLLMVTGSSTSYSQCPSSPSSTANVICSTVPQSTASVSARPTQSAPVDPVSQTMARREVYTGVQPIASALGGQSKKTNRKLNNICQTKIGAPLSLFLFSRNEVITGFVCISLISASPQIIYYLCAVSLICHPSVPDSINKPRYCKKEKKDGITYEQTRACMLHEQPCPDPQNVEGTVTRYCNCQTAKWETPDTTNCTHRWVAEMETAIKDNQPVEDISSTVNRQLKSTIERTLFGGDITGTVRLSNDMLSLARNQFSVLNDRNLRENKARNFTENLGGSGDQLLSPVAATVWDQLSSTIRIQHASKLMSVLEQSVLLLGDYMTDQKLNLQYINWAMEVERSEPEVQTFGAAASPNVQDDMGMMRVMAAAPPAPQPETNTTIMFPSLKLSPTITLPSASLLSSLASPTPVAGGGPSILSSFQDDTPVGMASTPNLNRNPVKLGYYAFAGFGQLLNNNNDHTLINSQVIGASIQNATQSVTLPVDHPVTFTFQHLTTKGVSNPRCVYWDLMESKWSTLGCTLIATSSNSSQCSCTHLTSFAILMDISGQVGRLSGGLASALDVVSTIGCAISIVCLALSVCVFTFFRNLQNVRNSIHRNLCLCLLIAELVFVIGMDRTGNRTGCGVVAILLHYFFLSSFCWMLLEGYQLYMMLIQVFEPNRTRIFLYYLFCYGTPAVVVAISAGIKWEDYGTDSYCWIDTSTPTIWAFVAPIIVIIAANIIFLLIALKVVLSVQSRDRTKWGRIIGWLKGSATLLCLLGITWIFGFLTAVKGGTGTAFAWIFTILNCTQGIFIFVLHVVLNEKVRASIVRWLRTGICCLPETSSAAYNSRSFLSSRQRILNMIKVNGHSYPSTASTDDKEKQLTPITKTTDWLSRLPNQDSVSIPESNFNNLNGTLENSNLNSAEIKEEDEIPELRRRVTVDLNPMIVSNNEIERMSHASSDPRGSQIIEVTAVEKKAPVKRIKFPLGAKQSERGSQHRTKAKHGTGTLVSPWHIVTAAHLIGISEDPLPDCDTGNLREAYFVRDYKNFVAFVNVTCAVPEMCKGLHRKDMFKPLAIKSLYIRKGYVGDGCIDRESFNDIAVFELEEPIEFSKDIFPACLPSAPKIPRIRETGYKLFGYGRDPSDSVLESGKLKSLYSFVAECSDDFPYGGVYCTSAVNRGLSCDGDSGSGVVRTSDTRNVQVLVGVLSAGMPCPELYDTHNRQRQQRRQLTQETDLLVDVSAHVDFFCTCCGMCS

SEQ ID NO: 12 - protein, 198 aa, Homo sapiens
METYSLSLGNQSVVEPNIAIQSANFSSENAVGPSNVRFSVQKGASSSLVSSSTFIHTNVDGLNPDAQTELQVLLNMTKNYTKTCGFVVYQNDKLFQSKTFTAKSDFSQKIISSKTDENEQDQSASVDMVFSPKYNQKEFQLYSYACVYWNLSAKDWDTYGCQKDKGTDGFLRCRCNHTTNFAVLMTFKKDYQYPKSLD

SEQ ID NO: 13 - protein, 10 aa, Homo sapiens
QIVTRKVRKT

SEQ ID NO: 14 - protein, 38 aa, Homo sapiens
ENSNKNLQTSDGDINNIDFDNNDIPRTDTINIPNPMCT

SEQ ID NO: 15 - protein, 10 aa, Homo sapiens
IRTMKPLPRH

SEQ ID NO: 16 - protein, 41 aa, Homo sapiens
TVGVIYSQNGNNPQWELDYRQEKICWLAIPEPNGVIKSPLL

SEQ ID NO: 17 - protein, 25 aa, Homo sapiens
TISIKVLWKNNQNLTSTKKVSSMKK

SEQ ID NO: 18 - protein, 6 aa, Homo sapiens
NDDSIR

SEQ ID NO: 19 - protein, 78 aa, Homo sapiens
YTVRTKVFQSEASKVLMLLSSIGRRKSLPSVTRPRLRVKMYNFLRSLPTLHERFRLLETSPSTEEITLSESDNAKESI

SEQ ID NO: 20 - DNA, 38 bp, Homo sapiens
cgggatgcctagatgctttcctttgcattgtcactttc

SEQ ID NO: 21 - DNA, 66 bp, Homo sapiens
cggggatccctacttgtcgtcgtcgtccttgtagtccatgatgctttcctttgcattgtcactttc

SEQ ID NO: 22 - DNA, 23 bp, Homo sapiens
cagacaccattaacatcccgaat

SEQ ID NO: 23 - DNA, 22 bp, Homo sapiens
agaatgaaatgccgaggaagag

SEQ ID NO: 24 - DNA, 17 bp, Homo sapiens
agccgagccacatcgct

SEQ ID NO: 25 - DNA, 19 bp, Homo sapiens
gtgaccaggcgcccaatac

SEQ ID NO: 26 - DNA, 28 bp, Homo sapiens
caaatccgttgactccgaccttcacctt

SEQ ID NO: 27 - protein, 13 aa, Homo sapiens
QSKTFTAKSDFSQ

SEQ ID NO: 28 - protein, 13 aa, Homo sapiens
AKSDFSQKIISSK

SEQ ID NO: 29 - protein, 13 aa, Homo sapiens
SQKIISSKTDENE

SEQ ID NO: 30 - protein, 13 aa, Homo sapiens
VDMVFSPKYNQKE

SEQ ID NO: 31 - protein, 13 aa, Homo sapiens
VYWNLSAKDWDTY

SEQ ID NO: 32 - protein, 13 aa, Homo sapiens
FAVLMTFKKDYQY

SEQ ID NO: 33 - protein, 13 aa, Homo sapiens
IFQIVTRKVRKTS

SEQ ID NO: 34 - protein, 13 aa, Homo sapiens
FGIENSNKNLQTS

SEQ ID NO: 35 - protein, 13 aa, Homo sapiens
YLLIRTMKPLPRH

SEQ ID NO: 36 - protein, 13 aa, Homo sapiens
MFITISIKVLWKN

SEQ ID NO: 37 - protein, 13 aa, Homo sapiens
NQNLTSTKKVSSM

SEQ ID NO: 38 - protein, 13 aa, Homo sapiens
QNLTSTKKVSSMK

SEQ ID NO: 39 - protein, 13 aa, Homo sapiens
TKKVSSMKKIVST

SEQ ID NO: 40 - protein, 13 aa, Homo sapiens
LVNDDSIRIVFSY

SEQ ID NO: 41 - protein, 13 aa, Homo sapiens
IFILYTVRTKVFQ

SEQ ID NO: 42 - protein, 14 aa, Homo sapiens
SLGNQSVVEPNIAI

SEQ ID NO: 43 - protein, 14 aa, Homo sapiens
STFIHTNVDGLNPD

SEQ ID NO: 44 - protein, 14 aa, Homo sapiens
QKIISSKTDENEQD

SEQ ID NO: 45 - protein, 14 aa, Homo sapiens
VYWNLSAKDWDTYG

SEQ ID NO: 46 - protein, 14 aa, Homo sapiens
KNLQTSDGDINNID

SEQ ID NO: 47 - protein, 14 aa, Homo sapiens
LRSLPTLHERFRLL

SEQ ID NO: 48 - protein, 14 aa, Homo sapiens
LETSPSTEEITLSE

SEQ ID NO: 49 - protein, 14 aa, Homo sapiens
STEEITLSESDNAK

SEQ ID NO: 50 - protein, 14 aa, Homo sapiens
EEITLSESDNAKES

SEQ ID NO: 51 - protein, 14 aa, Homo sapiens
VTRKVRKTSVTWVL

SEQ ID NO: 52 - protein, 14 aa, Homo sapiens
NLTSTKKVSSMKKI

SEQ ID NO: 53 - protein, 14 aa, Homo sapiens
LSSIGRRKSLPSVT

SEQ ID NO: 54 - protein, 14 aa, Homo sapiens
SLSLGNQSVVEPNI

SEQ ID NO: 55 - protein, 14 aa, Homo sapiens
AIQSANFSSENAVG

SEQ ID NO: 56 - protein, 14 aa, Homo sapiens
LQVLLNMTKNYTKT

SEQ ID NO: 57 - protein, 14 aa, Homo sapiens
LNMTKNYTKTCGFV

SEQ ID NO: 58 - protein, 14 aa, Homo sapiens
ACVYWNLSAKDWDT

SEQ ID NO: 59 - protein, 14 aa, Homo sapiens
LRCRCNHTTNFAVL

SEQ ID NO: 60 - protein, 14 aa, Homo sapiens
WKNNQNLTSTKKVS

SEQ ID NO: 61 - protein, 14 aa, Homo sapiens
IFCLFNTTQGLQIF

SEQ ID NO: 62 - protein, 16 aa, Homo sapiens
FSVQKGASSSLVSSST

SEQ ID NO: 63 - protein, 16 aa, Homo sapiens
ILSNVGCALSVTGLAL

SEQ ID NO: 64 - protein, 16 aa, Homo sapiens
ALSVTGLALTVIFQIV

SEQ ID NO: 65 - protein, 16 aa, Homo sapiens
LLFVFGIENSNKNLQT

SEQ ID NO: 66 - protein, 16 aa, Homo sapiens
VAITVGVIYSQNGNNP

SEQ ID NO: 67 - DNA, 99 bp, Artificial Sequence
  Synthesized Oligonucleotide.
cgaagcgtaagggcccagccggccnnknnknnknnknnknnknnknnknnknnknnknnknnknnknnknnknnknnknnknnkccgggtccgggcggc

SEQ ID NO: 68 - DNA, 98 bp, Artificial Sequence
  Synthesized Oligonucleotide.
aaaaggaaaaaagcggccgcvnnvnnvnnvnnvnnvnnvnnvnnvnnvnnvnnvnnvnnvnnvnnvnnvnnvnnvnnvnnvnngccgcccggacccgg

SEQ ID NO: 69 - protein, 5 aa, Artificial Sequence
  Synthesized Peptide.
PGPGG

SEQ ID NO: 70 - protein, 15 aa, Artificial Sequence
  Synthesized Peptide.
FAGQIIWYDALDTLM

SEQ ID NO: 71 - protein, 15 aa, Artificial Sequence
  Synthesized Peptide.
SDFVGGFWFWDSLFN

SEQ ID NO: 72 - protein, 15 aa, Artificial Sequence
  Synthesized Peptide.
GDFWYEACESSCAFW

SEQ ID NO: 73 - protein, 15 aa, Artificial Sequence
  Synthesized Peptide.
LEWGSDVFYDVYDCC

SEQ ID NO: 74 - protein, 14 aa, Artificial Sequence
  Synthesized Peptide.
RIDSCAKYFLRSCD

SEQ ID NO: 75 - protein, 15 aa, Artificial Sequence
  Synthesized Peptide.
CLRSGTGCAFQLYRF

SEQ ID NO: 76 - protein, 15 aa, Artificial Sequence
  Synthesized Peptide.
FRVSRVWNPPSFDSA

SEQ ID NO: 77 - protein, 15 aa, Artificial Sequence
  Synthesized Peptide.
HAYVECNDTDCRVWF

SEQ ID NO: 78 - DNA, 39 bp, Homo sapiens
gcagcagcggccgcgacatattatccaacgttggatgtg

SEQ ID NO: 79 - DNA, 35 bp, Homo sapiens
gcagcagtcgacgatgctttcctttgcattgtcac

SEQ ID NO: 80 - DNA, 39 bp, Homo sapiens
gcagcagcggccgcatggagacttattccttgtctttgg

SEQ ID NO: 81 - DNA, 37 bp, Homo sapiens
gcagcagtcgacgtacaggataaaaatttgcaatccc

SEQ ID NO: 82 - DNA, 25 bp, Artificial Sequence
  Synthesized Oligonucleotide.
gguccaguuacaccaucaagaggcu

SEQ ID NO: 83 - DNA, 25 bp, Artificial Sequence
  Synthesized Oligonucleotide.
cauagauacugcccugagacccgca

SEQ ID NO: 84 - DNA, 25 bp, Artificial Sequence
  Synthesized Oligonucleotide.
ccuacccugcccaucugugauuagg

SEQ ID NO: 85 - DNA, 25 bp, Artificial Sequence
  Synthesized Oligonucleotide.
cccuaguguuagcucuuccaugcau

SEQ ID NO: 86 - DNA, 25 bp, Artificial Sequence
  Synthesized Oligonucleotide.
ccaggcacaugaaaggcuguacaua

SEQ ID NO: 87 - DNA, 21 bp, Homo sapiens
ctggagactctcagggtcgaa

SEQ ID NO: 88 - DNA, 17 bp, Homo sapiens
gcgcttccaggactgca

SEQ ID NO: 89 - DNA, 27 bp, Homo sapiens
acagatttctaccactccaaacgccgg

SEQ ID NO: 90 - protein, 15 aa, Artificial Sequence
  Synthesized Peptide.
YYSTPSYADVFRFVW

SEQ ID NO: 91 - protein, 15 aa, Artificial Sequence
  Synthesized Peptide.
YFDWTEVPYLMLEEA

(19) United States
(12) Patent Application Publication
(10) Pub. No.: US 2003/0170671 A1
(43) Pub. Date: Sep. 11, 2003

(54) Novel human G-protein coupled receptor, HGPRBMY6, expressed highly in small intestine

(76) Inventors: John N. Feder, Belle Mead, NJ (US); Gabriel Mintier, Hightstown, NJ (US); Chandra S. Ramanathan, Wallingford, CT (US); Donald R. Hawken, Lawrenceville, NJ (US); Angela M. Cacace, Clinton, CT (US); Lauren E. Barber, Jewett City, CT (US); Michael G. Kornacker, Princeton, NJ (US); Thomas C. Nelson, Lawrenceville, NJ (US); David K. Bol, Langhorne, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/262,272

(22) Filed: Sep. 27, 2002

Related U.S. Application Data
(63) Continuation-in-part of application No. 09/966,422, filed on Sep. 26, 2001.
(60) Provisional application No. 60/235,602, filed on Sep. 27, 2000.
(60) Provisional application No. 60/306,604, filed on Jul. 19, 2001.
(60) Provisional application No. 60/315,412, filed on Aug. 28, 2001.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/350; 536/23.5

(57) ABSTRACT

The present invention describes a newly discovered human G-protein coupled receptor and its encoding polynucleotide. Also described are expression vectors, host cells, agonists, antagonists, antisense molecules, and antibodies associated with the polynucleotide and/or polypeptide of the present invention. In addition, methods for treating, diagnosing, preventing and screening for disorders associated with aberrant cell growth and those related to the small intestine and colon are illustrated.

This application claims benefit to non-provisional application U.S. Ser. No. 09/966,422 filed Sep. 26, 2001, which claims benefit to provisional application U.S. Serial No. 60/235,602, filed Sep. 27, 2000; to provisional application U.S. Serial No. 60/306,604, filed Jul. 19, 2001; and to provisional application U.S. Serial No. 60/315,412, filed Aug. 28, 2001. The entire teachings of the referenced applications are incorporated herein by reference.

### FIELD OF THE INVENTION

The present invention relates to the fields of pharmacogenomics, diagnostics and patient therapy. More specifically, the present invention relates to methods of diagnosing and/or treating diseases involving the Human G-Protein Coupled Receptor, HGPRBMY6.

### BACKGROUND OF THE INVENTION

It is well established that many medically significant biological processes are mediated by proteins participating in signal transduction pathways that involve G-proteins and/or second messengers, e.g., cAMP (Lefkowitz, Nature, 351:353-354 (1991)). Herein these proteins are referred to as proteins participating in pathways with G-proteins or PPG proteins. Some examples of these proteins include the GPC receptors, such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding is activation of an enzyme, adenylate cyclase, inside the cell. Enzyme activation by hormones is dependent on the presence of the nucleotide GTP, and GTP also influences hormone binding. A G-protein connects the hormone receptors to adenylate cyclase. G-protein was shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role, as an intermediate that relays the signal from receptor to effector, and as a clock that controls the duration of the signal.

The membrane protein gene superfamily of G-protein coupled receptors has been characterized as having seven putative transmembrane domains. The domains are believed to represent transmembrane a-helices connected by extracellular or cytoplasmic loops. G-protein coupled receptors include a wide range of biologically active receptors, such as hormone, viral, growth factor and neuroreceptors.

G-protein coupled receptors have been characterized as including these seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family include calcitonin, adrenergic, endothelin, cAMP, adenosine, muscarinic, acetylcholine, serotonin, histamine, thrombin, kinin, follicle stimulating hormone, opsins, endothelial differentiation gene-i receptor, rhodopsins, odorant, cytomegalovirus receptors, etc.

Most G-protein coupled receptors have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or farnesylation) of cysteine residues can influence signal transduction of some G-protein coupled receptors. Most G-protein coupled receptors contain potential phosphorylation sites within the third cytoplasmic loop and/or the carboxyl terminus. For several G-protein coupled receptors, such as the β-adrenoreceptor, phosphorylation by protein kinase A and/or specific receptor kinases mediates receptor desensitization.

For some receptors, the ligand binding sites of G-protein coupled receptors are believed to comprise a hydrophilic socket formed by several G-protein coupled receptors transmembrane domains, which socket is surrounded by hydrophobic residues of the G-protein coupled receptors. The hydrophilic side of each G-protein coupled receptor transmembrane helix is postulated to face inward and form the polar ligand-binding site. TM3 has been implicated in several G-protein coupled receptors as having a ligand-binding site, such as including the TM3 aspartate residue. Additionally, TM5 serines, a TM6 asparagine and TM6 or TM7 phenylalanines or tyrosines are also implicated in ligand binding.

G-protein coupled receptors can be intracellularly coupled by heterotrimeric G-proteins to various intracellular enzymes, ion channels and transporters (see, Johnson et al., Endoc. Rev., 10:317-331(1989)). Different G-protein β-subunits preferentially stimulate particular effectors to modulate various biological functions in a cell. Phosphorylation of cytoplasmic residues of G-protein coupled receptors have been identified as an important mechanism for the regulation of G-protein coupling of some G-protein coupled receptors. G-protein coupled receptors are found in numerous sites within a mammalian host.

G-protein coupled receptors (GPCRs) are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)). The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

Characterization of the HGPRBMY6 polypeptide of the present invention led to the determination that it is involved in the modulation of the p21 G1/S-phase cell cycle check point protein, either directly or indirectly.

Critical transitions through the cell cycle are highly regulated by distinct protein kinase complexes, each composed of a cyclin regulatory and a cyclin-dependent kinase (cdk) catalytic subunit (for review see Draetta, Curr. Opin. Cell Biol. 6, 842-846 (1994)). These proteins regulate the cell's progression through the stages of the cell cycle and are, in turn, regulated by numerous proteins, including p53, p21, p16, and cdc25. Downstream targets of cyclin-cdk complexes include pRb and E2F. The cell cycle often is dysregulated in neoplasia due to alterations either in oncogenes that indirectly affect the cell cycle, or in tumor suppressor genes or oncogenes that directly impact cell cycle regulation, such as pRb, p53, p16, cyclin D1, or mdm-2 (for review see Schafer, Vet Pathol 1998 35, 461-478 (1998)).

P21, also known as CDNK1A (cyclin-dependent kinase inhibitor 1A), or CIP1 inhibits mainly the activity of cyclin CDK2 or CDK4 complexes. Therefore, p21 primarily blocks cell cycle progression at the G1 stage of the cell cycle. The expression of p21 is tightly controlled by the tumor suppressor protein p53, through which this protein mediates the cell cycle G1 phase arrest in response to a variety of stress stimuli. In addition, p21 protein interacts with the DNA polymerase accessory factor PCNA (proliferating cell nuclear antigen), and plays a regulatory role in S phase DNA replication and DNA damage repair.

After DNA damage, many cells appear to enter a sustained arrest in the G2 phase of the cell cycle. Bunz et al. (Science 282, 1497-1501 (1998)) demonstrated that this arrest could be sustained only when p53 was present in the cell and capable of transcriptionally activating the cyclin-dependent kinase inhibitor p21. After disruption of either the p53 or the p21 gene, gamma-radiated cells progressed into mitosis and exhibited a G2 DNA content only because of a failure of cytokinesis. Thus, p53 and p21 appear to be essential for maintaining the G2 cell cycle checkpoint in human cells.

Due to the connection between the transcriptional activity of p53 and p21 RNA expression, the readout of p21, RNA can be used to determine the effect of drugs or other insults (radiation, antisense for a specific gene, dominant negative expression) on a given cell system which contains wild type p53. Specifically, if a gene is removed using antisense products and this has an effect on the p53 activity, p21 will be upregulated and can serve therefore as an indirect marker for an influence on the cell cycle regulatory pathways and induction of cell cycle arrest.

In addition to cancer regulation of cell cycle activity has a role in numerous other systems. For example, hematopoietic stem cells are relative quiescent, while after receiving the required stimulus they undergo dramatic proliferation and inexorably move toward terminal differentiation. This is partly regulated by the presence of p21. Using p21 knockout mice Cheng et al. (Science 287, 1804-1808 (2000)) demonstrated its critical biologic importance in protecting the stem cell compartment. In the absence of p21, hematopoietic stem cell proliferation and absolute number were increased under normal homeostatic conditions. Exposing the animals to cell cycle-specific myelotoxic injury resulted in premature death due to hematopoietic cell depletion. Further, self-renewal of primitive cells was impaired in serially transplanted bone marrow from p21 −/− mice, leading to hematopoietic failure. Therefore it was concluded that p21 is the molecular switch governing the entry of stem cells into the cell cycle, and in its absence, increased cell cycling leads to stem cell exhaustion. Under conditions of stress, restricted cell cycling is crucial to prevent premature stem cell depletion and hematopoietic death. Therefore, genes involved in the downregulation of p21 expression could have a stimulatory effect and therefore be useful for the exploration of stem cell technologies.

The present invention provides a newly-discovered G-protein coupled receptor protein, which may be involved in cellular growth properties in the small intestine, as well as in other gastrointestinal tissues, such as colon, based on its abundance in the small intestine, colon, and in addition to colon cancer cell lines. This invention also relates to newly identified polynucleotides, polypeptides encoded by such polynucleotides, the use of such polynucleotides and polypeptides, as well as the production of such polynucleotides and polypeptides. More particularly, the polypeptides of the present invention are human 7-transmembrane receptors. In addition, the invention also relates to inhibiting the action of such polypeptides.

### SUMMARY OF THE INVENTION

The present invention describes a novel human member of the G-protein coupled receptor (GPCR) family (HGPRBMY6). Based on sequence homology, the protein HGPRBMY6 is a candidate GPCR. The HGPRBMY6 sequence has been predicted to contain seven transmembrane domains which is a characteristic structural feature of GPCRs. HGPRBMY6 is related to latrophilin, alpha-latrotoxin, and CL3 receptors based on sequence similarity. This orphan GPCR is expressed highly in small intestine and colonic tissues.

The present invention provides an isolated HGPRBMY6 polynucleotide as depicted in SEQ ID NO:1.

The present invention also provides the HGPRBMY6 polypeptide (MW: 63.2 Kd), encoded by the polynucleotide of SEQ ID NO:1 and having the amino acid sequence of SEQ ID NO:2, or a functional or biologically active portion thereof.

The present invention further provides compositions comprising the HGPRBMY6 polynucleotide sequence, or a fragment thereof, or the encoded HGPRBMY6 polypeptide, or a fragment or portion thereof. Also provided by the present invention are pharmaceutical compositions comprising at least one HGPRBMY6 polypeptide, or a functional portion thereof, wherein the compositions further comprise a pharmaceutically acceptable carrier, excipient, or diluent.

The present invention provides a novel isolated and substantially purified polynucleotide that encodes the HGPRBMY6 GPCR homologue. In a particular aspect, the polynucleotide comprises the nucleotide sequence of SEQ ID NO: 1. The present invention also provides a polynucleotide sequence comprising the complement of SEQ ID NO: 1, or variants thereof. In addition, the present invention features polynucleotide sequences, which hybridize under moderately stringent or high stringency conditions to the polynucleotide sequence of SEQ ID NO:1.

The present invention further provides a nucleic acid sequence encoding the HGPRBMY6 polypeptide and an antisense of the nucleic acid sequence, as well as oligonucleotides, fragments, or portions of the nucleic acid molecule or antisense molecule. Also provided are expression vectors and host cells comprising polynucleotides that encode the HGPRBMY6 polypeptide.

The present invention provides methods for producing a polypeptide comprising the amino acid sequence depicted in SEQ ID NO:2, or a fragment thereof, comprising the steps of a) cultivating a host cell containing an expression vector containing at least a functional fragment of the polynucleotide sequence encoding the HGPRBMY6 protein according to this invention under conditions suitable for the expression of the polynucleotide; and b) recovering the polypeptide from the host cell.

Also provided are antibodies, and binding fragments thereof, which bind specifically to the HGPRBMY6 polypeptide, or an epitope thereof, for use as therapeutics and diagnostic agents.

The present invention also provides methods for screening for agents which modulate HGPRBMY6 polypeptide, e.g., agonists and antagonists, as well as modulators, e.g., agonists and antagonists, particularly those that are obtained from the screening methods described.

Also provided by the present invention is a substantially purified antagonist or inhibitor of the polypeptide of SEQ ID NO:2. In this regard, and by way of example, a purified antibody that binds to a polypeptide comprising the amino acid sequence of SEQ ID NO:2 is provided.

Substantially purified agonists of the G-protein coupled receptor polypeptide of SEQ ID NO:2 are further provided.

The present invention provides HGPRBMY6 nucleic acid sequences, polypeptide, peptides and antibodies for use in the diagnosis and/or screening of disorders or diseases associated with expression of the polynucleotide and its encoded polypeptide as described herein.

The present invention provides kits for screening and diagnosis of disorders associated with aberrant or uncontrolled cellular development and with the expression of the polynucleotide and its encoded polypeptide as described herein.

The present invention further provides methods for the treatment or prevention of cancers, immune disorders, neurological, small intestine-related, or colon-related disorders, pulmonary disorders, lung cancers, reproductive disorders, ovarian cancers, diseases, or conditions involving administering, to an individual in need of treatment or prevention, an effective amount of a purified antagonist of the HGPRBMY6 polypeptide. Due to its elevated expression in small intestine and colon, the novel GPCR protein of the present invention is particularly useful in treating or preventing gastrointestinal disorders, conditions, or diseases, including Crohn's disease, inflammatory bowel disease, sacroiliitis, ileitis, malabsorption disorders, short bowel syndrome, celiac sprue, and small intestinal motility disorders. Moreover, the elevated expression in colon cancer cell lines, combined with the association of HGPRBMY6 to the modulation of P21, suggests modulators, agonists or antagonists, that increase or alternatively decrease expression of HGPRBMY6, or modulators that increase or alternatively decrease activity of HGPRBMY6 are useful for the treatment of proliferative disorders of the gastrointestinal system, particularly of the colon and small intestine such as colon cancer, etc.

The present invention also provides a method for detecting a polynucleotide that encodes the HGPRBMY6 polypeptide in a biological sample comprising the steps of: a) hybridizing the complement of the polynucleotide sequence encoding SEQ ID NO:2 to a nucleic acid material of a biological sample, thereby forming a hybridization complex; and b) detecting the hybridization complex, wherein the presence of the complex correlates with the presence of a polynucleotide encoding the HGPRBMY6 polypeptide in the biological sample. The nucleic acid material may be further amplified by the polymerase chain reaction prior to hybridization.

Further objects, features, and advantages of the present invention will be better understood upon a reading of the detailed description of the invention when considered in connection with the accompanying figures/drawings.

One aspect of the instant invention comprises methods and compositions to detect and diagnose alterations in the HGPRBMY6 sequence in tissues and cells as they relate to ligand response.

The present invention further provides compositions for diagnosing small intestine- and colon-related disorders and response to HGPRBMY6 therapy in humans. In accordance with the invention, the compositions detect an alteration of the normal or wild type HGPRBMY6 sequence or its expression product in a patient sample of cells or tissue.

The present invention further provides compositions for diagnosing pulmonary and lung-related disorders and response to HGPRBMY6 therapy in humans. In accordance with the invention, the compositions detect an alteration of the normal or wild type HGPRBMY6 sequence or its expression product in a patient sample of cells or tissue.

The present invention further provides compositions for diagnosing reproductive and ovarian-related disorders and response to HGPRBMY6 therapy in humans. In accordance with the invention, the compositions detect an alteration of the normal or wild type HGPRBMY6 sequence or its expression product in a patient sample of cells or tissue.

The present invention further provides diagnostic probes for diseases and a patient's response to therapy. The probe sequence comprises the HGPRBMY6 locus polymorphism. The probes can be constructed of nucleic acids or amino acids.

The present invention further provides antibodies that recognize and bind to the HGPRBMY6 protein. Such antibodies can be either polyclonal or monoclonal. Antibodies that bind to the HGPRBMY6 protein can be utilized in a variety of diagnostic and prognostic formats and therapeutic methods.

The present invention is directed to peptides that bind to the HGPRBMY6 polypeptide of the present invention having a polypeptide sequence selected from the group consisting of SEQ ID NO:70-77, 90, and/or 91, wherein said peptides modulate the activity of the HGPRBMY6 polypeptide.

The present invention is directed to peptides that bind to the HGPRBMY6 polypeptide of the present invention having a polypeptide sequence selected from the group consisting of SEQ ID NO:70-77, 90, and/or 91, wherein said peptides agonize the activity of the HGPRBMY6 polypeptide.

The present invention is directed to peptides that bind to the HGPRBMY6 polypeptide of the present invention having a polypeptide sequence selected from the group consisting of SEQ ID NO:70-77, 90, and/or 91, wherein said peptides antagonize the activity of the HGPRBMY6 polypeptide.

The present invention is directed to peptides that bind to the HGPRBMY6 polypeptide of the present invention having a polypeptide sequence selected from the group consisting of SEQ ID NO:70-77, 90, and/or 91, wherein said peptides are useful as competitive inhibitors of one or more ligands of the HGPRBMY6 polypeptide.

The present invention is directed to peptides that bind to the HGPRBMY6 polypeptide of the present invention having a polypeptide sequence selected from the group consisting of SEQ ID NO:70-77, 90, and/or 91, wherein said peptides are useful as non-competitive inhibitors of one or more ligands of the HGPRBMY6 polypeptide.

The present invention is directed to peptides that bind to the HGPRBMY6 polypeptide of the present invention having a polypeptide sequence selected from the group consisting of SEQ ID NO:70-77, 90, and/or 91, wherein said peptides are capable of serving as a surrogate ligand of the HGPRBMY6 polypeptide.

The present invention is directed to a method of using a peptide that binds to the HGPRBMY6 polypeptide of the present invention, to treat or ameliorate a disease or disorder, having a polypeptide sequence selected from the group consisting of SEQ ID NO:70-77, 90, and/or 91, wherein said peptides antagonize the activity of the HGPRBMY6 polypeptide.

The present invention is directed to a method of using a peptide that binds to the HGPRBMY6 polypeptide of the present invention, to treat or ameliorate a disease or disorder, having a polypeptide sequence selected from the group consisting of SEQ ID NO:70-77, 90, and/or 91, wherein said peptides agonize the activity of the HGPRBMY6 polypeptide.

The present invention is also directed to a method of identifying a compound that modulates the biological activity of HGPRBMY6, comprising the steps of, (a) combining a candidate modulator compound with HGPRBMY6 in the presence of a peptide that antagonizes the activity of the HGPRBMY6 polypeptide selected from the group consisting of SEQ ID NO:70-77, 90, and/or 91, and (b) identifying candidate compounds that reverse the antagonizing effect of the peptide.

The invention further relates to a method of identifying a compound that modulates the biological activity of HGPRBMY6, comprising the steps of, (a) combining a candidate modulator compound with HGPRBMY6 in the presence of a peptide that agonizes the activity of the HGPRBMY6 polypeptide selected from the group consisting of SEQ ID NO:70-77, 90, and/or 91, and (b) identifying candidate compounds that reverse the agonizing effect of the peptide.

The present invention is also directed to a method of identifying a compound that modulates the biological activity of HGPRBMY6, comprising the steps of, (a) combining a candidate modulator compound with HGPRBMY6 in the presence of an antisense molecule that antagonizes the activity of the HGPRBMY6 polypeptide selected from the group consisting of SEQ ID NO:70-77, 90, and/or 91, and (b) identifying candidate compounds that reverse the antagonizing effect of the peptide.

The present invention is also directed to a method of identifying a compound that modulates the biological activity of HGPRBMY6, comprising the steps of, (a) combining a candidate modulator compound with HGPRBMY6 in the presence of a small molecule that antagonizes the activity of the HGPRBMY6 polypeptide selected from the group consisting of SEQ ID NO:70-77, 90, and/or 91, and (b) identifying candidate compounds that reverse the antagonizing effect of the peptide.

The present invention is also directed to a method of identifying a compound that modulates the biological activity of HGPRBMY6, comprising the steps of, (a) combining a candidate modulator compound with HGPRBMY6 in the presence of a small molecule that agonizes the activity of the HGPRBMY6 polypeptide selected from the group consisting of SEQ ID NO:70-77, 90, and/or 91, and (b) identifying candidate compounds that reverse the agonizing effect of the peptide.

The present invention also provides diagnostic kits for the determination of the nucleotide sequence of human HGPRBMY6 alleles. The kits are based on amplification-based assays, nucleic acid probe assays, protein nucleic acid probe assays, antibody assays or any combination thereof.

The instant invention also provides methods for detecting genetic predisposition, susceptibility and response to therapy related to the small intestine colon, lung or ovary. In accordance with the invention, the method comprises isolating a human sample, for example, blood or tissue from adults, children, embryos or fetuses, and detecting at least one alteration in the wild-type HGPRBMY6 sequence or its expression product from the sample, wherein the alterations are indicative of genetic predisposition, susceptibility or altered response to therapy related to the small intestine and colon.

In addition, methods for making determinations as to which drug to administer, dosages, duration of treatment and the like are provided.

The present invention further relates to a method for preventing, treating, or ameliorating a medical condition with the polypeptide provided as SEQ ID NO:2, in addition to, its encoding nucleic acid, or a modulator thereof, wherein the medical condition is a disorder associated with p21 expression or activity.

The invention further relates to a method for preventing, treating, or ameliorating a medical condition with the polypeptide provided as SEQ ID NO:2, in addition to, its encoding nucleic acid, or a modulator thereof, wherein the medical condition is a cell cycle defect, disorders related to phosphorylation, disorders related to signal transduction, proliferating disorders, and/or cancers.

The invention further relates to a method for preventing, treating, or ameliorating a medical condition with the polypeptide provided as SEQ ID NO:2, in addition to, its encoding nucleic acid, or a modulator thereof, wherein the medical condition is a disorder associated with cell cycle regulation.

The invention further relates to a method of increasing, or alternatively decreasing, the number of cells in the G1 phase of the cell cycle comprising the step of administering an antagonist, or alternatively an agonist, of HGPRBMY6.

The invention further relates to a method of increasing, or alternatively decreasing, the number of cells in the G2 phase of the cell cycle comprising the step of administering an antagonist, or alternatively an agonist, of HGPRBMY6.

The invention further relates to a method of decreasing, or alternatively increasing, the number of cells that progress to the S phase of the cell cycle comprising the step of administering an antagonist, or, alternatively an agonist, of HGPRBMY6.

The invention further relates to a method of increasing, or alternatively decreasing, the number of cells that progress to the M phase of the cell cycle comprising the step of administering an antagonist, or alternatively an agonist, of HGPRBMY6.

The invention further relates to a method of inducing, or alternatively inhibiting, cells into G1 and/or G2 phase arrest comprising the step of administering an antagonist, or alternatively an agonist, of HGPRBMY6.

The invention also relates to an antisense compound 8 to 30 nucleotides in length that specifically hybridizes to a nucleic acid molecule encoding the human HGPRBMY6 polypeptide of the present invention, wherein said antisense compound inhibits the expression of the human HGPRBMY6 polypeptide.

The invention further relates to a method of inhibiting the expression of the human HGPRBMY6 polypeptide of the present invention in human cells or tissues comprising contacting said cells or tissues in vitro or in vivo, with an antisense compound of the present invention so that expression of the HGPRBMY6 polypeptide is inhibited.

The invention further relates to a method of increasing the expression of p21 in human cells or tissues comprising contacting said cells or tissues in vitro, or in vivo, with an antisense compound that specifically hybridizes to a nucleic acid molecule encoding the human HGPRBMY6 polypeptide of the present invention so that expression of the HGPRBMY6 polypeptide is inhibited.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are HEK cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, and futher wherein said cells express the polypeptide at either low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said cells express beta lactamase at low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2, or encoded by ATCC deposit PTA-2677, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said cells express beta lactamase at low, moderate, or high levels.

The file of this patent contains at least one Figure executed in color. Copies of this patent with color Figure(s) will be provided by the Patent and Trademark Office upon request and payment of the necessary fee.

### BRIEF DESCRIPTION OF THE FIGURES

FIG. 1 shows the full length nucleotide sequence of cDNA clone HGPRBMY6, a human G-protein coupled receptor (SEQ ID NO: 1).

FIG. 2 shows the amino acid sequence (SEQ ID NO:2) from the conceptual translation of the full length HGPRBMY6 cDNA sequence.

FIG. 3 shows the 5′ untranslated sequence of the orphan receptor, HGPRBMY6 (SEQ ID NO:3).

FIG. 4 shows the 3′ untranslated sequence of the orphan receptor, HGPRBMY6 (SEQ ID NO:4).

FIG. 5 shows the predicted transmembrane region of the HGPRBMY6 protein where the predicted transmembranes, bold-faced and underlined, correspond to the peaks with scores above 1500.

FIGS. 6A-6D show the multiple sequence alignment of the translated sequence of the orphan G-protein coupled receptor, HGPRBMY6, where the GCG pileup program was used to generate the alignment with other G-protein coupled receptor sequences. The blackened areas represent identical amino acids in more than half of the listed sequences and the grey highlighted areas represent similar amino acids. As shown in FIGS. 6A-6D, the sequences are aligned according to their amino acids, where: HGPRBMY6 (SEQ ID NO:2) is the translated full length HGPRBMY6 cDNA; SWISS Prot. Accesion No. O88925 (SEQ ID NO:8) represents the rat form of CL3AB; SWISS Prot. Accesion No. O88927 (SEQ ID NO:9) is the rat form of latrophilin 3; SWISS Prot. Accesion No. Q9Y3KO (SEQ ID NO:10) is the human form of SWISS Prot. Accesion No. DJ287G14.2, a novel seven transmembrane protein; and SWISS Prot. Accesion No. Q10922 (SEQ ID NO:11) is a protein, with weak similarity to GPCRs, from C. elegans.

FIG. 7 shows the expression profile of the novel human orphan GPCR, HGPRBMY6, amongst normal tissues, as described in Example 3.

FIG. 8 shows the expression profile of the novel human orphan GPCR, HGPRBMY6, amongst a variety of tumor cell lines, as described in Table I and Example 4.

FIG. 9 shows the FACS profile for an untrasfected CHO-NFAT/CRE cell line.

FIG. 10 shows the overexpression of HGPRBMY6 constitutively couples through the NFAT/CRE response element.

FIG. 11 shows the FACS profile for an untransfected cAMP Response Element.

FIG. 12 shows the FACS profile describing that HGPRBMY6 couples through the cAMP Response Element.

FIG. 13 shows the FACS profile for an untransfected CHO-NFAT G alpha 15 cell line.

FIG. 14 shows the overexpression of HGPRBMY6 constitutively coupled NFAT Response Element via the promiscuous G protein, G alpha 15.

FIG. 15 shows expressed HGPRBMY26 polypeptide localizes to the cell membrane. Cho-NFAT G alpha 15 cell lines transfected with the pcDNA3.1 Hygro™/HGPRBMY26-FLAG mammalian expression vector were subjected to immunocytochemistry using an FITC conjugated Anti Flag monoclonal antibody, as described herein. Panel A shows the transfected Cho-NFAT/CRE cells under visual wavelengths, and panel B shows the fluorescent emission of the same cells at 530 nm after illumination with a mercury light source. The cellular localization is clearly evident in panel B, and is consistent with the expression of HGPRBMY26.

FIG. 16 shows representative transfected Cho-NFAT/CRE cell lines with intermediate and high beta lactamase expression levels useful in screens to identify HGPRBMY26 agonists and/or antagonists. Several Cho-NFAT/CRE cell lines transfected with the pcDNA3.1 Hygro™/HGPRBMY26 mammalian expression vector were isolated via FACS that had either intermediate or high beta lactamase expression levels of constitutive activation, as described herein. Panel A shows untransfected Cho-NFAT/CRE cells prior to stimulation with 10 nM PMA and 1 uM Thapsigargin/10 uM Forskolin (−P/T/F). Panel B shows Cho-NFAT/CRE cells after stimulation with 10 nM PMA and 1 uM Thapsigargin/10 uM Forskolin (+P/T/F). Panel C shows a representative orphan GPCR (oGPCR) transfected Cho-NFAT/CRE cells that have an intermediate level of beta lactamase expression. Panel D shows a representative orphan GPCR transfected Cho-NFAT/CRE that have a high level of beta lactamase expression. Such cell lines with intermediate and high beta lactamase expression levels are useful in screens to identify HGPRBMY6 agonists and/or antagonists.

FIG. 17 depicts the graphical analysis of expression profile of the novel orphan GPCR, HGPRBMY6, amongst an expanded panel of tumor cell lines. The numbers on the y axis correspond to the numerical order of the cell lines in Table 2

FIG. 18 presents an expanded expression profile of the novel orphan GPCR, HGPRBMY6 in normal tissue.

### DETAILED DESCRIPTION OF THE INVENTION

The present invention provides a novel isolated polynucleotide and encoded polypeptide, the expression of which is high in small intestine, colonic tissues, and particularly in colon cancer. This novel polypeptide is termed herein HGPRBMY6, an acronym for “Human G-Protein coupled Receptor BMY6”. HGPRBMY6 is also referred to as GPCR29.

### Definitions

The HGPRBMY6 polypeptide (or protein) refers to the amino acid sequence of substantially purified HGPRBMY6, which may be obtained from any species, preferably mammalian, and more preferably, human, and from a variety of sources, including natural, synthetic, semi-synthetic, or recombinant. Functional fragments of the HGPRBMY6 polypeptide are also embraced by the present invention.

The term “modulates” refers to the process whereby, there is an increase or alternatively a decrease in expression or an increase or alternatively a decrease in activity. A “modulator” induces this process.

An “agonist” refers to a molecule which, when bound to the HGPRBMY6 polypeptide, or a functional fragment thereof, increases or prolongs the duration of the effect of the HGPRBMY6 polypeptide. Agonists may include proteins, nucleic acids, carbohydrates, or any other molecules that bind to and modulate the effect of HGPRBMY6 polypeptide. An antagonist refers to a molecule which, when bound to the HGPRBMY6 polypeptide, or a functional fragment thereof, decreases the amount or duration of the biological or immunological activity of HGPRBMY6 polypeptide. “Antagonists” may include proteins, nucleic acids, carbohydrates, antibodies, or any other molecules that decrease or reduce the effect of HGPRBMY6 polypeptide.

“Nucleic acid sequence”, as used herein, refers to an oligonucleotide, nucleotide, or polynucleotide, and fragments or portions thereof, and to DNA or RNA of genomic or synthetic origin which may be single- or double-stranded, and represent the sense or anti-sense strand. By way of non-limiting example, fragments include nucleic acid sequences that are greater than 20-60 nucleotides in length, and preferably include fragments that are at least 70-100 nucleotides, or which are at least 1000 nucleotides or greater in length.

Similarly, “amino acid sequence” as used herein refers to an oligopeptide, peptide, polypeptide, or protein sequence, and fragments or portions thereof, and to naturally occurring or synthetic molecules. Amino acid sequence fragments are typically from about 5 to about 30, preferably from about 5 to about 15 amino acids in length and retain the biological activity or function of the HGPRBMY6 polypeptide.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule. In addition, the terms HGPRBMY6 polypeptide and HGPRBMY6 protein are used interchangeably herein to refer to the encoded product of the HGPRBMY6 nucleic acid sequence of the present invention.

A “variant” of the HGPRBMY6 polypeptide refers to an amino acid sequence that is altered by one or more amino acids. The variant may have “conservative” changes, wherein a substituted amino acid has similar structural or chemical properties, e.g., replacement of leucine with isoleucine. More rarely, a variant may have “non-conservative” changes, e.g., replacement of a glycine with a tryptophan. Minor variations may also include amino acid deletions or insertions, or both. Guidance in determining which amino acid residues may be substituted, inserted, or deleted without abolishing functional biological or immunological activity may be found using computer programs well known in the art, for example, DNASTAR software.

An “allele” or “allelic sequence” is an alternative form of the HGPRBMY6 nucleic acid sequence. Alleles may result from at least one mutation in the nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Altered” nucleic acid sequences encoding HGPRBMY6 polypeptide include nucleic acid sequences containing deletions, insertions and/or substitutions of different nucleotides resulting in a polynucleotide that encodes the same or a functionally equivalent HGPRBMY6 polypeptide. Altered nucleic acid sequences may further include polymorphisms of the polynucleotide encoding the HGPRBMY6 polypeptide; such polymorphisms may or may not be readily detectable using a particular oligonucleotide probe. The encoded protein may also contain deletions, insertions, or substitutions of amino acid residues, which produce a silent change and result in a functionally equivalent HGPRBMY6 protein. Deliberate amino acid substitutions may be made on the basis of similarity in polarity, charge, solubility, hydrophobicity, hydrophilicity, and/or the amphipathic nature of the residues, as long as the biological activity of HGPRBMY6 protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers” refer to a nucleic acid sequence, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

“Amplification” refers to the production of additional copies of a nucleic acid sequence and is generally carried out using polymerase chain reaction (PCR) technologies, which are well known and practiced in the art (see, D. W. Dieffenbach and G. S. Dveksler, 1995, PCR Primer, a Laboratory Manual, Cold Spring Harbor Press, Plainview, N.Y.).

“Microarray” is an array of distinct polynucleotides or oligonucleotides synthesized on a substrate, such as paper, nylon, or other type of membrane; filter; chip; glass slide; or any other type of suitable solid support.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNA and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

The term “consensus” refers to the sequence that reflects the most common choice of base or amino acid at each position among a series of related DNA, RNA or protein sequences. Areas of particularly good agreement often represent conserved functional domains.

A “deletion” refers to a change in either nucleotide or amino acid sequence and results in the absence of one or more nucleotides or amino acid residues. By contrast, an “insertion” (also termed “addition”) refers to a change in a nucleotide or amino acid sequence that results in the addition of one or more nucleotides or amino acid residues, as compared with the naturally occurring molecule. A “substitution” refers to the replacement of one or more nucleotides or amino acids by different nucleotides or amino acids.

A “derivative nucleic acid molecule” refers to the chemical modification of a nucleic acid encoding, or complementary to, the encoded HGPRBMY6 polypeptide. Such modifications include, for example, replacement of hydrogen by an alkyl, acyl, or amino group. A nucleic acid derivative encodes a polypeptide, which retains the essential biological and/or functional characteristics of the natural molecule. A derivative polypeptide is one, which is modified by glycosylation, pegylation, or any similar process that retains the biological and/or functional or immunological activity of the polypeptide from which it is derived.

The term “biologically active”, i.e., functional, refers to a protein or polypeptide or fragment thereof having structural, regulatory, or biochemical functions of a naturally occurring molecule. Likewise, “immunologically active” refers to the capability of the natural, recombinant, or synthetic HGPRBMY6, or any oligopeptide thereof, to induce a specific immune response in appropriate animals or cells, for example, to generate antibodies, and to bind with specific antibodies.

The term “hybridization” refers to any process by which a strand of nucleic acid binds with a complementary strand through base pairing.

The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (e.g., Cₒt or Rₒt analysis), or between one nucleic acid sequence present in solution and another nucleic acid sequence immobilized on a solid support (e.g., membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (e.g., formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency, that are different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511). As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, e.g., high, moderate, or low stringency, typically relates to such washing conditions.

Thus, by way of non-limiting example, “high stringency” refers to conditions that permit hybridization of those nucleic acid sequences that form stable hybrids in 0.018M NaCl at about 65° C. (i.e., if a hybrid is not stable in 0.018M NaCl at about 65° C., it will not be stable under high stringency conditions). High stringency conditions can be provided, for instance, by hybridization in 50% formamide, 5× Denhardt's solution, 5×SSPE (saline sodium phosphate EDTA) (1×SSPE buffer comprises 0.15 M NaCl, 10 mM Na₂HPO₄, 1 mM EDTA), (or 1×SSC buffer containing 150 mM NaCl, 15 mM Na₃ citrate.2H₂O, pH 7.0), 0.2% SDS at about 42° C., followed by washing in 1×SSPE (or saline sodium citrate, SSC) and 0.1% SDS at a temperature of at least about 42° C., preferably about 55° C., more preferably about 65° C.

“Moderate stringency” refers, by non-limiting example, to conditions that permit hybridization in 50% formamide, 5× Denhardt's solution, 5×SSPE (or SSC), 0.2% SDS at 42° C. (to about 50° C.), followed by washing in 0.2×SSPE (or SSC) and 0.2% SDS at a temperature of at least about 42° C., preferably about 55° C., more preferably about 65° C.

Low stringency refers, by non-limiting example, to conditions that permit hybridization in 10% formamide, 5× Denhardt's solution, 6×SSPE (or SSC), 0.2% SDS at 42° C., followed by washing in 1×SSPE (or SSC) and 0.2% SDS at a temperature of about 45° C., preferably about 50° C.

For additional stringency conditions, see T. Maniatis et al., Molecular Cloning: A Laboratory Manual, Cold Spring Harbor Laboratory, Cold Spring Harbor, N.Y. (1982). It is to be understood that the low, moderate and high stringency hybridization/washing conditions may be varied using a variety of ingredients, buffers and temperatures well known to and practiced by the skilled artisan.

The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to using the functional term “substantially homologous”. The inhibition of hybridization of the completely complementary sequence to the target sequence may be examined using a hybridization assay (e.g., Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (e.g., less than about 30% identity). In the absence of non-specific binding, the probe will not hybridize to the second non-complementary target sequence.

Those having skill in the art will know how to determine percent identity between or among sequences using, for example, algorithms such as those based on the CLUSTALW computer program (J. D. Thompson et al., 1994, Nucleic Acids Research, 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations.

A “composition comprising a given polynucleotide sequence” refers broadly to any composition containing the given polynucleotide sequence. The composition may comprise a dry formulation or an aqueous solution. Compositions comprising polynucleotide sequence (SEQ ID NO:1) encoding HGPRBMY6 polypeptide (SEQ ID NO:2), or fragments thereof, may be employed as hybridization probes. The probes may be stored in freeze-dried form and may be in association with a stabilizing agent such as a carbohydrate. In hybridizations, the probe may be employed in an aqueous solution containing salts (e.g., NaCl), detergents or surfactants (e.g., SDS) and other components (e.g., Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% or greater free from other components with which they are naturally associated.

The term “sample”, or “biological sample”, is meant to be interpreted in its broadest sense. A biological sample suspected of containing nucleic acid encoding HGPRBMY6 protein, or fragments thereof, or HGPRBMY6 protein itself, may comprise a body fluid, an extract from cells or tissue, chromosomes isolated from a cell (e.g., a spread of metaphase chromosomes), organelle, or membrane isolated from a cell, a cell, nucleic acid such as genomic DNA (in solution or bound to a solid support such as for Southern analysis), RNA (in solution or bound to a solid support such as for Northern analysis), cDNA (in solution or bound to a solid support), a tissue, a tissue print and the like.

“Transformation” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “mimetic” refers to a molecule, the structure of which is developed from knowledge of the structure of HGPRBMY6 protein, or portions thereof, and as such, is able to effect some or all of the actions of HGPRBMY6 protein.

The term “portion” with regard to a protein (as in “a portion of a given protein”) refers to fragments or segments of that protein. The fragments may range in size from four or five amino acid residues to the entire amino acid sequence minus one amino acid. Thus, a protein “comprising at least a portion of the amino acid sequence of SEQ ID NO:3” encompasses the full-length human HGPRBMY6 polypeptide, and fragments thereof.

The term “antibody” refers to intact molecules as well as fragments thereof, such as Fab, F(ab′)₂, Fv, which are capable of binding an epitopic or antigenic determinant. Antibodies that bind to HGPRBMY6 polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (e.g., a mouse, a rat, or a rabbit).

The term “humanized” antibody refers to antibody molecules in which amino acids have been replaced in the non-antigen binding regions in order to more closely resemble a human antibody, while still retaining the original binding capability, e.g., as described in U.S. Pat. No. 5,585,089 to C. L. Queen et al.

The term “antigenic determinant” refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide and a binding molecule, such as an agonist, an antagonist, or an antibody. The interaction is dependent upon the presence of a particular structure (i.e., an antigenic determinant or epitope) of the protein that is recognized by the binding molecule. For example, if an antibody is specific for epitope “A”, the presence of a protein containing epitope A (or free, unlabeled A) in a reaction containing labeled “A” and the antibody will reduce the amount of labeled A bound to the antibody.

The term “correlates with expression of a polynucleotide” indicates that the detection of the presence of ribonucleic acid that is similar to SEQ ID NO:1 by Northern analysis is indicative of the presence of mRNA encoding HGPRBMY6 polypeptide (SEQ ID NO:2) in a sample and thereby correlates with expression of the transcript from the polynucleotide encoding the protein.

An “alteration” in the polynucleotide of SEQ ID NO: 1 comprises any alteration in the sequence of the polynucleotides encoding the HGPRBMY6 polypeptide (SEQ ID NO:2), including deletions, insertions, and point mutations that may be detected using hybridization assays. Included within this definition is the detection of alterations to the genomic DNA sequence which encodes the HGPRBMY6 polypeptide (e.g., by alterations in the pattern of restriction fragment length polymorphisms capable of hybridizing to SEQ ID NO:2), the inability of a selected fragment of the polypeptide of SEQ ID NO:2 to hybridize to a sample of genomic DNA (e.g., using allele-specific oligonucleotide probes), and improper or unexpected hybridization, such as hybridization to a locus other than the normal chromosomal locus for the polynucleotide sequence encoding the HGPRBMY6 polypeptide (e.g., using fluorescent in situ hybridization (FISH) to metaphase chromosome spreads).

### DESCRIPTION OF THE PRESENT INVENTION

The present invention provides a novel human member of the G-protein coupled receptor (GPCR) family (HGPRBMY6). Based on sequence homology, the protein HGPRBMY6 is a novel human GPCR. This protein sequence has been predicted to contain seven transmembrane domains which is a characteristic structural feature of GPCRs. HGPRBMY6 is related to latrophilin, alpha-latrotoxin, and CL3 receptors based on sequence similarity. This orphan GPCR is expressed highly in small intestine and colonic tissues. HGPRBMY6 polypeptides and polynucleotides are useful for diagnosing diseases related to over- and under-expression of HGPRBMY6 proteins by identifying mutations in the HGPRBMY6 gene using HGPRBMY6 probes, or determining HGPRBMY6 protein or mRNA expression levels. HGPRBMY6 polypeptides are also useful for screening compounds, which affect activity of the protein. The invention encompasses the polynucleotide encoding the HGPRBMY6 polypeptide and the use of the HGPRBMY6 polynucleotide or polypeptide, or composition in thereof, the screening, diagnosis, treatment, or prevention of disorders associated with aberrant or uncontrolled cellular growth and/or function, such as intestinal bowel disorders, neoplastic diseases (e.g., cancers and tumors), with particular regard to those diseases or disorders related to the small intestine and colon, e.g. intestinal bowel disorders, in addition to immune, cardiovascular, and neurological disorders. More specifically, diseases that can be treated with HGPRBMY6 include intestinal bowel disorders, pain, anorexia, HIV infections, cancers, bulimia, asthma, Parkinson's disease, acute heart failure, hypotension, hypertension, osteoporosis, angina pectoris, myocardial infarction, psychotic, immune, metabolic, cardiovascular and neurological disorders.

Nucleic acids encoding human HGPRBMY6 according to the present invention were first identified in Incyte CloneID: 2206642 from a library obtained from fetal small intestine tissue through a computer search for amino acid sequence alignments (see Example 1).

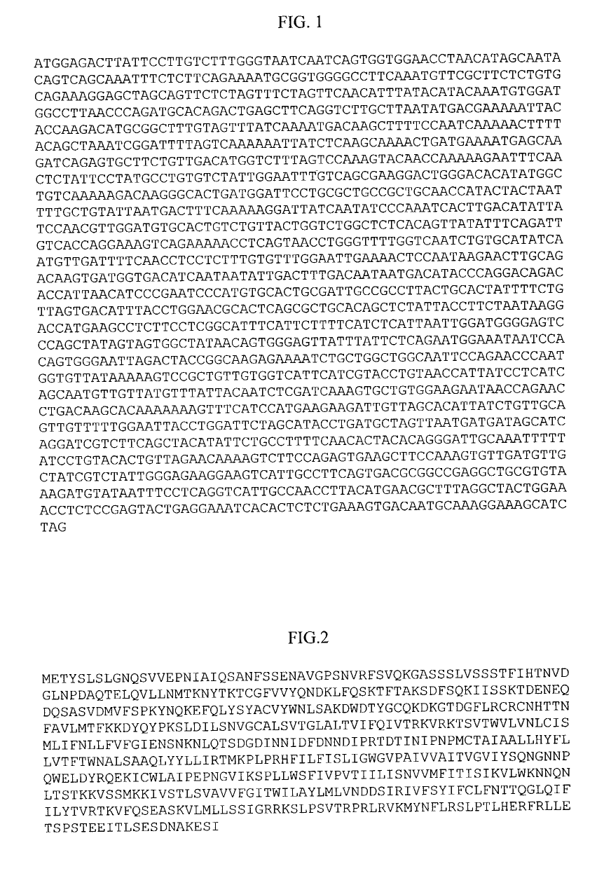

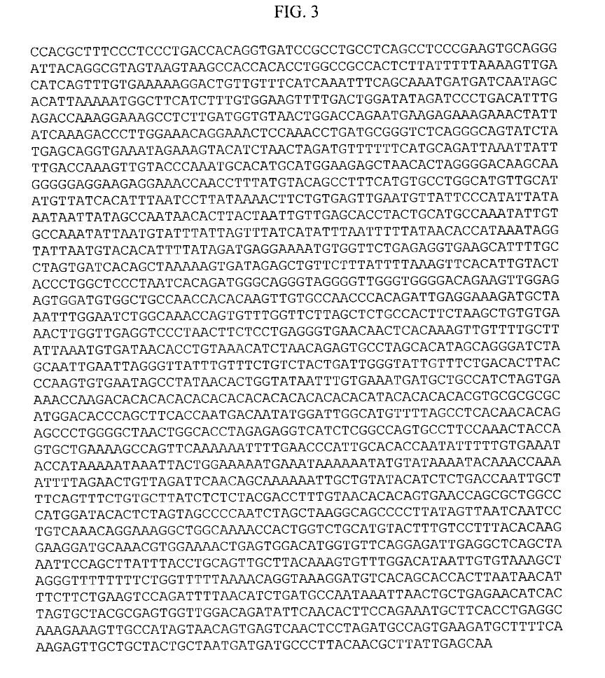

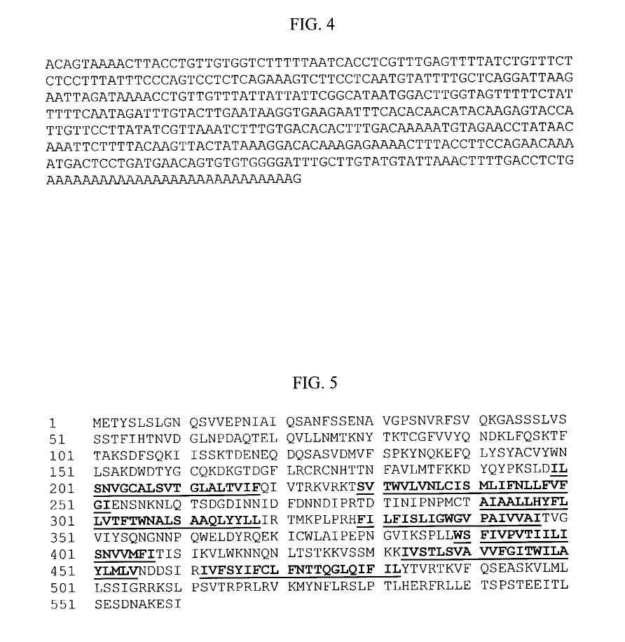

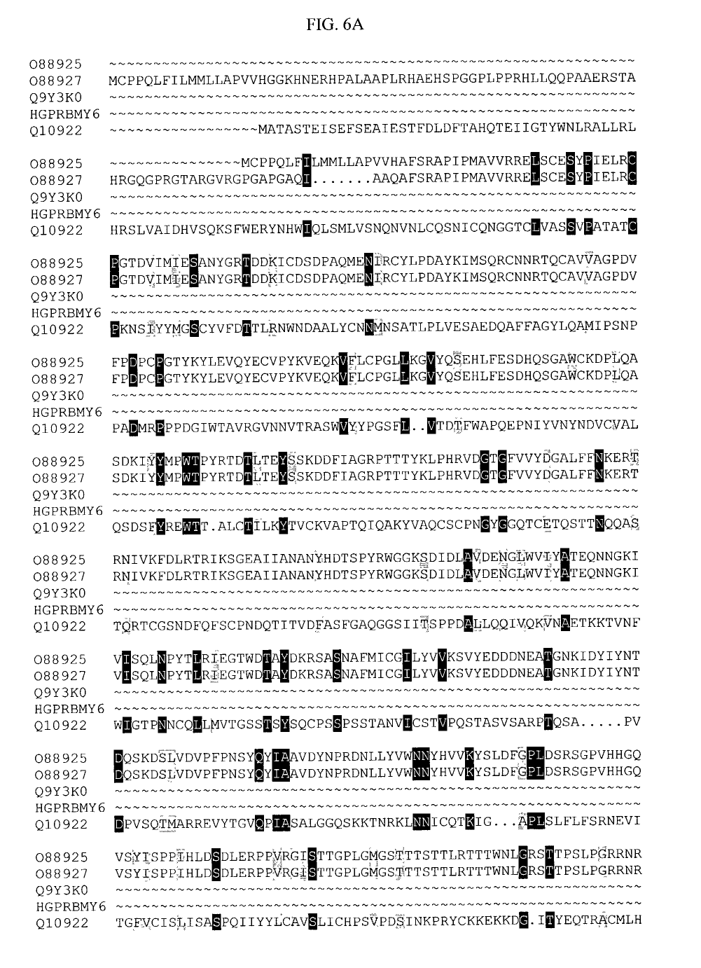

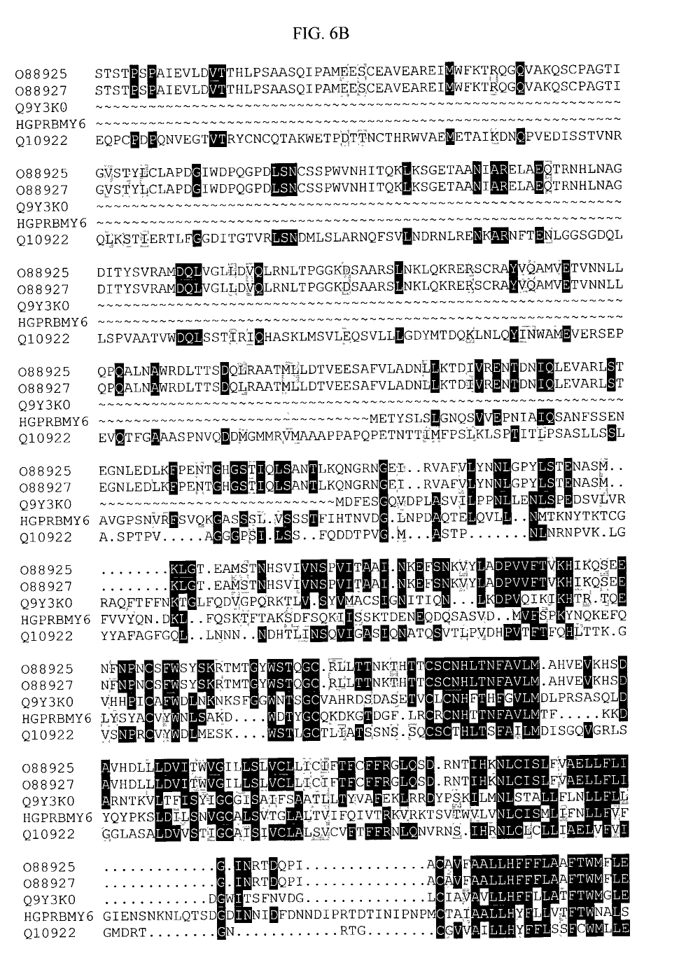

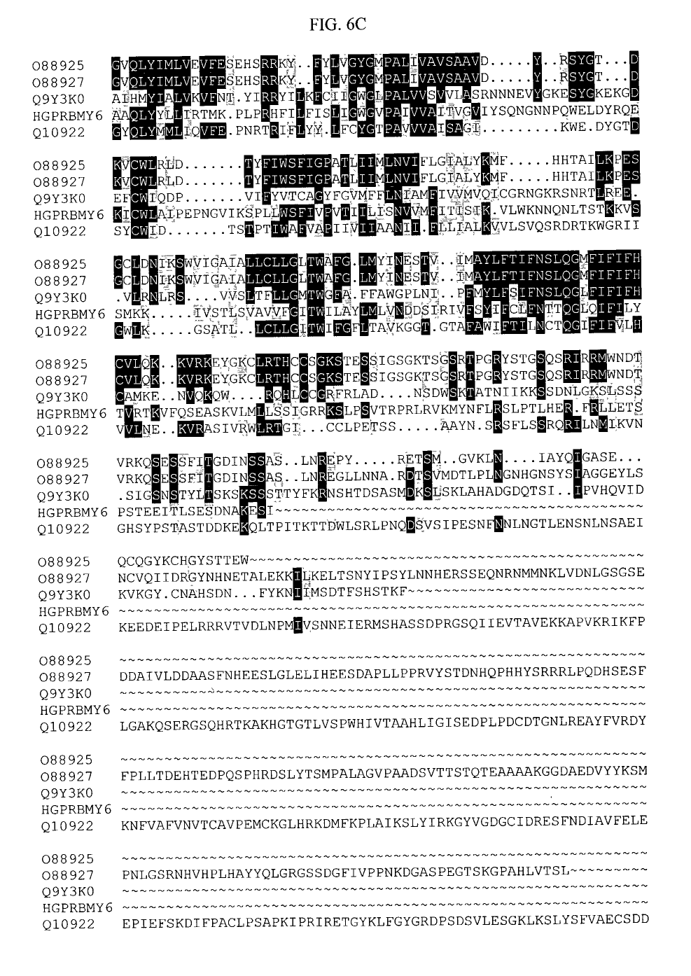

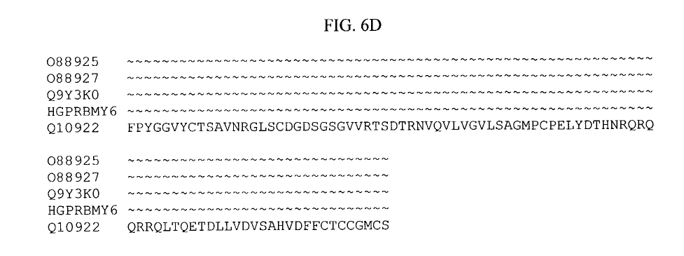

In one of its embodiments, the present invention encompasses a polypeptide comprising the amino acid sequence of SEQ ID NO:2 as shown in FIG. 1. The HGPRBMY6 polypeptide is 560 amino acids in length and shares amino acid sequence homology with the putative novel seven transmembrane domain protein, DJ287G14.2 (Acc. No.:Q9Y3KO). The HGPRBMY6 polypeptide shares 27.5% identity and 39.2% similarity with 363 amino acids of the human putative novel seven transmembrane domain protein, DJ287G14.2, wherein “similar” amino acids are those which have the same/similar physical properties and in many cases, the function is conserved with similar residues. The HGPRBMY6 polypeptide shares 30.6% identity and 41.7% similarity with the rattus norvegicus (Norway rat) CL3AB (Acc. No.:088925); 30.6% identity and 41.9% similarity with the rattus norvegicus calcium-independent alpha-latrotoxin receptor 3 precursor (LRP3; Acc. No.:088927); and 29.3% identity and 39.1% similarity with the caenorhabditis elegans hypothetical 174.3 KD protein B0286.2 in chromosome II (Acc. No.:Q10922).

Variants of the HGPRBMY6 polypeptide are also encompassed by the present invention. A preferred HGPRBMY6 variant has at least 75 to 80%, more preferably at least 85 to 90%, and even more preferably at least 90% amino acid sequence identity to the amino acid sequence claimed herein, and which retains at least one biological, immunological, or other functional characteristic or activity of HGPRBMY6 polypeptide. Most preferred is a variant having at least 95% amino acid sequence identity to that of SEQ ID NO:2.

In another embodiment, the present invention encompasses polynucleotides, which encode HGPRBMY6 polypeptide. Accordingly, any nucleic acid sequence, which encodes the amino acid sequence of HGPRBMY6 polypeptide, can be used to produce recombinant molecules that express HGPRBMY6 protein. In a particular embodiment, the present invention encompasses the HGPRBMY6 polynucleotide comprising the nucleic acid sequence of SEQ ID NO:2 and as shown in FIG. 1. More particularly, the present invention provides the HGPRBMY6 clone, deposited at the American Type Culture Collection (ATCC), 10801 University Boulevard, Manassas, Va. 20110-2209 on Nov. 15, 2000 and under ATCC Accession No. PTA-2677 according to the terms of the Budapest Treaty.

As will be appreciated by the skilled practitioner in the art, the degeneracy of the genetic code results in the production of a multitude of nucleotide sequences encoding HGPRBMY6 polypeptide. Some of the sequences bear minimal homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring HGPRBMY6, and all such variations are to be considered as being specifically disclosed.

Although nucleotide sequences which encode the HGPRBMY6 polypeptide and its variants are preferably capable of hybridizing to the nucleotide sequence of the naturally occurring HGPRBMY6 polypeptide under appropriately selected conditions of stringency, it may be advantageous to produce nucleotide sequences encoding the HGPRBMY6 polypeptide, or its derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Other reasons for substantially altering the nucleotide sequence encoding the HGPRBMY6 polypeptide, and its derivatives, without altering the encoded amino acid sequences include the production of RNA transcripts having more desirable properties, such as a greater half-life, than transcripts produced from the naturally occurring sequence.

The present invention also encompasses production of DNA sequences, or portions thereof, which encode the HGPRBMY6 polypeptide, and its derivatives, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding HGPRBMY6 polypeptide, or any fragment thereof.

Also encompassed by the present invention are polynucleotide sequences that are capable of hybridizing to the claimed nucleotide sequence of HGPRBMY6, such as that shown in SEQ ID NO: 1, under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the HGPRBMY6 polypeptide sequence of SEQ ID NO:2 and other sequences which are degenerate to those which encode HGPRBMY6 polypeptide (e.g., as a non-limiting example: prewashing solution of 2×SSC, 0.5% SDS, 1.0 mM EDTA, pH 8.0, and hybridization conditions of 50° C., 5×SSC, overnight.

The nucleic acid sequence encoding the HGPRBMY6 protein may be extended utilizing a partial nucleotide sequence and employing various methods known in the art to detect upstream sequences such as promoters and regulatory elements. For example, one method, which may be employed, is restriction-site PCR, which utilizes universal primers to retrieve unknown sequence adjacent to a known locus (G. Sarkar, 1993, PCR Methods Applic., 2:318-322). In particular, genomic DNA is first amplified in the presence of primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences Inc., Plymouth, Minn.), or another appropriate program, to be 22-30 nucleotides in length, to have a GC content of 50% or more, and to anneal to the target sequence at temperatures about 68°-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. In addition, PCR, nested primers, and PROMOTERFINDER libraries can be used to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, since they will contain more sequences, which contain the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA sequencers (PE Biosystems).

Commercially available capillary electrophoresis systems may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled device camera. Output/light intensity may be converted to electrical signal using appropriate software (e.g., GENOTYPER and SEQUENCE NAVIGATOR, PE Biosystems) and the entire process—from loading of samples to computer analysis and electronic data display—may be computer controlled. Capillary electrophoresis is especially preferable for the sequencing of small pieces of DNA, which might be present in limited amounts in a particular sample.

In another embodiment of the present invention, polynucleotide sequences or fragments thereof which encode HGPRBMY6 polypeptide, or peptides thereof, may be used in recombinant DNA molecules to direct the expression of HGPRBMY6 polypeptide product, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express HGPRBMY6 protein.

As will be appreciated by those having skill in the art, it may be advantageous to produce HGPRBMY6 polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequence of the present invention can be engineered using methods generally known in the art in order to alter HGPRBMY6 polypeptide-encoding sequences for a variety of reasons, including, but not limited to, alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In another embodiment of the present invention, natural, modified, or recombinant nucleic acid sequences encoding HGPRBMY6 polypeptide may be ligated to a heterologous sequence to encode a fusion protein. For example, for screening peptide libraries for inhibitors of HGPRBMY6 activity, it may be useful to encode a chimeric HGPRBMY6 protein that can be recognized by a commercially available antibody. A fusion protein may also be engineered to contain a cleavage site located between the HGPRBMY6 protein-encoding sequence and the heterologous protein sequence, so that HGPRBMY6 protein may be cleaved and purified away from the heterologous moiety.

In another embodiment, sequences encoding HGPRBMY6 polypeptide may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the protein itself may be produced using chemical methods to synthesize the amino acid sequence of HGPRBMY6 polypeptide, or a fragment or portion thereof. For example, peptide synthesis can be performed using various solid-phase techniques (J. Y. Roberge et al., 1995, Science, 269:202-204) and automated synthesis may be achieved, for example, using the ABI 431 A Peptide Synthesizer (PE Biosystems).

The newly synthesized peptide can be substantially purified by preparative high performance liquid chromatography (e.g., T. Creighton, 1983, Proteins Structures and Molecular Principles, W. H. Freeman and Co., New York, N.Y.), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of HGPRBMY6 polypeptide or any portion thereof, may be altered during direct synthesis and/or combined using chemical methods with sequences from other proteins, or any part thereof, to produce a variant polypeptide.

To express a biologically active HGPRBMY6 polypeptide or peptide, the nucleotide sequences encoding HGPRBMY6 polypeptide, or functional equivalents, may be inserted into an appropriate expression vector, i.e., a vector, which contains the necessary elements for the transcription and translation of the inserted coding sequence.

Methods, which are well known to those skilled in the art, may be used to construct expression vectors containing sequences encoding HGPRBMY6 polypeptide and appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding HGPRBMY6 polypeptide. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., bacculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems. The host cell employed is not limiting to the present invention.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, La Jolla, Calif.) or PSPORT1 plasmid (Life Technologies), and the like, may be used. The baculovirus polyhedrin promoter may be used in insect cells. Promoters or enhancers derived from the genomes of plant cells (e.g., heat shock, RUBISCO; and storage protein genes), or from plant viruses (e.g., viral promoters or leader sequences), may be cloned into the vector. In mammalian cell systems, promoters from mammalian genes or from mammalian viruses are preferred. If it is necessary to generate a cell line that contains multiple copies of the sequence encoding HGPRBMY6, vectors based on SV40 or EBV may be used with an appropriate selectable marker.

In bacterial systems, a number of expression vectors may be selected, depending upon the use intended for the expressed HGPRBMY6 product. For example, when large quantities of expressed protein are needed for the induction of antibodies, vectors, which direct high level expression of fusion proteins that are readily purified, may be used. Such vectors include, but are not limited to, the multifunctional E. coli cloning and expression vectors such as BLUESCRIPT (Stratagene), in which the sequence encoding HGPRBMY6 polypeptide may be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) may also be used to express foreign polypeptides, as fusion proteins with glutathione S-transferase (GST). In general, such fusion proteins are soluble and can be easily purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. (For reviews, see F. M. Ausubel et al., supra, and Grant et al., 1987, Methods Enzymol., 153:516-544).

Should plant expression vectors be desired and used, the expression of sequences encoding HGPRBMY6 polypeptide may be driven by any of a number of promoters. For example, viral promoters such as the ³⁵S and 19S promoters of CaMV may be used alone or in combination with the omega leader sequence from TMV (N. Takamatsu, 1987, EMBO J., 6:307-311). Alternatively, plant promoters such as the small subunit of RUBISCO, or heat shock promoters, may be used (G. Coruzzi et al., 1984, EMBO J., 3:1671-1680; R. Broglie et al., 1984, Science, 224:838-843; and J. Winter et al., 1991, Results Probl. Cell Differ. 17:85-105). These constructs can be introduced into plant cells by direct DNA transformation or pathogen-mediated transfection. Such techniques are described in a number of generally available reviews (see, for example, S. Hobbs or L. E. Murry, In: McGraw Hill Yearbook of Science and Technology (1992) McGraw Hill, New York, N.Y.; pp. 191-196).

An insect system may also be used to express HGPRBMY6 polypeptide. For example, in one such system, Autographa californica nuclear polyhedrosis virus (AcNPV) is used as a vector to express foreign genes in Spodoptera frugiperda cells or in Trichoplusia larvae. The sequences encoding HGPRBMY6 polypeptide may be cloned into a non-essential region of the virus such as the polyhedrin gene and placed under control of the polyhedrin promoter. Successful insertion of HGPRBMY6 polypeptide will render the polyhedrin gene inactive and produce recombinant virus lacking coat protein. The recombinant viruses may then be used to infect, for example, S. frugiperda cells or Trichoplusia larvae in which the HGPRBMY6 polypeptide product may be expressed (E. K. Engelhard et al., 1994, Proc. Nat. Acad. Sci., 91:3224-3227).

In mammalian host cells, a number of viral-based expression systems may be utilized. In cases where an adenovirus is used as an expression vector, sequences encoding HGPRBMY6 polypeptide may be ligated into an adenovirus transcription/translation complex containing the late promoter and tripartite leader sequence. Insertion in a non-essential E1 or E3 region of the viral genome may be used to obtain a viable virus which is capable of expressing HGPRBMY6 polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding HGPRBMY6 polypeptide. Such signals include the ATG initiation codon and adjacent sequences. In cases where sequences encoding HGPRBMY6 polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals, including the ATG initiation codon, should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system that is used, such as those described in the literature (D. Scharf et al., 1994, Results Probl. Cell Differ., 20:125-162).

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), American Type Culture Collection (ATCC), 10801 University Boulevard, Manassas, Va. 20110-2209, and may be chosen to ensure the correct modification and processing of the foreign protein.

For long-term, high-yield production of recombinant proteins, stable expression is preferred. For example, cell lines, which stably express HGPRBMY6 protein, may be transformed using expression vectors, which may contain viral origins of replication and/or endogenous expression elements and a selectable marker gene on the same, or on a separate, vector. Following the introduction of the vector, cells may be allowed to grow for 1-2 days in an enriched cell culture medium before they are switched to selective medium. The purpose of the selectable marker is to confer resistance to selection, and its presence allows the growth and recovery of cells, which successfully express the introduced sequences. Resistant clones of stably transformed cells may be proliferated using tissue culture techniques appropriate to the cell type.

Any number of selection systems may be used to recover transformed cell lines. These include, but are not limited to, the Herpes Simplex Virus thymidine 25-kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding HGPRBMY6 polypeptide is inserted within a marker gene sequence, recombinant cells containing sequences encoding HGPRBMY6 polypeptide can be identified by the absence of marker gene function. Alternatively, a marker gene can be placed in tandem with a sequence encoding HGPRBMY6 polypeptide under the control of a single promoter. Expression of the marker gene in response to induction or selection usually indicates co-expression of the tandem gene.

Alternatively, host cells, which contain the nucleic acid, sequence encoding HGPRBMY6 polypeptide and which express HGPRBMY6 polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding HGPRBMY6 polypeptide can be detected by DNA-DNA or DNA-RNA hybridization, or by amplification using probes or portions or fragments of polynucleotides encoding HGPRBMY6 polypeptide. Nucleic acid amplification based assays involve the use of oligonucleotides or oligomers, based on the sequences encoding HGPRBMY6 polypeptide, to detect transformants containing DNA or RNA encoding HGPRBMY6 polypeptide.

A wide variety of labels and conjugation techniques are known and employed by those skilled in the art and may be used in various nucleic acid and amino acid assays. Means for producing labeled hybridization or PCR probes for detecting sequences related to polynucleotides encoding HGPRBMY6 polypeptide include oligo-labeling, nick translation, end-labeling, or PCR amplification using a labeled nucleotide. Alternatively, the sequences encoding HGPRBMY6 polypeptide, or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which may be used include radionuclides, enzymes, fluorescent, chemiluminescent, or chromogenic agents, as well as substrates, cofactors, inhibitors, magnetic particles, and the like.

Host cells transformed with nucleotide sequences encoding HGPRBMY6 protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode HGPRBMY6 protein may be designed to contain signal sequences which direct secretion of the HGPRBMY6 protein through a prokaryotic or eukaryotic cell membrane. Other constructions may be used to join nucleic acid sequences encoding HGPRBMY6 protein to nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and HGPRBMY6 protein may be used to facilitate purification. One such expression vector provides for expression of a fusion protein containing HGPRBMY6 and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying from the fusion protein. For a discussion of suitable vectors for fusion protein production, see D. J. Kroll et al., 1993; DNA Cell Biol., 12:441-453.

In addition to recombinant production, fragments of HGPRBMY6 polypeptide may be produced by direct peptide synthesis using solid-phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems). Various fragments of HGPRBMY6 polypeptide can be chemically synthesized separately and then combined using chemical methods to produce the full length molecule.

The present invention is also directed to peptides that bind to the HGPRBMY6 polypeptide of the present invention. The present invention is directed to peptides that bind to the HG polypeptide of the present invention having a polypeptide sequence selected from the group consisting of SEQ ID NO:1, 2, 3, and 4.

Human artificial chromosomes (HACs) may be used to deliver larger fragments of DNA than can be contained and expressed in a plasmid vector. HACs are linear microchromosomes which may contain DNA sequences of 10K to 10M in size, and contain all of the elements that are required for stable mitotic chromosome segregation and maintenance (see, J. J. Harrington et al., 1997, Nature Genet., 15:345-355). HACs of 6 to 10M are constructed and delivered via conventional delivery methods (e.g., liposomes, polycationic amino polymers, or vesicles) for therapeutic purposes.

### Diagnostic Assays

A variety of protocols for detecting and measuring the expression of HGPRBMY6 polypeptide using either polyclonal or monoclonal antibodies specific for the protein are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on the HGPRBMY6 polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

This invention also relates to the use of HGPRBMY6 polynucleotides as diagnostic reagents. Detection of a mutated form of the HGPRBMY6 gene associated with a dysfunction will provide a diagnostic tool that can add to or define a diagnosis of a disease or susceptibility to a disease which results from under-expression, over-expression, or altered expression of HGPRBMY6. Individuals carrying mutations in the HGPRBMY6 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Hybridizing amplified DNA to labeled HGPRBMY6 polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (see, e.g., Myers et al., Science (1985) 230:1242). Sequence changes at specific locations may also be revealed by nuclease protection assays, such as RNase and S1 protection or the chemical cleavage method. See Cotton et al., Proc. Natl. Acad. Sci., USA (1985) 85:43297-4401. In another embodiment, an array of oligonucleotides probes comprising HGPRBMY6 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, 274:610-613, 1996).

The diagnostic assays offer a process for diagnosing or determining a susceptibility to infections such as bacterial, fungal, protozoan and viral infections, particularly infections caused by HIV-1 or HIV-2 through detection of a mutation in the HGPRBMY6 gene by the methods described. The invention also provides diagnostic assays for determining or monitoring susceptibility to the following conditions, diseases, or disorders: cancers; anorexia; bulimia asthma; Parkinson's disease; acute heart failure; hypotension; hypertension; urinary retention; osteoporosis, angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome.

In addition, infections such as bacterial, protozoan and viral infections, particularly infections caused by HIV-1 or HIV-2; as well as, conditions or disorders such as pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, schizophrenia, manic depression, delirium, dementia, severe mental retardation and dyskinesias, such as Huntington's disease or Gilles dela Tourett's syndrome, gastrointestinal disorders, colon disorders, pulmonary disorders, lung disorders, reproductive disorders, and cancers, particularly colon, lung and or ovarian, can be diagnosed by methods comprising determining from a sample derived from a subject having an abnormally decreased or increased level of HGPRBMY6 polypeptide or HGPRBMY6 mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as an HGPRBMY6, in a sample derived from a host are well known to those of skill in the art. Such assay methods include radioimmunoassays, competitive-binding assays, Western Blot analysis, and ELISA assays.

In another of its aspects, the present invention relates to a diagnostic kit for a disease or susceptibility to a disease, particularly infections such as bacterial, fungal, protozoan and viral infections, particularly infections caused by HIV-1 or HIV-2; pain; cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy, and psychotic and neurological disorders, including anxiety, schizophrenia, manic depression, delirium, dementia, severe medal retardation and dyskinesias, such as Huntington's disease or Gilles dela Tourett's syndrome, gastrointestinal disorders, colon disorders, pulmonary disorders, lung disorders, reproductive disorders, and cancers, particularly colon, lung and or ovarian, which comprises:

(a) a HGPRBMY6 polynucleotide, preferably the nucleotide sequence of SEQ ID NO: 1, or a fragment thereof; or

(b) a nucleotide sequence complementary to that of (a); or

(c) a HGPRBMY6 polypeptide, preferably the polypeptide of SEQ ID NO:2, or a fragment thereof; or

(d) an antibody to a HGPRBMY6 polypeptide, preferably to the polypeptide of SEQ ID NO:2, or combinations thereof.

It will be appreciated that in any such kit, (a), (b), (c) or (d) may comprise a substantial component.

The GPCR polynucleotides which may be used in the diagnostic assays according to the present invention include oligonucleotide sequences, complementary RNA and DNA molecules, and PNAs. The polynucleotides may be used to detect and quantify HGPRBMY6-encoding nucleic acid expression in biopsied tissues in which expression (or under- or overexpression) of the HGPRBMY6 polynucleotide may be correlated with disease. The diagnostic assays may be used to distinguish between the absence, presence, and excess expression of HGPRBMY6, and to monitor regulation of HGPRBMY6 polynucleotide levels during therapeutic treatment or intervention.

In a related aspect, hybridization with PCR probes which are capable of detecting polynucleotide sequences, including genomic sequences, encoding HGPRBMY6 polypeptide, or closely related molecules, may be used to identify nucleic acid sequences which encode HGPRBMY6 polypeptide. The specificity of the probe, whether it is made from a highly specific region, e.g., about 8 to 10 contiguous nucleotides in the 5′ regulatory region, or a less specific region, e.g., especially in the 3′ coding region, and the stringency of the hybridization or amplification (maximal, high, intermediate, or low) will determine whether the probe identifies only naturally occurring sequences encoding HGPRBMY6 polypeptide, alleles thereof, or related sequences.

Probes may also be used for the detection of related sequences, and should preferably contain at least 50% of the nucleotides encoding the HGPRBMY6 polypeptide. The hybridization probes of this invention may be DNA or RNA and may be derived from the nucleotide sequence of SEQ ID NO:1, or from genomic sequence including promoter, enhancer elements, and introns of the naturally occurring HGPRBMY6 protein.

Methods for producing specific hybridization probes for DNA encoding the HGPRBMY6 polypeptide include the cloning of a nucleic acid sequence that encodes the HGPRBMY6 polypeptide, or HGPRBMY6 derivatives, into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of detector/reporter groups, e.g., radionuclides such as ³²P or ³⁵S, or enzymatic labels, such as alkaline phosphatase coupled to the probe via avidin/biotin coupling systems, and the like.

The polynucleotide sequence encoding the HGPRBMY6 polypeptide, or fragments thereof, may be used for the diagnosis of disorders associated with expression of HGPRBMY6. Examples of such disorders or conditions are described above for “Therapeutics”. The polynucleotide sequence encoding the HGPRBMY6 polypeptide may be used in Southern or Northern analysis, dot blot, or other membrane-based technologies; in PCR technologies; or in dip stick, pin, ELISA or chip assays utilizing fluids or tissues from patient biopsies to detect the status of, e.g., levels or overexpression of HGPRBMY6, or to detect altered HGPRBMY6 expression. Such qualitative or quantitative methods are well known in the art.

In a particular aspect, the nucleotide sequence encoding the HGPRBMY6 polypeptide may be useful in assays that detect activation or induction of various neoplasms or cancers, particularly those mentioned supra. The nucleotide sequence encoding the HGPRBMY6 polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the HGPRBMY6 polypeptide in the sample indicates the presence of the associated disease. Such assays may also be used to evaluate the efficacy of a particular therapeutic treatment regimen in animal studies, in clinical trials, or in monitoring the treatment of an individual patient.

To provide a basis for the diagnosis of disease associated with expression of HGPRBMY6, a normal or standard profile for expression is established. This may be accomplished by combining body fluids or cell extracts taken from normal subjects, either animal or human, with a sequence, or a fragment thereof, which encodes the HGPRBMY6 polypeptide, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject (patient) values is used to establish the presence of disease.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to cancer, the presence of an abnormal amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the nucleic acid sequence encoding the HGPRBMY6 polypeptide may involve the use of PCR. Such oligomers may be chemically synthesized, generated enzymatically, or produced from a recombinant source. Oligomers will preferably comprise two nucleotide sequences, one with sense orientation (5′→3′) and another with antisense (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of HGPRBMY6 include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or colorimetric response gives rapid quantification.

### Therapeutic Assays

HGPRBMY6 polypeptide shares homology with G-protein coupled receptors, more specifically, latrophilin, alphalatrotoxin and CL3 receptors. Because HGPRBMY6 is highly expressed in small intestine and colonic tissue, the HGPRBMY6 product may play a role in gastrointestinal disorders, and/or in cell cycle regulation, and/or in cell signaling. The HGPRBMY6 protein may be further involved in neoplastic, gastrointestinal, and neurological disorders.

In one embodiment of the present invention, the HGPRBMY6 protein may play a role in neoplastic disorders. An antagonist of HGPRBMY6 polypeptide may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary

In a related aspect, an antibody which specifically binds to HGPRBMY6 may be used directly as an antagonist or indirectly as a targeting or delivery mechanism for bringing a pharmaceutical agent to cells or tissue which express HGPRBMY6 polypeptide.

In another embodiment of the present invention, an antagonist or inhibitory agent of the HGPRBMY6 polypeptide may be administered to a subject to prevent or treat a neurological disorder. Such disorders may include, but are not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

In another embodiment of the present invention, an antagonist or inhibitory agent of the HGPRBMY6 polypeptide may be administered to an individual to prevent or treat an immunological disorder. Such disorders may include, but are not limited to, AIDS, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections and trauma.

In a preferred embodiment of the present invention, an antagonist or inhibitory agent of the HGPRBMY6 polypeptide may be administered to an individual to prevent or treat a small intestine- or colon-related disorder, particularly since HGPRBMY6 is highly expressed in the small intestine and colon. Such conditions or disorders may include, but are not limited to, intestinal bowel disorder, ulceritis, ulceritis colitis, Crohn's disease, colon cancer, psoriasis, angiodysplasias, and gastric heterotopia.

In preferred embodiments, the HGPRBMY6 polynucleotides and polypeptides, including agonists, antagonists, and fragments thereof, are useful for modulating intracellular cAMP associated signaling pathways.

In preferred embodiments, the HGPRBMY6 polynucleotides and polypeptides, including agonists, antagonists, and fragments thereof, are useful for modulating intracellular Ca²⁺ levels, modulating Ca²⁺ sensitive signaling pathways via G alpha 15, and modulating NFAT element associated signaling pathways.

In another embodiment of the present invention, an expression vector containing the complement of the polynucleotide encoding HGPRBMY6 polypeptide may be administered to an individual to treat or prevent a neoplastic disorder, including, but not limited to, the types of cancers and tumors described above.

In another embodiment of the present invention, an expression vector containing the complement of the polynucleotide encoding the HGPRBMY6 polypeptide may be administered to an individual to treat or prevent a neurological disorder, including, but not limited to, the types of disorders described above.

In yet another embodiment of the present invention, an expression vector containing the complement of the polynucleotide encoding the HGPRBMY6 polypeptide may be administered to an individual to treat or prevent an gastrointestinal disorder, including, but not limited to, the types of small intestine- or colon-related disorders described above.

In another embodiment, the proteins, antagonists, antibodies, agonists, complementary sequences, or vectors of the present invention can be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of the HGPRBMY6 polypeptide (SEQ ID NO:2) of the present invention may be produced using methods which are generally known in the art. For example, the HGPRBMY6 transfected CHO-NFAT/CRE cell lines of the present invention are useful for the identification of agonists and antagonists of the HGPRBMY6 polypeptide. Representative uses of these cell lines would be their inclusion in a method of identifying HGPRBMY6 agonists and antagonists. Preferably, the cell lines are useful in a method for identifying a compound that modulates the biological activity of the HGPRBMY6 polypeptide, comprising the steps of (a) combining a candidate modulator compound with a host cell expressing the HGPRBMY6 polypeptide having the sequence as set forth in SEQ ID NO:2; and (b) measuring an effect of the candidate modulator compound on the activity of the expressed HGPRBMY6 polypeptide. Representative vectors expressing the HGPRBMY6 polypeptide are referenced herein (e.g., pcDNA3.1 hygro™) or otherwise known in the art.

The cell lines are also useful in a method of screening for a compound that is capable of modulating the biological activity of HGPRBMY6 polypeptide, comprising the steps of: (a) determining the biological activity of the HGPRBMY6 polypeptide in the absence of a modulator compound; (b) contacting a host cell expression the HGPRBMY6 polypeptide with the modulator compound; and (c) determining the biological activity of the HGPRBMY6 polypeptide in the presence of the modulator compound; wherein a difference between the activity of the HGPRBMY6 polypeptide in the presence of the modulator compound and in the absence of the modulator compound indicates a modulating effect of the compound.

### Modulation

Characterization of the HGPRBMY6 polypeptide of the present invention using antisense oligonucleotides led to the determination that HGPRBMY6 is involved in the positive modulation of the p21 G1/G2 cell cycle check point modulatory protein as described in Example 10 herein.

HGPRBMY6 is shown to be expressed predominantly in tissues of the gastrointestinal tract. Likewise, high mRNA levels of this gene are also observed in colon tumor cell lines. Accordingly, modulators of this gene could be most useful for treating proliferative disorders of the small and large intestine, including colon cancer. Upon antisense treatment of a tumor cell line and subsequent inhibition of mRNA production of HGPRBMY6, the cell cycle inhibitors P21 and P27 were decreased. As downregulation of P21 and P27 is consistent with proliferation, it would be preferred to agonize this target with molecules which behave as activating ligands. Based on the data at hand, it is reasonable to presume that increasing activity of this signaling molecule in such a manner would induce P21 and P27 levels, resulting in the induction of differentiation and stopping cell proliferation, a favorable effect to have on a tumor in a therapeutic setting.

These results suggest that induction of HGPRBMY6 activity or expression with a modulator would induce differentiation, and stop cellular proliferation, as p21 is a cell cycle inhibitor and is known to be associated with commitment down a differentiation pathway. Numerous known drugs in clinical trials (such as, for example, cdk2 inhibitors, dna methyltransferase inhibitors) also induce p21, and have been shown to have activity in patients with cancer. Thus, p21 induction is a plausable marker of anticancer potential when a target is appropriately modulated.

In preferred embodiments, HGPRBMY6 polynucleotides and polypeptides, including fragments thereof, are useful for treating, diagnosing, and/or ameliorating cell cycle defects, disorders related to phosphorylation, disorders related to signal transduction, proliferating disorders, and/or cancers.

Moreover, HGPRBMY6 polynucleotides and polypeptides, including fragments thereof, are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

In preferred embodiments, agonists directed to HGPRBMY6 are useful for decreasing cellular proliferation, decreasing cellular proliferation in rapidly proliferating cells, increasing the number of cells in the G1 phase of the cell cycle, increasing the number of cells in the G2 phase of the cell cycle, decreasing the number of cells that progress to the S phase of the cell cycle, decreasing the number of cells that progress to the M phase of the cell cycle, modulating DNA repair, and increasing hematopoietic stem cell expansion.

HGPRBMY6 polynucleotides and polypeptides, including fragments and agonists thereof, are useful for treating, preventing, or ameliorating proliferative disorders in a patient in need of treatment, such as cancer patients, particularly patients that have proliferative immune disorders such as leukemia, lymphomas, multiple myeloma, etc.

Moreover, antagonists directed against HGPRBMY6 are useful for increasing cellular proliferation, increasing cellular proliferation in rapidly proliferating cells, decreasing the number of cells in the G1 phase of the cell cycle, decreasing the number of cells in the G2 phase of the cell cycle, increasing the number of cells that progress to the S phase of the cell cycle, increasing the number of cells that progress to the M phase of the cell cycle, and releasing cells from G1 and/or G2 phase arrest. Such antagonists would be particularly useful for transforming normal cells into immortalized cell lines, stimulating hematopoietic cells to grow and divide, increasing recovery rates of cancer patients that have undergone chemotherapy or other therapeutic regimen, by boosting their immune responses, etc. In addition, such antagonists of HGPRBMY6 would also be useful for regenerating neural tissues (e.g., treatment of Parkinson's or Alzheimer's patients with neural stem cells, or neural cells which have been activated by an HGPRBMY6 antagonist).

Additional uses for these cell lines are described herein or otherwise known in the art. In particular, purified HGPRBMY6 protein, or fragments thereof, can be used to produce antibodies, or to screen libraries of pharmaceutical agents, to identify those which specifically bind HGPRBMY6.

Antibodies specific for HGPRBMY6 polypeptide, or immunogenic peptide fragments thereof, can be generated using methods that have long been known and conventionally practiced in the art. Such antibodies may include, but are not limited to, polyclonal, monoclonal, chimeric, single chain, Fab fragments, and fragments produced by an Fab expression library. Neutralizing antibodies, (i.e., those which inhibit dimer formation) are especially preferred for therapeutic use.

The present invention also encompasses the polypeptide sequences that intervene between each of the predicted HGPRBMY6 transmembrane domains. Since these regions are solvent accessible either extracellularly or intracellularly, they are particularly useful for designing antibodies specific to each region. Such antibodies may be useful as antagonists or agonists of the HGPRBMY6 full-length polypeptide and may modulate its activity.

The following serve as non-limiting examples of peptides or fragments that may be used to generate antibodies:

| | |
|---|---|
| METYSLSLGNQSVVEPNIAIQSANFSSENAVGPSNVRFSVQKGASSSLV | (SEQ ID NO:12) |
| SSSTFIHTNVDGLNPDAQTELQVLLNMTKNYTKTCGFVVYQNDKLFQSKTFT | |
| AKSDFSQKIISSKTDENEQDQSASVDMVFSPKYNQKEFQLYSYACVYWNLSA | |
| KDWDTYGCQKDKGTDGFLRCRCNHTTNFAVLMTFKKDYQYPKSLD, | |
| QIVTRKVRKT, | (SEQ ID NO:13) |
| ENSNKNLQTSDGDLNNIDFDNNDIPRTDTINIPNPMCT, | (SEQ ID NO:14) |
| IRTMKPLPRH, | (SEQ ID NO:15) |
| TVGVIYSQNGNNPQWELDYRQEKICWLAIPEPNGVIKSPLL, | (SEQ ID NO:16) |
| TISIKVLWKNNQNLTSTKKVSSMKK, | (SEQ ID NO:17) |
| NDDSIR, and/or | (SEQ ID NO:18) |
| YTVRTKVFQSEASKVLMLLSSIGRRKSLPSVTRPRLRVKMYNFLRSLPTLHER | (SEQ ID NO:19) |
| FRLLETSPSTEEITLSESDNAKESI. | |

In preferred embodiments, the following N-terminal HGPRBMY6 N-terminal fragment deletion polypeptides are encompassed by the present invention: M1-D198, E2-D198, T3-D198, Y4-D198, S5-D198, L6-D198, S7-D198, L8-D198, G9-D198, N10-D198, Q11-D198, S12-D198, V13-D198, V14-D198, E15-D198, P16-D198, N17-D198, I18-D198, A19-D198, I20-D198, Q21-D198, S22-D198, A23-D198, N24-D198, F25-D198, S26-D198, S27-D198, E28-D198, N29-D198, A30-D198, V31-D198, G32-D198, P33-D198, S34-D198, N35-D198, V36-D198, R37-D198, F38-D198, S39-D198, V40-D198, Q41-D198, K42-D198, G43-D198, A44-D198, S45-D198, S46-D198, S47-D198, L48-D198, V49-D198, S50-D198, S51-D198, S52-D198, T53-D198, F54-D198, I55-D198, H56-D198, T57-D198, N58-D198, V59-D198, D60-D198, G61-D198, L62-D198, N63-D198, P64-D198, D65-D198, A66-D198, Q67-D198, T68-D198, E69-D198, L70-D198, Q71-D198, V72-D198, L73-D198, L74-D198, N75-D198, M76-D198, T77-D198, K78-D198, N79-D198, Y80-D198, T81-D198, K82-D198, T83-D198, C84-D198, G85-D198, F86-D198, V87-D198, V88-D198, Y89-D198, Q90-D198, N91-D198, D92-D198, K93-D198, L94-D198, F95-D198, Q96-D198, S97-D198, K98-D198, T99-D198, F100-D198, T101-D198, A102-D198, K103-D198, S104-D198, D105-D198, F106-D198, S107-D198, Q108-D198, K109-D198, I110-D198, I111-D198, S112-D198, S113-D198, K114-D198, T115-D198, D116-D198, E117-D198, N118-D198, E119-D198, Q120-D198, D121-D198, Q122-D198, S123-D198, A124-D198, S125-D198, V126-D198, D127-D198, M128-D198, V129-D198, F130-D198, S131-D198, P132-D198, K133-D198, Y134-D198, N135-D198, Q136-D198, K137-D198, E138-D198, F139-D198, Q140-D198, L141-D198, Y142-D198, S143-D198, Y144-D198, A145-D198, C146-D198, V147-D198, Y148-D198, W149-D198, N150-D198, L151-D198, S152-D198, A153-D198, K154-D198, D155-D198, W156-D198, D157-D198, T158-D198, Y159-D198, G160-D198, C161-D198, Q162-D198, K163-D198, D164-D198, K165-D198, G166-D198, T167-D198, D168-D198, G169-D198, F170-D198, L171-D198, R172-D198, C173-D198, R174-D198, C175-D198, N176-D198, H177-D198, T178-D198, T179-D198, N180-D198, F181-D198, A182-D198, V183-D198, L184-D198, M185-D198, T186-D198, F187-D198, K188-D198, K189-D198, D190-D198, Y191-D198, and/or Q192-D198 of SEQ ID NO: 12. Polynucleotide sequences encoding these polypeptides are also provided. The present invention also encompasses the use of these N-terminal HGPRBMY6 N-terminal fragment deletion polypeptides as immunogenic and/or antigenic epitopes as described elsewhere herein.

In preferred embodiments, the following C-terminal HGPRBMY6 N-terminal fragment deletion polypeptides are encompassed by the present invention: M1-D198, M1-L197, M1-S196, M1-K195, M1-P194, M1-Y193, M1-Q192, M1-Y191, M1-D190, M1-K189, M1-K188, M1-F187, M1-T186, M1-M185, M1-L184, M1-V183, M1-A182, M1-F181, M1-N180, M1-T179, M1-T178, M1-H177, M1-N176, M1-C175, M1-R174, M1-C173, M1-R172, M1-L171, M1-F170, M1-G169, M1-D168, M1-T167, M1-G166, M1-K165, M1-D164, M1-K163, M1-Q162, M1-C161, M1-G160, M1-Y159, M1-T158, M1-D157, M1-W156, M1-D155, M1-K154, M1-A153, M1-S152, M1-L151, M1-N150, M1-W149, M1-Y148, M1-V147, M1-C146, M1-A145, M1-Y144, M1-S143, M1-Y142, M1-L141, M1-Q140, M1-F139, M1-E138, M1-K137, M1-Q136, M1-N135, M1-Y134, M1-K133, M1-P132, M1-S131, M1-F130, M1-V129, M1-M128, M1-D127, M1-V126, M1-S125, M1-A124, M1-S123, M1-Q122, M1-D121, M1-Q120, M1-E119, M1-N118, M1-E117, M1-D116, M1-T115, M1-K114, M1-S113, M1-S112, M1-I111, M1-I110, M1-K109, M1-Q108, M1-S107, M1-F106, M1-D105, M1-S104, M1-K103, M1-A102, M1-T100, M1-F100, M1-T99, M1-K98, M1-S97, M1-Q96, M1-F95, M1-L94, M1-K93, M1-D92, M1-N91, M1-Q90, M1-Y89, M1-V88, M1-V87, M1-F86, M1-G85, M1-C84, M1-T83, M1-K82, M1-T81, M1-Y80, M1-N79, M1-K78, M1-T77, M1-M76, M1-N75, M1-L74, M1-L73, M1-V72, M1-Q71, M1-L70, M1-E69, M1-T68, M1-Q67, M1-A66, M1-D65, M1-P64, M1-N63, M1-L62, M1-G61, M1-D60, M1-V59, M1-N58, M1-T57, M1-H56, M1-155, M1-F54, M1-T53, M1-S52, M1-S51, M1-S50, M1-V49, M1-L48, M1-S47, M1-S46, M1-S45, M1-A44, M1-G43, M1-K42, M1-Q41, M1-V40, M1-S39, M1-F38, M1-R37, M1-V36, M1-N35, M1-S34, M1-P33, M1-G32, M1-V31, M1-A30, M1-N29, M1-E28, M1-S27, M1-S26, M1-F25, M1-N24, M1-A23, M1-S22, M1-Q21, M1-I20, M1-A19, M1-I18, M1-N17, M1-P16, M1-E15, M1-V14, M1-V13, M1-S12, M1-Q11, M1-N10, M1-G9, M1-L8, and/or M1-S7 of SEQ ID NO:12. Polynucleotide sequences encoding these polypeptides are also provided. The present invention also encompasses the use of these C-terminal HGPRBMY6 N-terminal fragment deletion polypeptides as immunogenic and/or antigenic epitopes as described elsewhere herein.

In preferred embodiments, the following N-terminal HGPRBMY6 TM1-2 intertransmembrane domain deletion polypeptides are encompassed by the present invention: Q1-T10, I2-T10, V3-T10, and/or T4-T10 of SEQ ID NO:13. Polynucleotide sequences encoding these polypeptides are also provided. The present invention also encompasses the use of these N-terminal HGPRBMY6 TM1-2 intertransmembrane domain deletion polypeptides as immunogenic and/or antigenic epitopes as described elsewhere herein.

In preferred embodiments, the following C-terminal HGPRBMY6 TM1-2 intertransmembrane domain deletion polypeptides are encompassed by the present invention: Q1-T10, Q1-K9, Q1-R8, and/or Q1-V7 of SEQ ID NO:13. Polynucleotide sequences encoding these polypeptides are also provided. The present invention also encompasses the use of these C-terminal HGPRBMY6 TM1-2 intertransmembrane domain deletion polypeptides as immunogenic and/or antigenic epitopes as described elsewhere herein.

In preferred embodiments, the following N-terminal HGPRBMY6 TM2-3 intertransmembrane domain deletion polypeptides are encompassed by the present invention: E1-T38, N2-T38, S3-T38, N4-T38, K5-T38, N6-T38, L7-T38, Q8-T38, T9-T38, S10-T38, D11-T38, G12-T38, D13-T38, I14-T38, N15-T38, N16-T38, I17-T38, D18-T38, F19-T38, D20-T38, N21-T38, N22-T38, D23-T38, I24-T38, P25-T38, R26-T38, T27-T38, D28-T38, T29-T38, I30-T38, N31-T38, and/or 132-T38 of SEQ ID NO: 14. Polynucleotide sequences encoding these polypeptides are also provided. The present invention also encompasses the use of these N-terminal HGPRBMY6 TM2-3 intertransmembrane domain deletion polypeptides as immunogenic and/or antigenic epitopes as described elsewhere herein.

In preferred embodiments, the following C-terminal HGPRBMY6 TM2-3 intertransmembrane domain deletion polypeptides are encompassed by the present invention: E1-T38, E1-C37, E1-M36, E1-P35, E1-N34, E1-P33, E1-132, E1-N31, E1-130, E1-T29, E1-D28, E1-T27, E1-R26, E1-P25, E1-124, E1-D23, E1-N22, E1-N21, E1-D20, E1-F19, E1-D18, E1-117, E1-N16, E1-N15, E1-I14, E1-D13, E1-G12, E1-D11, E1-S10, E1-T9, E1-Q8, and/or E1-L7 of SEQ ID NO:14. Polynucleotide sequences encoding these polypeptides are also provided. The present invention also encompasses the use of these C-terminal HGPRBMY6 TM2-3 intertransmembrane domain deletion polypeptides as immunogenic and/or antigenic epitopes as described elsewhere herein.

In preferred embodiments, the following N-terminal HGPRBMY6 TM3-4 intertransmembrane domain deletion polypeptides are encompassed by the present invention: I1-H10, R2-H10, T3-H10, and/or M4-H10 of SEQ ID NO:15. Polynucleotide sequences encoding these polypeptides are also provided. The present invention also encompasses the use of these N-terminal HGPRBMY6 TM3-4 intertransmembrane domain deletion polypeptides as immunogenic and/or antigenic epitopes as described elsewhere herein.

In preferred embodiments, the following C-terminal HGPRBMY6 TM3-4 intertransmembrane domain deletion polypeptides are encompassed by the present invention: I1-H10, I1-R9, I1-P8, and/or I1-L7 of SEQ ID NO:15. Polynucleotide sequences encoding these polypeptides are also provided. The present invention also encompasses the use of these C-terminal HGPRBMY6 TM3-4 intertransmembrane domain deletion polypeptides as immunogenic and/or antigenic epitopes as described elsewhere herein.

In preferred embodiments, the following N-terminal HGPRBMY6 TM4-5 intertransmembrane domain deletion polypeptides are encompassed by the present invention: T1-L41, V2-L41, G3-L41, V4-L41, I5-L41, Y6-L41, S7-L41, Q8-L41, N9-L41, G10-L41, N11-L41, N12-L41, P13-L41, Q14-L41, W15-41, E16-L41, L17-L41, D18-L41, Y19-L41, R20-L11, Q21-L41, E22-L41, K23-L41, I24-41, C25-L41, W26-L41, L27-L41, A28-L41, I29-L41, P30-L41, E31-L41, P32-L41, N33-L41, G34-L41, and/or V35-L41 of SEQ ID NO:16. Polynucleotide sequences encoding these polypeptides are also provided. The present invention also encompasses the use of these N-terminal HGPRBMY6 TM4-5 intertransmembrane domain deletion polypeptides as immunogenic and/or antigenic epitopes as described elsewhere herein.

In preferred embodiments, the following C-terminal HGPRBMY6 TM4-5 intertransmembrane domain deletion polypeptides are encompassed by the present invention: T1-L41, T1-L40, T1-P39, T1-S38, T1-K37, T1-I36, T1-V35, T1-G34, T1-N33, T1-P32, T1-E31, T1-P30, T1-129, T1-A28, T1-L27, T1-W26, T1-C25, T1-I24, T1-K23, T1-E22, T1-Q21, T1-R20, T1-Y19, T1-D18, T1-L17, T1-E16, T1-W15, T1-Q14, T1-P13, T1-N12, T1-N11, T1-G10, T1-N9, T1-Q8, and/or T1-S7 of SEQ ID NO:16. Polynucleotide sequences encoding these polypeptides are also provided. The present invention also encompasses the use of these C-terminal HGPRBMY6 TM4-5 intertransmembrane domain deletion polypeptides as immunogenic and/or antigenic epitopes as described elsewhere herein.

In preferred embodiments, the following N-terminal HGPRBMY6 TM5-6 intertransmembrane domain deletion polypeptides are encompassed by the present invention: T1-K25, I2-K25, S3-K25, I4-K25, K5-K25, V6-K25, L7-K25, W8-K25, K9-K25, N10-K25, N11-K25, Q12-K25, N13-K25, L14-K25, T15-K25, S16-K25, T17-K25, K18-K25, and/or K19-K25 of SEQ ID NO:16. Polynucleotide sequences encoding these polypeptides are also provided. The present invention also encompasses the use of these N-terminal HGPRBMY6 TM5-6 intertransmembrane domain deletion polypeptides as immunogenic and/or antigenic epitopes as described elsewhere herein.

In preferred embodiments, the following C-terminal HGPRBMY6 TM5-6 intertransmembrane domain deletion polypeptides are encompassed by the present invention: T1-K25, T1-K24, T1-M23, T1-S22, T1-S21, T1-V20, T1-K19, T1-K18, T1-T17, T1-S16, T1-T15, T1-L14, T1-N13, T1-Q12, T1-N11, T1-N10, T1-K9, T1-W8, and/or T1-L7 of SEQ ID NO:16. Polynucleotide sequences encoding these polypeptides are also provided. The present invention also encompasses the use of these C-terminal HGPRBMY6 TM5-6 intertransmembrane domain deletion polypeptides as immunogenic and/or antigenic epitopes as described elsewhere herein.

In preferred embodiments, the following N-terminal HGPRBMY6 C-terminal fragment deletion polypeptides are encompassed by the present invention: Y1-I78, T2-I78, V3-I78, R4-I78, T5-I78, K6-I78, V7-I78, F8-I78, Q9-I78, S10-I78, E1-I78, A12-I78, S13-I78, K14-I78, V15-I78, L16-I78, M17-I78, L18-I78, L19-I78, S20-I78, S21-I78, I22-I78, G23-I78, R24-I78, R25-I78, K26-I78, S27-I78, L28-I78, P29-I78, S30-I78, V31-I78, T32-I78, R33-I78, P34-I78, R35-I78, L36-I78, R37-I78, V38-I78, K39-I78, M40-I78, Y41-I78, N42-I78, F43-I78, L44-I78, R45-I78, S46-I78, L47-I78, P48-I78, T49-I78, L50-I78, H51-I78, E52-I78, R53-I78, F54-I78, R55-I78, L56-I78, L57-I78, E58-I78, T59-I78, S60-I78, P61-I78, S62-I78, T63-I78, E64-I78, E65-I78, 166-I78, T67-I78, L68-I78, S69-I78, E70-I78, S71-I78, and/or D72-I78 of SEQ ID NO:19. Polynucleotide sequences encoding these polypeptides are also provided. The present invention also encompasses the use of these N-terminal HGPRBMY6 C-terminal fragment deletion polypeptides as immunogenic and/or antigenic epitopes as described elsewhere herein.

In preferred embodiments, the following C-terminal HGPRBMY6 C-terminal fragment deletion polypeptides are encompassed by the present invention: Y1-I78, Y1-S77, Y1-E76, Y1-K75, Y1-A74, Y1-N73, Y1-D72, Y1-S71, Y1-E70, Y1-S69, Y1-L68, Y1-T67, Y1-166, Y1-E65, Y1-E64, Y1-T63, Y1-S62, Y1-P61, Y1-S60, Y1-T59, Y1-E58, Y1-L57, Y1-L56, Y1-R55, Y1-F54, Y1-R53, Y1-E52, Y1-H51, Y1-L50, Y1-T49, Y1-P48, Y1-L47, Y1-S46, Y1-R45, Y1-L44, Y1-F43, Y1-N42, Y1-Y41, Y1-M40, Y1-K39, Y1-V38, Y1-R37, Y1-L36, Y1-R35, Y1-P34, Y1-R33, Y1-T32, Y1-V31, Y1-S30, Y1-P29, Y1-L28, Y1-S27, Y1-K26, Y1-R25, Y1-R24, Y1-G23, Y1-122, Y1-S21, Y1-S20, Y1-L19, Y1-L18, Y1-M17, Y1-L16, Y1-V15, Y1-K14, Y1-S13, Y1-A12, Y1-E11, Y1-S10, Y1-Q9, Y1-F8, and/or Y1-V7 of SEQ ID NO: 19. Polynucleotide sequences encoding these polypeptides are also provided. The present invention also encompasses the use of these C-terminal HGPRBMY6 C-terminal fragment deletion polypeptides as immunogenic and/or antigenic epitopes as described elsewhere herein.

The HGPRBMY6 polypeptides of the present invention were determined to comprise several phosphorylation sites based upon the Motif algorithm (Genetics Computer Group, Inc.). The phosphorylation of such sites may regulate some biological activity of the HGPRBMY6 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the HGPRBMY6 polypeptide to associate with other polypeptides, particularly cognate ligand for HGPRBMY6, or its ability to modulate certain cellular signal pathways.

The HGPRBMY6 polypeptide was predicted to comprise fifteen PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: QSKTFTAKSDFSQ (SEQ ID NO:27), AKSDFSQKIISSK (SEQ ID NO:28), SQKIISSKTDENE (SEQ ID NO:29), VDMVFSPKYNQKE (SEQ ID NO:30), VYWNLSAKDWDTY (SEQ ID NO:31), FAVLMTFKKDYQY (SEQ ID NO:32), IFQIVTRKVRKTS (SEQ ID NO:33), FGIENSNKNLQTS (SEQ ID NO:34), YLLIRTMKPLPRH (SEQ ID NO:35), MFITISIKVLWKN (SEQ ID NO:36), NQNLTSTKKVSSM (SEQ ID NO:37), QNLTSTKKVSSMK (SEQ ID NO:38), TKKVSSMKKIVST (SEQ ID NO:39), LVNDDSIRIVFSY (SEQ ID NO:40), and/or IFILYTVRTKVFQ (SEQ ID NO:41). Polynucleotides encoding these polypeptides are also provided. The present invention also encompasses the use of the HGPRBMY6 PKC phosphorylation site polypeptides as immunogenic and/or antigenic epitopes as described elsewhere herein.

The HGPRBMY6 polypeptide was predicted to comprise nine casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to aminoacyl-transfer RNA synthetases class-II domains may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: SLGNQSVVEPNIAI (SEQ ID NO:42), STFIHTNVDGLNPD (SEQ ID NO:43), QKIISSKTDENEQD (SEQ ID NO:44), VYWNLSAKDWDTYG (SEQ ID NO:45), KNLQTSDGDINNID (SEQ ID NO:46), LRSLPTLHERFRLL (SEQ ID NO:47), LETSPSTEEITLSE (SEQ ID NO:48), STEEITLSESDNAK (SEQ ID NO:49), and/or EEITLSESDNAKES (SEQ ID NO:50). Polynucleotides encoding these polypeptides are also provided. The present invention also encompasses the use of this casein kinase II phosphorylation site polypeptide as an immunogenic and/or antigenic epitope as described elsewhere herein.

The HGPRBMY6 polypeptide was predicted to comprise three cAMP- and cGMP-dependent protein kinase phosphorylation site using the Motif algorithm (Genetics Computer Group, Inc.). There has been a number of studies relative to the specificity of cAMP- and cGMP-dependent protein kinases. Both types of kinases appear to share a preference for the phosphorylation of serine or threonine residues found close to at least two consecutive N-terminal basic residues.

A consensus pattern for cAMP- and cGMP-dependent protein kinase phosphorylation sites is as follows: [RK](2)-x-[ST], wherein “x” represents any amino acid, and S or T is the phosphorylation site.

Additional information specific to cAMP- and cGMP-dependent protein kinase phosphorylation sites may be found in reference to the following publication: Fremisco J. R., Glass D. B., Krebs E. G, J. Biol. Chem. 255:4240-4245(1980); Glass D. B., Smith S. B., J. Biol. Chem. 258:14797-14803(1983); and Glass D. B., El-Maghrabi M. R., Pilkis S. J., J. Biol. Chem. 261:2987-2993(1986); which is hereby incorporated herein in its entirety.

In preferred embodiments, the following cAMP- and cGMP-dependent protein kinase phosphorylation site polypeptides are encompassed by the present invention: VTRKVRKTSVTWVL (SEQ ID NO:51), NLTSTKKVSSMKKI (SEQ ID NO:52), and/or LSSIGRRKSLPSVT (SEQ ID NO:53). Polynucleotides encoding this polypeptide are also provided. The present invention also encompasses the use of these cAMP- and cGMP-dependent protein kinase phosphorylation site polypeptides as immunogenic and/or antigenic epitopes as described elsewhere herein.

The HGPRBMY6 polypeptide has been shown to comprise eight glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following concensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: SLSLGNQSVVEPNI (SEQ ID NO:54), AIQSANFSSENAVG (SEQ ID NO:55), LQVLLNMTKNYTKT (SEQ ID NO:56), LNMTKNYTKTCGFV (SEQ ID NO:57), ACVYWNLSAKDWDT (SEQ ID NO:58), LRCRCNHTTNFAVL (SEQ ID NO:59), WKNNQNLTSTKKVS (SEQ ID NO:60), and/or IFCLFNTTQGLQIF (SEQ ID NO:61). Polynucleotides encoding these polypeptides are also provided. The present invention also encompasses the use of these HGPRBMY6 asparagine glycosylation site polypeptide as immunogenic and/or antigenic epitopes as described elsewhere herein.

The HGPRBMY6 polypeptide was predicted to comprise five N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: FSVQKGASSSLVSSST (SEQ ID NO:62), ILSNVGCALSVTGLAL (SEQ ID NO:63), ALSVTGLALTVIFQIV (SEQ ID NO:64), LLFVFGIENSNKNLQT (SEQ ID NO:65), and/or VAITVGVIYSQNGNNP (SEQ ID NO:66). Polynucleotides encoding these polypeptides are also provided. The present invention also encompasses the use of these N-myristoylation site polypeptides as immunogenic and/or antigenic epitopes as described elsewhere herein.

G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to 1F, 2A to 2C, 4, 5A, 5B, 6 and 7, Acetylcholine, muscarinic-type, M1 to M5, Adenosine A1, A2A, A2B and A3, Adrenergic alpha-1A to -1C; alpha-2A to -2D; beta-1 to -3, Angiotensin II types I and II, Bombesin subtypes 3 and 4, Bradykinin B1 and B2, c3a and C5a anaphylatoxin, Cannabinoid CB1 and CB2, Chemokines C-C CC-CKR-1 to CC-CKR-8, Chemokines C-X-C CXC-CKR-1 to CXC-CKR-4, Cholecystokinin-A and cholecystokinin-B/gastrin, Dopamine D1 to D5, Endothelin ET-a and ET-b, fMet-Leu-Phe (fMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-IR), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors C06G4.5, C38C10.1, C43C3.2, T27D1.3 and ZC84.4, Three putative receptors encoded in the genome of cytomegalovirus: US27, US28, and UL33., ECRF3, a putative receptor encoded in the genome of herpesvirus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative concensus sequence for GPCRs comprises the conserved triplet and also spans the major part of the third transmembrane helix, and is as follows: [GSTALIVMFYWC]-[GSTANCPDE]-{EDPKRH}-x(2)-[LIVMNQGA]-x(2)-[LIVMF]-[GSTANC]-[LIVMFYWSTAC]-[DENH]-R-[FYWCSH]-x(2)-[LIVM], where “X” represents any amino acid.

Additional information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Gamier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); http://www.gcrdb.uthscsa.edu/; and http://swift.embl-heidelberg.de/7tm/.

For the production of antibodies, various hosts including goats, rabbits, sheep, rats, mice, humans, and others, can be immunized by injection with HGPRBMY6 polypeptide, or any fragment or oligopeptide thereof, which has immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guerin) and Corynebacterium parvumn.

Preferably, the peptides, fragments, or oligopeptides used to induce antibodies to HGPRBMY6 polypeptide (i.e., immunogens) have an amino acid sequence having at least five amino acids, and more preferably, at least 7-10 amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of HGPRBMY6 amino acids may be fused with those of another protein, such as KLH, and antibodies are produced against the chimeric molecule.

Monoclonal antibodies to HGPRBMY6 polypeptide, or immunogenic fragments thereof, may be prepared using any technique which provides for the production of antibody molecules by continuous cell lines in culture. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce HGPRBMY6 polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for HGPRBMY6 polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between HGPRBMY6 polypeptide and its specific antibody. A two-site, monoclonal-based immunoassay utilizing monoclonal antibodies reactive with two non-interfering HGPRBMY6 polypeptide epitopes is preferred, but a competitive binding assay may also be employed (Maddox, supra).

Another aspect of the invention relates to a method for inducing an immunological response in a mammal which comprises inoculating the mammal with HGPRBMY6 polypeptide, or a fragment thereof, adequate to produce antibody and/or T cell immune response to protect said animal from infections such as bacterial, fungal, protozoan and viral infections, particularly infections caused by HIV-1 or HIV-2. Yet another aspect of the invention relates to a method of inducing immunological response in a mammal which comprises, delivering HGPRBMY6 polypeptide via a vector directing expression of HGPRBMY6 polynucleotide in vivo in order to induce such an immunological response to produce antibody to protect said animal from diseases.

A further aspect of the invention relates to an immunological/vaccine formulation (composition) which, when introduced into a mammalian host, induces an immunological response in that mammal to an HGPRBMY6 polypeptide wherein the composition comprises an HGPRBMY6 polypeptide or HGPRBMY6 gene. The vaccine formulation may further comprise a suitable carrier. Since the HGPRBMY6 polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampoules and vials, and may be stored in a freeze-dried condition requiring only the addition of the sterile liquid carrier immediately prior to use. The vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In an embodiment of the present invention, the polynucleotide encoding the HGPRBMY6 polypeptide, or any fragment or complement thereof, may be used for therapeutic purposes. In one aspect, antisense, to the polynucleotide encoding the HGPRBMY6 polypeptide, may be used in situations in which it would be desirable to block the transcription of the mRNA. In particular, cells may be transformed with sequences complementary to polynucleotides encoding HGPRBMY6 polypeptide. Thus, complementary molecules may be used to modulate HGPRBMY6 polynucleotide and polypeptide activity, or to achieve regulation of gene function. Such technology is now well known in the art, and sense or antisense oligomers or oligonucleotides, or larger fragments, can be designed from various locations along the coding or control regions of polynucleotide sequences encoding HGPRBMY6 polypeptide.

Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors which will express a nucleic acid sequence that is complementary to the nucleic acid sequence encoding the HGPRBMY6 polypeptide. These techniques are described both in J. Sambrook et al., supra and in F. M. Ausubel et al., supra.

Polypeptides used in treatment can also be generated endogenously in the subject, in treatment modalities often referred to a “gene therapy”. Thus, for example, cells from a subject may be engineered with a polynucleotide, such as DNA or RNA, to encode a polypeptide ex vivo, and for example, by the use of a retroviral plasmid vector. The cells can then be introduced into the subject.

Transforming a cell or tissue with an expression vector that expresses high levels of an HGPRBMY6 polypeptide-encoding polynucleotide, or a fragment thereof can turn off the genes encoding the HGPRBMY6 polypeptide. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Designing antisense molecules or complementary nucleic acid can obtain modifications of gene expression sequences (DNA, RNA, or PNA), to the control, 5′, or regulatory regions of the gene encoding the HGPRBMY6 polypeptide, (e.g., signal sequence, promoters, enhancers, and introns). Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Ribozymes, i.e., enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Suitable examples include engineered hammerhead motif ribozyme molecules that can specifically and efficiently catalyze endonucleolytic cleavage of sequences encoding HGPRBMY6 polypeptide.

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes according to the invention may be prepared by any method known in the art for the synthesis of nucleic acid molecules. Such methods include techniques for chemically synthesizing oligonucleotides, for example, solid phase phosphoramidite chemical synthesis. Alternatively, RNA molecules may be generated by in vitro and in vivo transcription of DNA sequences encoding HGPRBMY6. Such DNA sequences may be incorporated into a wide variety of vectors with suitable RNA polymerase promoters such as T7 or SP. Alternatively, the cDNA constructs that constitutively or inducibly synthesize complementary RNA can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

Many methods for introducing vectors into cells or tissues are available and are equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art.

Any of the therapeutic methods described above may be applied to any individual in need of such therapy, including, for example, mammals such as dogs, cats, cows, horses, rabbits, monkeys, and most preferably, humans.

A further embodiment of the present invention embraces the administration of a pharmaceutical composition, in conjunction with a pharmaceutically acceptable carrier, diluent, or excipient, for any of the above-described therapeutic uses and effects. Such pharmaceutical compositions may comprise HGPRBMY6 nucleic acid, polypeptide, or peptides, antibodies to HGPRBMY6 polypeptide, mimetics, agonists, antagonists, or inhibitors of HGPRBMY6 polypeptide or polynucleotide. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (i.e., the HGPRBMY6 nucleic acid or polypeptide, or functional fragments thereof), the pharmaceutical compositions may contain suitable pharmaceutically acceptable carriers or excipients comprising auxiliaries which facilitate processing of the active compounds into preparations which can be used pharmaceutically. Further details on techniques for formulation and administration are provided in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents who increase the solubility of the compounds to allow for the preparation of highly concentrated solutions.

For topical or nasal administration, penetrants or permeation agents that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of HGPRBMY6 product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, e.g., using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, HGPRBMY6 polypeptide, or fragments thereof, antibodies to HGPRBMY6 polypeptide, agonists, antagonists or inhibitors of HGPRBMY6 polypeptide, which ameliorates, reduces, or eliminates the symptoms or condition. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, ED₅₀/LD₅₀. Pharmaceutical compositions, which exhibit large therapeutic indices, are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, sensitivity of the patient, and the route of administration.

The practitioner, who will consider the factors related to the individual requiring treatment, will determine the exact dosage. Dosage and administration are adjusted to provide sufficient levels of the active moiety or to maintain the desired effect. Factors, which may be taken into account, include the severity of the individual's disease state, general health of the patient, age, weight, and gender of the patient, diet, time and frequency of administration, drug combination(s), reaction sensitivities, and tolerance/response to therapy. As a general guide, long-acting pharmaceutical compositions may be administered every 3 to 4 days, every week, or once every two weeks, depending on half-life and clearance rate of the particular formulation. Variations in the dosage levels can be adjusted using standard empirical routines for optimization, as is well understood in the art.

Normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors. Similarly, delivery of polynucleotides or polypeptides will be specific to particular cells, conditions, locations, and the like.

In another embodiment of the present invention, antibodies which specifically bind to the HGPRBMY6 polypeptide may be used for the diagnosis of conditions or diseases characterized by expression (or overexpression) of the HGPRBMY6 polynucleotide or polypeptide, or in assays to monitor patients being treated with the HGPRBMY6 polypeptide, or its agonists, antagonists, or inhibitors. The antibodies useful for diagnostic purposes may be prepared in the same manner as those described above for use in therapeutic methods. Diagnostic assays for the HGPRBMY6 polypeptide include methods, which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules, which are known in the art may be used, several of which are described above.

The use of mammalian cell reporter assays to demonstrate functional coupling of known GPCRs (G Protein Coupled Receptors) has been well documented in the literature (Gilman, 1987, Boss et al., 1996; Alam & Cook, 1990; George et al., 1997; Selbie & Hill, 1998; Rees et al., 1999). In fact, reporter assays have been successfully used for identifying novel small molecule agonists or antagonists against GPCRs as a class of drug targets (Zlokarnik et al., 1998; George et al., 1997; Boss et al., 1996; Rees et al, 2001). In such reporter assays, a promoter is regulated as a direct consequence of activation of specific signal transduction cascades following agonist binding to a GPCR (Alam & Cook 1990; Selbie & Hill, 1998; Boss et al., 1996; George et al., 1997; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/11or the promiscuous G protein G alpha 15/16-coupled receptors and MAP kinase reporter genes for use in G alpha i/o coupled receptors (Selbie & Hill, 1998; Boss et al., 1996; George et al., 1997; Blahos, et al., 2001; Offermann & Simon, 1995; Gilman, 1987; Rees et al., 2001). Transcriptional response elements that regulate the expression of Beta-Lactamase within a CHO K1 cell line (CHO-NFAT/CRE: Aurora Biosciences™) (Zlokamik et al., 1998) have been implemented to characterize the function of the orphan HGPRBMY6 polypeptide of the present invention. The system enables demonstration of constitutive G-protein coupling to endogenous cellular signaling components upon intracellular overexpression of orphan receptors. Overexpression has been shown to represent a physiologically relevant event. For example, it has been shown that overexpression occurs in nature during metastatic carcinomas, wherein defective expression of the monocyte chemotactic protein I receptor, CCF2, in macrophages is associated with the incidence of human ovarian carcinoma (Sica, et al., 2000; Salcedo et al., 2000). Indeed, it has been shown that overproduction of the Beta 2 Adrenergic Receptor in transgenic mice leads to constitutive activation of the receptor signaling pathway such that these mice exhibit increased cardiac output (Kypson et al., 1999; Dorn et al., 1999). These are only a few of the many examples demonstrating constitutive activation of GPCRs whereby many of these receptors are likely to be in the active, R*, conformation (J. Wess 1997) (Example 6).

Several assay protocols including ELISA, RIA, and FACS for measuring HGPRBMY6 polypeptide are known in the art and provide a basis for diagnosing altered or abnormal levels of HGPRBMY6 polypeptide expression. Normal or standard values for HGPRBMY6 polypeptide expression are established by combining body fluids or cell extracts taken from normal mammalian subjects, preferably human, with antibody to the HGPRBMY6 polypeptide under conditions suitable for complex formation. The amount of standard complex formation may be quantified by various methods; photometric means are preferred. Quantities of HGPRBMY6 polypeptide expressed in subject sample, control sample, and disease samples from biopsied tissues are compared with the standard values. Deviation between standard and subject values establishes the parameters for diagnosing disease.

### Microarrays and Screening Assays

In another embodiment of the present invention, oligonucleotides, or longer fragments derived from the HGPRBMY6 polynucleotide sequence described herein may be used as targets in a microarray. The microarray can be used to monitor the expression level of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, the nucleic acid sequence, which encodes the HGPRBMY6 polypeptide may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

Fluorescent In Situ Hybridization (FISH), (as described in I. Verma et al., 1988, Human Chromosomes: A Manual of Basic Techniques Pergamon Press, New York, N.Y.) may be correlated with other physical chromosome mapping techniques and genetic map data. Examples of genetic map data can be found in numerous scientific journals or at Online Mendelian Inheritance in Man (OMIM). Correlation between the location of the gene encoding the HGPRBMY6 polypeptide on a physical chromosomal map and a specific disease, or predisposition to a specific disease, may help delimit the region of DNA associated with that genetic disease. The nucleotide sequences, particularly that of SEQ ID NO:2, or fragments thereof, according to this invention may be used to detect differences in gene sequences between normal, carrier, or affected individuals.

In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers, even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (R. A. Gatti et al., 1988, Nature, 336:577-580), any sequences mapping to that area may represent associated or regulatory genes for further investigation. The nucleotide sequence of the present invention may also be used to detect differences in the chromosomal location due to translocation, inversion, and the like, among normal, carrier, or affected individuals.

In another embodiment of the present invention, the HGPRBMY6 polypeptide, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the HGPRBMY6 polypeptide, or portion thereof, and the agent being tested, may be measured utilizing techniques commonly practiced in the art.

Another technique for drug screening which may be used provides for high throughput screening of compounds having suitable binding affinity to the protein of interest as described in WO 84/03564 (Venton, et al.). In this method, as applied to the HGPRBMY6 protein, large numbers of different small test compounds are synthesized on a solid substrate, such as plastic pins or some other surface. The test compounds are reacted with the HGPRBMY6 polypeptide, or fragments thereof, and washed. Bound HGPRBMY6 polypeptide is then detected by methods well known in the art. Purified HGPRBMY6 polypeptide can also be coated directly onto plates for use in the aforementioned drug screening techniques. Alternatively, non-neutralizing antibodies can be used to capture the peptide and immobilize it on a solid support.

In a further embodiment of this invention, competitive drug screening assays can be used in which neutralizing antibodies, capable of binding the HGPRBMY6 polypeptide, specifically compete with a test compound for binding to the HGPRBMY6 polypeptide. In this manner, the antibodies can be used to detect the presence of any peptide, which shares one or more antigenic determinants with the HGPRBMY6 polypeptide.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publication's, for example, Sambrook, Fritsch, and Maniatis, Molecular Cloning: a Laboratory Manual, 2^{nd} Edition, Cold Spring Harbor Laboratory Press, USA, (1989).

### Example 1

### Bioinformatics Analysis

G-protein coupled receptor sequences were used as a probe to search the Incyte and public domain EST databases. The search program used was gapped BLAST (S. F. Altschul, et al., Nuc. Acids Res., 25:3389-4302 (1997)). The top EST hits from the BLAST results were searched back against the non-redundant protein and patent sequence databases. From this analysis, ESTs encoding potential novel GPCRs were identified based on sequence homology. The Incyte EST (CloneID: 2206642) was selected as a potential novel GPCR candidate, called HGPRBMY6, for subsequent analysis. This EST was sequenced and the full-length clone of this GPCR was obtained using the EST sequence information and conventional methods. The complete protein sequence of HGPRBMY6 was analyzed for potential transmembrane domains. The TMPRED program (K. Hofmann and W. Stoffel, Biol. Chem., 347:166 (1993)) was used for transmembrane prediction. The predicted transmembrane (TM) domains of the HGPRBMY6 match with similar predicted domains of related GPCRs at the sequence level. Based on sequence, structure and known GPCR signature sequences, the orphan protein, HGPRBMY6, is a novel human GPCR.

### Example 2

### Cloning of the Novel Human GPCR HGPRBMY6

Using the EST sequence, an antisense 80 base pair oligonucleotide with biotin on the 5′ end was designed to be complementary to the putative coding region of HGPRBMY6 as follows: 5′-b-GCT GTG CAG CGC TGA GTG CGT TCC AGG TAA ATG TCA CTA ACA GAA AAT AGT GCA GTA AGG CGG CAA TCG CAG TGC ACA TG-3′ (SEQ ID NO:5). This biotinylated oligo was incubated with a mixture of single-stranded covalently closed circular cDNA libraries which contained DNA corresponding to the sense strand. Hybrids between the biotinylated oligo and the circular cDNA were captured on streptavidin magnetic beads. Upon thermal release of the cDNA from the biotinylated oligo, the single stranded cDNA was converted into double strands using a primer homologous to a sequence on the cDNA cloning vector. The double stranded cDNA was introduced into E. coli by electroporation and the resulting colonies were screened by PCR, using a primer pair designed from the EST sequence to identify the proper cDNA.

Oligos used to identify the cDNA by PCR were as follows:

| | |
|---|---|
| HGPRBMY6s | |
| 5′-CAGACACCAT TAACATCCCG AAT-3′; and | (SEQ ID NO:6) |
| HGPRBMY6a | |
| 5′-AGAATGAAAT GCCGAGGAAG AG-3′ | (SEQ ID NO:7) |

Those cDNA clones that were positive by PCR had the inserts sized and two of the largest clones (4.2 Kb and 3.5 Kb) were chosen for DNA sequencing. Both clones had identical sequence over the regions in common.

### Example 3

### Expression Profiling of Novel Human GPCR, HGPRBMY6

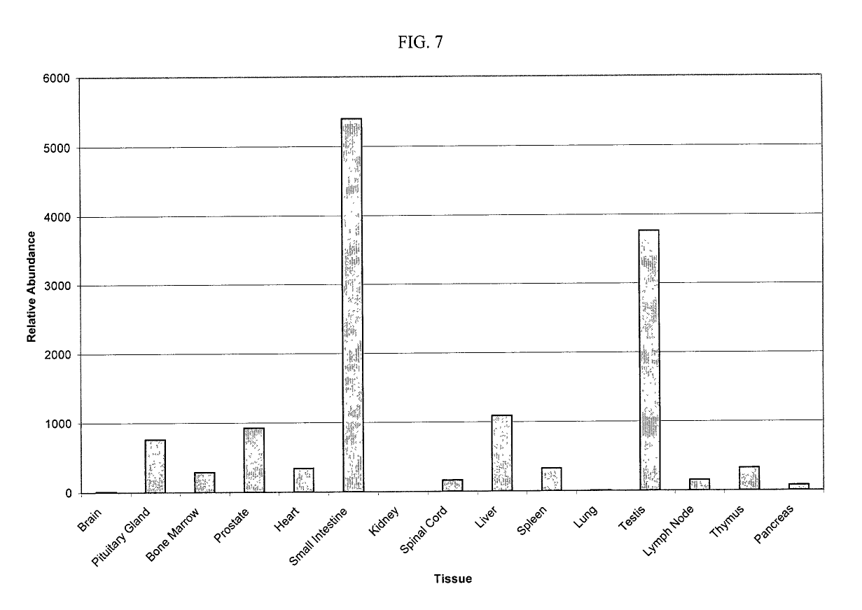

The same PCR primer pair used to identify HGPRBMY6 cDNA clones (HGPRBMY6s-SEQ ID NO:6 and HGPRBMY6a-SEQ ID NO:7) was used to measure the steady state levels of mRNA by quantitative PCR. Briefly, first strand cDNA was made from commercially available mRNA. The relative amount of cDNA used in each assay was determined by performing a parallel experiment using a primer pair for the cyclophilin gene, which is expressed in equal amounts in all tissues. The cyclophilin primer pair detected small variations in the amount of cDNA in each sample, and these data were used for normalization of the data obtained with the primer pair for HGPRBMY6. The PCR data were converted into a relative assessment of the difference in transcript abundance among the tissues tested and the data are presented in FIG. 7. Transcripts corresponding to the orphan GPCR, HGPRBMY6, were found to be highly expressed in small intestine.

### TaqMan Quantitative PCR Analysis of HGPRBMY6

SYBR green quantitative PCR analysis of HGPRBMY6 demonstrated that this GPCR was expressed mainly in the small intestine. Analysis of HGPRBMY6 by TaqMan™ quantitative PCR on an extended panel of tissue RNAs confirms and extends this observation. HGPRBMY6 expression is restricted to the lower small intestine, the ileum and jejunum, where transcripts are found at extremely high levels compared to most other tissues. This evidence supports claims surrounding small molecule modulators of HGPRBMY6 for diseases inflicting the lower digestive tract, such as various types of chronic inflammatory bowel diseases, Crohn's Disease, ulcerative colitis, irritable bowel syndrome, neoplasms of the ileum as well as various disorders of the liver including cirrhosis.

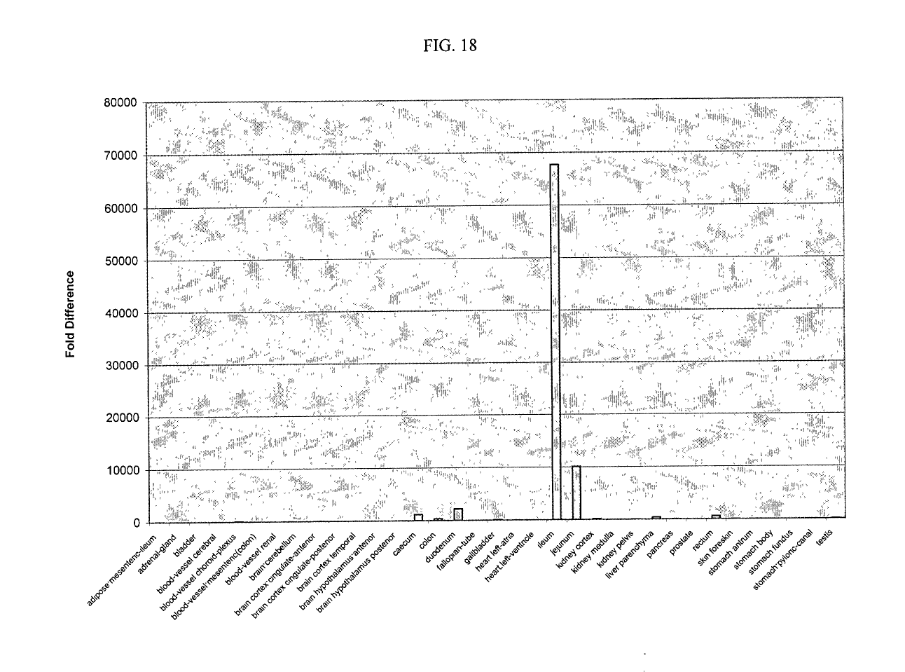

Minor amounts are found in duodenum, caecum and colon, rectum as well as in the liver parenchyma. Expression in the ileum is not associated with mesenteric adipose nor does it appear to be present in the surrounding blood vessels (mesenteric colon). See FIG. 18.

### Example 4

### G-Protein Coupled Receptor PCR Expression Profiling

RNA quantification was performed using the Taqman real-time-PCR fluorogenic assay. The Taqman assay is one of the most precise methods for assaying the concentration of nucleic acid templates.

All cell lines were grown using standard conditions: RPMI 1640 supplemented with 10% fetal bovine serum, 100 IU/ml penicillin, 100 mg/ml streptomycin, and 2 mM L-glutamine, 10 mM Hepes (all from GibcoBRL; Rockville, Md.). Eighty percent confluent cells were washed twice with phosphate-buffered saline (GibcoBRL) and harvested using 0.25% trypsin (GibcoBRL). RNA was prepared using the RNeasy Maxi Kit from Qiagen (Valencia, Calif.).

cDNA template for real-time PCR was generated using the Superscript First Strand Synthesis system for RT-PCR.

SYBR Green real-time PCR reactions were prepared as follows: The reaction mix consisted of 20 ng first strand cDNA; 50 nM Forward Primer; 50 nM Reverse Primer; 0.75×SYBR Green I (Sigma); 1×SYBR Green PCR Buffer (50 mMTris-HCl pH 8.3, 75 mM KCl); 10% DMSO; 3 mM MgCl₂; 300 M each dATP, dGTP, dTTP, dCTP; 1 U Platinum Taq DNA Polymerase High Fidelity (Cat#11304-029; Life Technologies; Rockville, Md.); 1:50 dilution; ROX (Life Technologies). Real-time PCR was performed using an Applied Biosystems 5700 Sequence Detection System. Conditions were 95C for 10 min (denaturation and activation of Platinum Taq DNA Polymerase), 40 cycles of PCR (95C for 15 sec, 60C for 1 min). PCR products are analyzed for uniform melting using an analysis algorithm built into the 5700 Sequence Detection System.

cDNA quantification used in the normalization of template quantity was performed using Taqman technology. Taqman reactions are prepared as follows: The reaction mix consisted of 20 ng first strand cDNA; 25 nM GAPDH-F3, Forward Primer; 250 nM GAPDH-R1 Reverse Primer; 200 nM GAPDH-PVIC Taqman Probe (fluorescent dye labeled oligonucleotide primer); IX Buffer A (Applied Biosystems); 5.5 mM MgCl₂; 300 M dATP, dGTP, dTTP, dCTP; 1 U Amplitaq Gold (Applied Biosystems). GAPDH, D-glyceraldehyde-3-phosphate dehydrogenase, was used as control to normalize mRNA levels.

Real-time PCR was performed using an Applied Biosystems 7700 Sequence Detection System. Conditions were 95C for 10 min. (denaturation and activation of Amplitaq Gold), 40 cycles of PCR (95C for 15 sec, 60C for 1 min).

The sequences for the GAPDH oligonucleotides used in the Taqman reactions are as follows:

| | | |
|---|---|---|
| GAPDH-F3- | 5′-AGCCGAGCCACATCGCT-3′ | (SEQ ID NO:24) |
| GAPDH-R1- | 5′-GTGACCAGGCGCCCAATAC-3′ | (SEQ ID NO:25) |
| GAPDH-PVIC Taqman Probe-VIC- | 5′-CAAATCCGTTGACTCCGACCTTCACCTT-3′ TAMRA. | (SEQ ID NO:26) |

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the Ct equation (Applied Biosystems Prism 7700 Sequence Detection System User Bulletin #2), which is used to calculate a GAPDH normalized relative cDNA level for each specific cDNA. The Ct equation is as follows: relative quantity of nucleic acid template=2^{Ct}=2^{(Cta-Ctb)}, where Cta=Ct target−Ct GAPDH, and Ctb=Ct reference−Ct GAPDH. (No reference cell line was used for the calculation of relative quantity; Ctb was defined as 21).

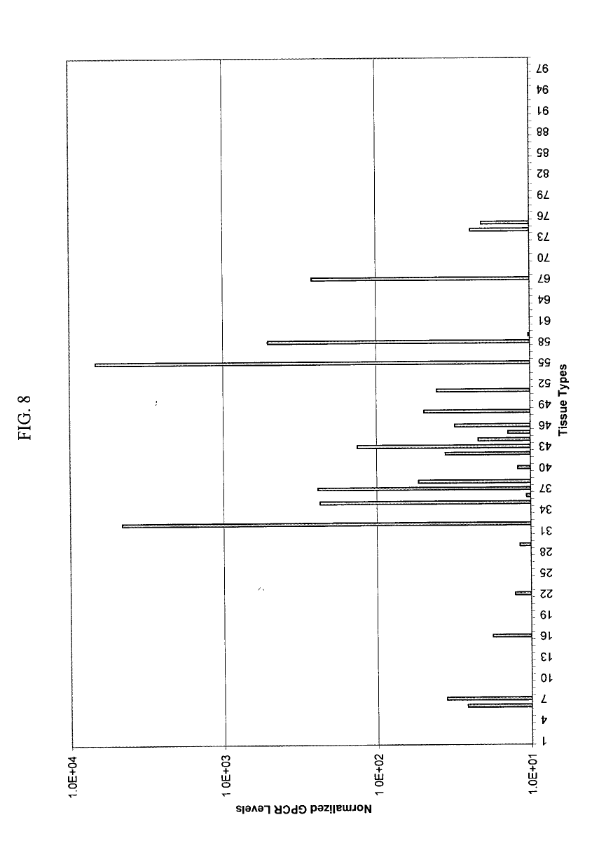

The Graph # of Table 1 corresponds to the tissue type position number of FIG. 8. Interestingly, HGPRBMY6 (also known as GPCR29) messenger RNA was found to be preferentially expressed in colon tumor cell lines. The average colon cell line expresses BMY6 60-fold higher than the average BMY6 expression in non-colon tumor cell lines assayed. Additionally, two of the colon tumor cell lines express BMY6 600 to 800 (579-855)-fold greater than the average expression in non-colon tumor cell lines in the OCLP-1 (oncology cell line panel) assayed.

TABLE 1

| Graph | | | Ct | Ct | | | |
|---|---|---|---|---|---|---|---|
| # | Name | Tissue | GAPDH | GPCR29 | dCt | ddCt | Quant. |
| 1 | AIN 4 | breast | 17.49 | 37.38 | 19.89 | −1.11 | 2.2E+00 |
| 2 | AIN 4T | breast | 17.15 | 38.2 | 21.05 | 0.05 | 9.7E−01 |
| 3 | AIN4/myc | breast | 17.81 | 40 | 22.19 | 1.19 | 0.0E+00 |
| 4 | BT-20 | breast | 17.9 | 38.73 | 20.83 | −0.17 | 1.1E+00 |
| 5 | BT-474 | breast | 17.65 | 40 | 22.35 | 1.35 | 0.0E+00 |
| 6 | BT-483 | breast | 17.45 | 33.75 | 16.3 | −4.7 | 2.6E+01 |
| 7 | BT-549 | breast | 17.55 | 33.4 | 15.85 | −5.15 | 3.6E+01 |
| 8 | DU4475 | breast | 18.1 | 40 | 21.9 | 0.9 | 0.0E+00 |
| 9 | H3396 | breast | 18.04 | 40 | 21.96 | 0.96 | 0.0E+00 |
| 10 | HBL100 | breast | 17.02 | 40 | 22.98 | 1.98 | 0.0E+00 |
| 11 | Her2 MCF-7 | breast | 19.26 | 40 | 20.74 | −0.26 | 0.0E+00 |
| 12 | HS 578T | breast | 17.83 | 36.64 | 18.81 | −2.19 | 4.6E+00 |
| 13 | MCF7 | breast | 17.83 | 40 | 22.17 | 1.17 | 0.0E+00 |
| 14 | MCF-7/AdrR | breast | 17.23 | 36.44 | 19.21 | −1.79 | 3.5E+00 |
| 18 | MDAH 2774 | breast | 16.87 | 38.31 | 21.44 | 0.44 | 7.4E−01 |
| 16 | MDA-MB-175-VII | breast | 15.72 | 32.57 | 16.85 | −4.15 | 1.8E+01 |
| 17 | MDA-MB-231 | breast | 17.62 | 40 | 22.38 | 1.38 | 0.0E+00 |
| 18 | MDA-MB-453 | breast | 17.9 | 36.85 | 18.95 | −2.05 | 4.1E+00 |
| 19 | MDA-MB-468 | breast | 17.49 | 35.95 | 18.46 | −2.54 | 5.8E+00 |
| 20 | Pat-21 R60 | breast | 35.59 | 40 | 4.41 | −16.59 | ND |
| 21 | SKBR3 | breast | 17.12 | 35.66 | 18.54 | −2.46 | 5.5E+00 |
| 22 | T47D | breast | 18.86 | 36.2 | 17.34 | −3.66 | 1.3E+01 |
| 23 | UACC-812 | breast | 17.06 | 36.72 | 19.66 | −1.34 | 2.5E+00 |
| 24 | ZR-75-1 | breast | 15.95 | 40 | 24.05 | 3.05 | 0.0E+00 |
| 25 | C-33A | cervical | 17.49 | 38.24 | 20.75 | −0.25 | 1.2E+00 |
| 26 | Ca Ski | cervical | 17.38 | 40 | 22.62 | 1.62 | 0.0E+00 |
| 27 | HeLa | cervical | 17.59 | 40 | 22.41 | 1.41 | 0.0E+00 |
| 28 | HT-3 | cervical | 17.42 | 36.52 | 19.1 | −1.9 | 3.7E+00 |
| 29 | ME-180 | cervical | 16.86 | 34.31 | 17.45 | −3.55 | 1.2E+01 |
| 30 | SiHa | cervical | 18.07 | 37.96 | 19.89 | −1.11 | 2.2E+00 |
| 31 | SW756 | cervical | 15.59 | 37.48 | 21.89 | 0.89 | 5.4E−01 |
| 32 | CACO-2 | colon | 17.56 | 26.39 | 8.83 | −12.17 | 4.6E+03 |
| 33 | CCD-112Co | colon | 18.03 | 36.73 | 18.7 | −2.3 | 4.9E+00 |
| 34 | CCD-33Co | colon | 17.07 | 40 | 22.93 | 1.93 | 0.0E+00 |
| 35 | Colo 205 | colon | 18.02 | 31.14 | 13.12 | −7.88 | 2.4E+02 |
| 36 | Colo 320DM | colon | 17.01 | 34.6 | 17.59 | −3.41 | 1.1E+01 |
| 37 | Colo201 | colon | 17.89 | 30.97 | 13.08 | −7.92 | 2.4E+02 |
| 38 | Cx-1 | colon | 18.79 | 34.05 | 15.26 | −5.74 | 5.3E+01 |
| 39 | ddH2O | colon | 40 | 40 | 0 | −21 | ND |
| 40 | HCT116 | colon | 17.59 | 35 | 17.41 | −3.59 | 1.2E+01 |
| 41 | HCT116/epo5 | colon | 17.71 | 36.42 | 18.71 | −2.29 | 4.9E+00 |
| 42 | HCT116/ras | colon | 17.18 | 33.03 | 15.85 | −5.15 | 3.6E+01 |
| 43 | HCT116/TX15CR | colon | 17.36 | 31.3 | 13.94 | −7.06 | 1.3E+02 |
| 44 | HCT116/vivo | colon | 17.7 | 34.26 | 16.56 | −4.44 | 2.2E+01 |
| 45 | HCT116/VM46 | colon | 17.87 | 35.07 | 17.2 | −3.8 | 1.4E+01 |
| 46 | HCT116/VP35 | colon | 17.3 | 33.35 | 16.05 | −4.95 | 3.1E+01 |
| 47 | HCT-8 | colon | 17.44 | 40 | 22.56 | 1.56 | 0.0E+00 |
| 48 | HT-29 | colon | 17.9 | 33.29 | 15.39 | −5.61 | 4.9E+01 |
| 49 | LoVo | colon | 17.64 | 40 | 22.36 | 1.36 | 0.0E+00 |
| 50 | LS 174T | colon | 17.93 | 36.1 | 18.17 | −2.83 | 7.1E+00 |
| 51 | LS123 | colon | 17.65 | 33.31 | 15.66 | −5.34 | 4.1E+01 |
| 52 | MIP | colon | 16.92 | 40 | 23.08 | 2.08 | 0.0E+00 |
| 53 | SK-CO-1 | colon | 17.75 | 35.52 | 17.77 | −3.23 | 9.4E+00 |
| 54 | SW1417 | colon | 17.22 | 38.81 | 21.59 | 0.59 | 6.6E−01 |
| 55 | SW403 | colon | 18.39 | 26.66 | 8.27 | −12.73 | 6.8E+03 |
| 56 | SW480 | colon | 17 | 37.82 | 20.82 | −0.18 | 1.1E+00 |
| 57 | SW620 | colon | 17.16 | 40 | 22.84 | 1.84 | 0.0E+00 |
| 58 | SW837 | colon | 18.35 | 30.36 | 12.01 | −8.99 | 5.1E+02 |
| 59 | T84 | colon | 16.44 | 34.09 | 17.65 | −3.35 | 1.0E+01 |
| 60 | CCD-18Co | colon, | 17.19 | 38.1 | 20.91 | −0.09 | 1.1E+00 |
| | | fibroblast | | | | | |
| 61 | HT-1080 | fibrosarcoma | 17.16 | 40 | 22.84 | 1.84 | 0.0E+00 |
| 62 | CCRF-CEM | leukemia | 17.07 | 40 | 22.93 | 1.93 | 0.0E+00 |
| 63 | HL-60 | leukemia | 17.54 | 40 | 22.46 | 1.46 | 0.0E+00 |
| 64 | K562 | leukemia | 18.42 | 36.16 | 17.74 | −3.26 | 9.6E+00 |
| 65 | A-427 | lung | 18 | 40 | 22 | 1 | 0.0E+00 |
| 66 | A549 | lung | 17.63 | 40 | 22.37 | 1.37 | 0.0E+00 |
| 67 | Calu-3 | lung | 18.09 | 31.06 | 12.97 | −8.03 | 2.6E+02 |
| 68 | Calu-6 | lung | 16.62 | 36.23 | 19.61 | −1.39 | 2.6E+00 |
| 69 | ChaGo-K-1 | lung | 17.79 | 35.76 | 17.97 | −3.03 | 8.2E+00 |
| 70 | DMS 114 | lung | 18.14 | 37.86 | 19.72 | −1.28 | 2.4E+00 |
| 71 | LX-1 | lung | 18.17 | 36.99 | 18.82 | −2.18 | 4.5E+00 |
| 72 | MRC-5 | lung | 17.3 | 37.43 | 20.13 | −0.87 | 1.8E+00 |
| 73 | MSTO-211H | lung | 16.81 | 40 | 23.19 | 2.19 | 0.0E+00 |
| 74 | NCI-H596 | lung | 17.73 | 34.14 | 16.41 | −4.59 | 2.4E+01 |
| 75 | SHP-77 | lung | 18.66 | 35.3 | 16.64 | −4.36 | 2.1E+01 |
| 76 | Sk-LU-1 | lung | 15.81 | 34.13 | 18.32 | −2.68 | 6.4E+00 |
| 77 | SK-MES-1 | lung | 17.1 | 40 | 22.9 | 1.9 | 0.0E+00 |
| 78 | SW1271 | lung | 16.45 | 40 | 23.55 | 2.55 | 0.0E+00 |
| 79 | SW1573 | lung | 17.14 | 37.06 | 19.92 | −1.08 | 2.1E+00 |
| 80 | SW900 | lung | 18.17 | 40 | 21.83 | 0.83 | 0.0E+00 |
| 81 | Hs 294T | melanoma | 17.73 | 38.11 | 20.38 | −0.62 | 1.5E+00 |
| 82 | A2780/DDP-R | ovarian | 21.51 | 40 | 18.49 | −2.51 | 0.0E+00 |
| 83 | A2780/DDP-S | ovarian | 17.89 | 39.67 | 21.78 | 0.78 | 5.8E−01 |
| 84 | A2780/epo5 | ovarian | 17.54 | 35.29 | 17.75 | −3.25 | 9.5E+00 |
| 85 | A2780/TAX-R | ovarian | 18.4 | 37.65 | 19.25 | −1.75 | 3.4E+00 |
| 86 | A2780/TAX-S | ovarian | 17.83 | 36.54 | 18.71 | −2.29 | 4.9E+00 |
| 87 | Caov-3 | ovarian | 15.5 | 40 | 24.5 | 3.5 | 0.0E+00 |
| 88 | ES-2 | ovarian | 17.22 | 37.13 | 19.91 | −1.09 | 2.1E+00 |
| 89 | HOC-76 | ovarian | 34.3 | 40 | 5.7 | −15.3 | ND |
| 90 | OVCAR-3 | ovarian | 17.09 | 40 | 22.91 | 1.91 | 0.0E+00 |
| 91 | PA-1 | ovarian | 17.33 | 36.9 | 19.57 | −1.43 | 2.7E+00 |
| 92 | SW 626 | ovarian | 16.94 | 40 | 23.06 | 2.06 | 0.0E+00 |
| 93 | UPN251 | ovarian | 17.69 | 36.52 | 18.83 | −2.17 | 4.5E+00 |
| 94 | LNCAP | prostate | 18.17 | 40 | 21.83 | 0.83 | 0.0E+00 |
| 95 | PC-3 | prostate | 17.25 | 40 | 22.75 | 1.75 | 0.0E+00 |
| 96 | A431 | squamous | 19.85 | 37.73 | 17.88 | −3.12 | 8.7E+00 |

### Cell Line Expression Profile

An expanded expression profile using Taqman™ quantitative PCR revealed that transcripts are found mainly in the small intestine, but also to a lesser extent in the colon. The SYBR Green quantitative PCR analysis of the expression of this transcript across a panel of tumor cell line RNA samples extends and enhances those findings. The study indicated that cell lines derived from colon tissue have aberrant steady state levels of HGPRBMY6 message. Some lines have normal steady state levels of message (CACO-2), whereas others have low or no message present. The present study results support and enhance the notion that this GPCR might be involved in the etiology of diseases affecting the lower digestive tract, and that modulators of HGPRBMY6 activity might have utility in the treatment of these disorders.

PCR primer pairs were designed to the specific gene and used to measure the steady state levels of mRNA by quantitative PCR across a panel of cell line RNA's. Briefly, first strand cDNA was made from several cell line RNA's and subjected to real time quantitative PCR using a PE 7900HT instrument (Applied Biosystems, Foster City, Calif.) which detects the amount of DNA amplified during each cycle by the fluorescent output of SYBR green, a DNA binding dye specific for double stranded DNA. The specificity of the primer pairs for their targets is verified by performing a thermal denaturation profile at the end of the run which gives an indication of the number of different DNA sequences present by determining melting temperature of double stranded amplicon(s). In the experiment, only one DNA fragment of the correct Tm was detected, having a homogeneous melting point.

Small variations in the amount of cDNA used in each tube was determined by performing parallel experiments using a primer pair for a gene expressed in equal amounts in all tissues, cyclophilin. These data were used to normalize the data obtained with the gene specific primer pairs. The PCR data was converted into a relative assessment of the difference in transcript abundance amongst the tissues tested and the data are presented in bar graph form for each transcript.

The formula for calculating the relative abundance is:

Relative abundance=2^{-ΔΔCt}

Where ΔΔCt=(The Ct of the sample−the Ct for cyclophilin)−the Ct for a calibrator sample

The calibrator sample is arbitrarily chosen as the one with the lowest abundance.

For each PCR reaction 10 μL of 2×SybrGreen Master Mix (PE Biosystems) was combined with 4.9 μL water, 0.05 μL of each PCR primer (at 100 μM concentration) and 5 μL of template DNA. The PCR reactions used the following conditions:

95° C. for 10 minutes, then 40 cycles of

95° C. for 30 seconds followed by 60° C. for 1 minute

then the thermal denaturation protocol was begun at 60° C. and the fluorescence measured as the temperature increased slowly to 95° C.

The sequence of the PCR primers was:

| | |
|---|---|
| GPCR-29 sense | |
| CAGACACCATTAACATCCCGAAT | (SEQ ID NO:22) |
| GPCR-29 antisense | |
| AGAATGAAATGCCGAGGAAGAG | (SEQ ID NO:23) |

Representative data is shown below in Table 2, while the graphical analysis is illustrated in FIG. 18.

TABLE 2

| Number | Cell Line | Tissue | Fold change vs. control |
|---|---|---|---|
| 1. | AIN4 | breast | 8.08 |
| 2. | AIN4/myc | breast | 3.43 |
| 3. | AIN4T | breast | 2.37 |
| 4. | BT-20 | breast | 3.71 |
| 5. | BT-474 | breast | 6.32 |
| 6. | BT-549 | breast | 3.10 |
| 7. | DU4475 | breast | 6.46 |
| 8. | H3396 | breast | 76.71 |
| 9. | HBL100 | breast | 39.03 |
| 10. | MCF7 | breast | 10.80 |
| 11. | MCF-7/AdrR | breast | 5.77 |
| 12. | MCF7/Her2 | breast | 10.99 |
| 13. | MDA-MB-175-VII | breast | 2.47 |
| 14. | MDA-MB-231 | breast | 6.71 |
| 15. | C-33A | cervical | 2.64 |
| 16. | Ca Ski | cervical | 35.50 |
| 17. | HeLa | cervical | 4.82 |
| 18. | HT-3 | cervical | 4.05 |
| 19. | ME-180 | cervical | 10.01 |
| 20. | SiHa | cervical | 7.56 |
| 21. | SW756 | cervical | 2.10 |
| 22. | CACO-2 | colon | 7627.01 |
| 23. | Colo201 | colon | 518.40 |
| 24. | HCT116 | colon | 13.08 |
| 25. | HCT116/epo5 | colon | 2.82 |
| 26. | HCT116/ras | colon | 8.01 |
| 27. | HCT116/TX15CR | colon | 278.23 |
| 28. | HCT116/vivo | colon | 10.72 |
| 29. | HCT116/VM46 | colon | 48.72 |
| 30. | HCT116/VP35 | colon | 4.38 |
| 31. | HT-29 | colon | 156.51 |
| 32. | LoVo | colon | 25.07 |
| 33. | LS 174T | colon | 4.38 |
| 34. | 5K-CO-1 | colon | 7.94 |
| 35. | SW480 | colon | 4.07 |
| 36. | SW620 | colon | 4.38 |
| 37. | HUVEC | endothelial | 19.96 |
| 38. | NCI-N87 | gastric | 24.59 |
| 39. | CCRF-CEM | leukemia | 3.78 |
| 40. | HL-60 | leukemia | 6.38 |
| 41. | Jurkat | leukemia | 19.23 |
| 42. | K-562 | leukemia | 10.88 |
| 43. | A-427 | lung | 1.31 |
| 44. | A549 | lung | 3.37 |
| 45. | Calu-3 | lung | 511.81 |
| 46. | Calu-6 | lung | 1.00 |
| 47. | ChaGo-K-1 | lung | 5.35 |
| 48. | DMS 114 | lung | 9.57 |
| 49. | LX-1 | lung | 6.85 |
| 50. | SHP-77 | lung | 9.58 |
| 51. | Sk-LU-1 | lung | 15.66 |
| 52. | SK-MES-1 | lung | 4.39 |
| 53. | SW1271 | lung | 4.66 |
| 54. | SW1573 | lung | 5.09 |
| 55. | SW900 | lung | 9.49 |
| 56. | TOTAL RNA, | lung fetal | 34.21 |
| | FETAL LUNG | | |
| 57. | A-375 | melanoma | 10.62 |
| 58. | C32 | melanoma | 4.66 |
| 59. | G-361 | melanoma | 8.37 |
| 60. | Hs 294T | melanoma | 43.96 |
| 61. | SK-MEL-1 | melanoma | 16.19 |
| 62. | SK-MEL-28 | melanoma | 56.72 |
| 63. | SK-MEL-3 | melanoma | 4.02 |
| 64. | SK-MEL-5 | melanoma | 4.50 |
| 65. | WM373 | melanoma | 42.46 |
| 66. | WM852 | melanoma | 5.97 |
| 67. | A2780/DDP-R | ovarian | 3.29 |
| 68. | A2780/DDP-S | ovarian | 2.93 |
| 69. | A2780/epo5 | ovarian | 5.52 |
| 70. | A2780/TAX-R | ovarian | 6.39 |
| 71. | A2780/TAX-S | ovarian | 56.33 |
| 72. | Caov-3 | ovarian | 4.06 |
| 73. | ES-2 | ovarian | 2.17 |
| 74. | HOC-76 | ovarian | 7.84 |
| 75. | OVCAR-3 | ovarian | 7.20 |
| 76. | PA-1 | ovarian | 21.48 |
| 77. | SW626 | ovarian | 3.54 |
| 78. | TOTAL RNA, | ovarian | 626.48 |
| | OVARY | | |
| 79. | 22Rv1 | prostate | 15.87 |
| 80. | CA-HPV-10 | prostate | 8.18 |
| 81. | DU 145 | prostate | 6.67 |
| 82. | LNCAP | prostate | 7.13 |
| 83. | LNCaP-FGC | prostate | 3.19 |
| 84. | PC-3 | prostate | 4.84 |
| 85. | PWR-IE | prostate | 9.10 |
| 86. | RWPE- I | prostate | 82.31 |
| 87. | RWPE-2 | prostate | 4.26 |
| 88. | RPMI-2650 | SCC | 14.20 |
| 89. | SCC-15 | SCC | 18.41 |
| 90. | SCC-25 | SCC | 44.76 |
| 91. | SCC-4 | SCC | 146.14 |
| 92. | SCC-9 | SCC | 131.93 |
| 93. | HS804.SK | skin | 13.58 |
| 94. | A-431 | squamous | 47.56 |

### Example 5

### Signal Transduction Assays

The activity of GPCRs or homologues thereof, can be measured using any assay suitable for the measurement of the activity of a G protein-coupled receptor, as commonly known in the art. Signal transduction activity of a G protein-coupled receptor can be monitor by monitoring intracellular Ca²⁺, cAMP, inositol 1,4,5-triphosphate (IP₃), or 1,2-diacylglycerol (DAG). Assays for the measurement of intracellular Ca²⁺ are described in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the media of the host cells expressing GPCRs. Once inside the cell, the fura-2 ester is hydrolyzed by cytosolic esterases to its non-lipophilic form, and then the dye cannot diffuse back out of the cell. The non-lipophilic form of fura-2 will fluoresce when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a G protein-coupled receptor, the rise of free cytosolic Ca²⁺ concentrations is preceded by the hydrolysis of phosphatidylinositol 4,5-bisphosphate. Hydrolysis of this phospholipid by the phospholipase C yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure the IP₃ concentrations, radioactivity labeled ³H-inositol is added to the media of host cells expressing GPCRs. The ³H-inositol is taken up by the cells and incorporated into IP₃. The resulting inositol triphosphate is separated from the mono and di-phosphate forms and measured (Sakurai et al., EP 480 381). Alternatively, Amersham provides an inositol 1,4,5-triphosphate assay system. With this system Amersham provides tritylated inositol 1,4,5-triphosphate and a receptor capable of distinguishing the radioactive inositol from other inositol phosphates. With these reagents an effective and accurate competition assay can be performed to determine the inositol triphosphate levels.

Cyclic AMP levels can be measured according to the methods described in Gilman et al., Proc. Natl. Acad. Sci. 67:305-312 (1970). In addition, a kit for assaying levels of cAMP is available from Diagnostic Products Corp. (Los Angeles, Calif.).

### Example 5

### GPCR Activity

Another method for screening compounds which are antagonists, and thus inhibit activation of the receptor polypeptide of the present invention is provided. This involves determining inhibition of binding of labeled ligand, such as dATP, dAMP, or UTP, to cells which have the receptor on the surface thereof, or cell membranes containing the receptor. Such a method further involves transfecting a eukaryotic cell with DNA encoding the GPCR polypeptide such that the cell expresses the receptor on its surface. The cell is then contacted with a potential antagonist in the presence of a labeled form of a ligand, such as dATP, dAMP, or UTP. The ligand can be labeled, e.g., by radioactivity, fluorescence, or any detectable label commonly known in the art. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called a binding assay. Naturally, this same technique can be used to determine agonists.

In a further screening procedure, mammalian cells, for example, but not limited to, CHO, HEK 293, Xenopus Oocytes, RBL-2H3, etc., which are transfected, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

In yet another screening procedure, mammalian cells are transfected to express the receptor of interest, and are also transfected with a reporter gene construct that is coupled to activation of the receptor (for example, but not limited to luciferase or beta-galactosidase behind an appropriate promoter). The cells are contacted with a test substance and the receptor agonist (ligand), such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for antagonists or agonists involves introducing RNA encoding the GPCR polypeptide into cells (or CHO, HEK 293, RBL-2H3, etc.) to transiently or stably express the receptor. The receptor cells are then contacted with the receptor ligand, such as dATP, dAMP, or UTP, and a compound to be screened. Inhibition or activation of the receptor is then determined by detection of a signal, such as, cAMP, calcium, proton, or other ions.

### Example 6

### Functional Characterization of HGPRBMY6

The putative GPCR HGPRBMY6 cDNA was PCR amplified using PFU™ (Stratagene). The primers used in the PCR reaction were specific to the HGPRBMY6 polynucleotide and were ordered from Gibco BRL (5 prime primer: 5′-CGGGATGCCTAGATGCTTTCCTTTGCATTGTCACTTTC-3′ (SEQ ID NO:20). The following 3 prime primer was used to add a Flag-tag epitope to the HGPRBMY2 polypeptide for immunocytochemistry: 5′-CGGGGATCCCTACTTGTCGTCGTCGTCCTTGTAGTCCATGATGCTTTCCTTT GCATTGTCACTTTC-3′(SEQ ID NO:21). The product from the PCR reaction was isolated from a 0.8% Agarose gel (Invitrogen) and purified using a Gel Extraction Kit™ from Qiagen.

The purified product was then digested overnight along with the pcDNA3.1 Hygro™ mammalian expression vector from Invitrogen using the HindIII and BamHI restriction enzymes (New England Biolabs). These digested products were then purified using the Gel Extraction Kit™ from Qiagen and subsequently ligated to the pcDNA3.1 Hygro™ expression vector using a DNA molar ratio of 4 parts insert: 1 vector. All DNA modification enzymes were purchased from NEB. The ligation was incubated overnight at 16 degrees Celsius, after which time, one microliter of the mix was used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones were isolated using the Wizard DNA Miniprep System™ from Promega. Positive clones were then confirmed and scaled up for purification using the Qiagen Maxiprep™ plasmid DNA purification kit.

### Cell Line Generation

The pcDNA3.1 hygro vector containing the orphan HGPRBMY6 cDNA was used to transfect CHO-NFAT/CRE (Aurora Biosciences) cells using Lipofectamine 2000™ according to the manufacturers specifications (Gibco BRL). Two days later, the cells were split 1:3 into selective media (DMEM 11056, 600 μg/ml Hygromycin, 200 μg/ml Zeocin, 10% FBS). All cell culture reagents were purchased from Gibco BRL-Invitrogen.

The CHO-NFAT/CRE and the CHO-NFAT G alpha 15 cell lines, transiently or stably transfected with the orphan HGPRBMY6 GPCR, were analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the orphan HGPRBMY6 GPCR, was examined by analyzing the fluorescence emission of the transformed cells at 447 nm and 518 nm. The changes in gene expression were visualized using Beta-Lactamase as a reporter, that, when induced by the appropriate signaling cascade, hydrolyzed an intracellularly loaded, membrane-permeant ester substrate, Cephalosporin-Coumarin-Fluorescein2/Acetoxymethyl (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme was readily apparent since each enzyme molecule produced was capable of changing the fluorescence of many CCF2/AM™ substrate molecules. A schematic of this cell based system is shown below.

[image]

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only—thus giving rise to blue fluorescent emission at 447 nm.

Fluorescent emissions were detected using a Nikon-TE300 microscope equipped with an excitation filter (D405/10X-25), dichroic reflector (430DCLP), and a barrier filter for dual DAPI/FITC (510 nM) to visually capture changes in Beta-Lactamase expression. The FACS Vantage SE was equipped with a Coherent Enterprise II Argon Laser and a Coherent 302C Krypton laser. In flow cytometry, UV excitation at 351-364 nm from the Argon Laser or violet excitation at 407 nm from the Krypton laser were used. The optical filters on the FACS Vantage SE were HQ460/50m and HQ535/40m bandpass separated by a 490 dichroic mirror.

Prior to analyzing the fluorescent emissions from the cell lines as described above, the cells were loaded with the CCF2/AM substrate. A 6×CCF2/AM loading buffer was prepared whereby 1 mM CCF2/AM (Aurora Biosciences) was dissolved in 100% DMSO (Sigma). Stock solution (12 μl) was added to 60 μl of 100 mg/ml Pluronic F127 (Sigma) in DMSO containing 0.1% Acetic Acid (Sigma). This solution was added while vortexing to 1 mL of Sort Buffer (PBS minus calcium and magnesium-Gibco-25 mM HEPES-Gibco-pH 7.4, 0.1% BSA). Cells were placed in serum-free media and the 6×CCF2/AM was added to a final concentration of 1×. The cells were then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998; and BD Biosciences, 1999.

Immunocytochemistry:

The cell lines transfected and selected for expression of Flag-epitope tagged orphan GPCRs were analyzed by immunocytochemistry. The cells were plated at 1×10³ in each well of a glass slide (VWR). The cells were rinsed with PBS followed by acid fixation for 30 minutes at room temperature using a mixture of 5% Glacial Acetic Acid/90% ethanol. The cells were then blocked in 2% BSA and 0.1%Triton in PBS, and incubated for 2 h at room temperature or overnight at 4° C. A monoclonal anti-Flag FITC antibody was diluted at 1:50 in blocking solution and incubated with the cells for 2 h at room temperature. Cells were then washed three times with 0.1% Triton in PBS for five minutes. The slides were overlayed with mounting media dropwise with Biomedia-Gel Mount (Biomedia; Containing Anti-Quenching Agent). Cells were examined at 10×magnification using the Nikon TE300 equipped with FITC filter (535 nm).

There is strong evidence that certain GPCRs exhibit a cDNA concentration-dependent constitutive activity through cAMP response element (CRE) luciferase reporters (Chen et al., 1999). In an effort to demonstrate functional coupling of HGPRBMY6 to known GPCR second messenger pathways, the HGPRBMY6 polypeptide was expressed at high constitutive levels in the CHO-NFAT/CRE cell line. To this end, the HGPRBMY6 cDNA was PCR amplified and subcloned into the pcDNA3.1™ hygro mammalian expression vector as described herein. Early passage CHO-NFAT/CRE cells were then transfected with the resulting pcDNA3.1 hygro™/HGPRBMY6 construct. Transfected and non-transfected CHO-NFAT/CRE cells (control) were loaded with the CCF2 substrate and stimulated with 10 nM PMA, 1 μM Thapsigargin (NFAT stimulator), and 10 μM Forskolin (CRE stimulator) to fully activate the NFAT/CRE element. The cells were then analyzed for fluorescent emission by FACS.

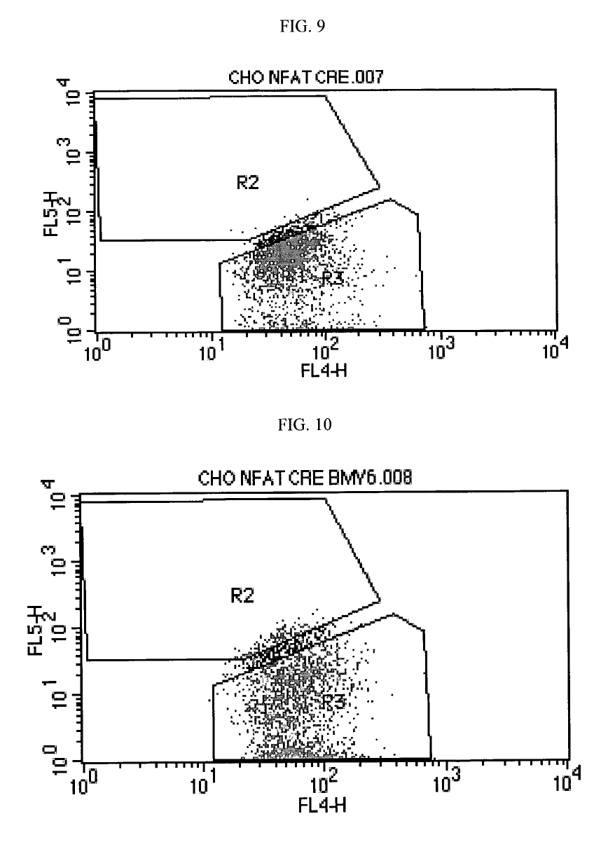

The FACS profile demonstrated the constitutive activity of HGPRBMY6 in the CHO-NFAT/CRE line as evidenced by the significant population of cells with blue fluorescent emission at 447 nm (see FIG. 10: Blue Cells). FIG. 9 describes CHO-NFAT/CRE cell lines transfected with the pcDNA3.1 Hygro™/HGPRBMY6 mammalian expression vector. The cells were then analyzed via FACS according to their wavelength emission at 518 nM (Channel R3—Green Cells), and 447 nM (Channel R2—Blue Cells). As shown, overexpression of HGPRBMY6 resulted in functional coupling and subsequent activation of beta lactamase gene expression, as evidenced by the significant number of cells with fluorescent emission at 447 nM relative to the non-transfected control CHO-NFAT/CRE cells (shown in FIG. 10).

As expected, the NFAT/CRE response element in the untransfected control cell line was not activated (i.e., beta lactamase not induced), enabling the CCF2 substrate to remain intact, and resulting in the green fluorescent emission at 518 nM (see FIG. 9—Green Cells). FIG. 9 describes control CHO-NFAT/CRE (Nuclear Factor Activator of Transcription (NFAT)/cAMP response element (CRE)) cell lines, in the absence of the pcDNA3.1 Hygro™/HGPRBMY6 mammalian expression vector transfection. The cells were analyzed via FACS (Fluorescent Assisted Cell Sorter) according to their wavelength emission at 518 nM (Channel R3−Green Cells), and 447 nM (Channel R2—Blue Cells). As shown, the vast majority of cells emitted at 518 nM, with minimal emission observed at 447 nM. The latter was expected since the NFAT/CRE response elements remain dormant in the absence of an activated G-protein dependent signal transduction pathway (e.g., pathways mediated by Gq/11 or Gs coupled receptors). As a result, the cell permeant, CCF2/AM™ (Aurora Biosciences; Zlokarnik, et al., 1998) substrate remained intact and emitted light at 518 nM. A very low level of leaky Beta Lactamase expression was detectable as evidenced by the small population of cells emitting at 447 nm. Analysis of a stable pool of cells transfected with HGPRBMY6 revealed constitutive coupling of the cell population to the NFAT/CRE response element, activation of Beta Lactamase and cleavage of the substrate (FIG. 10—Blue Cells). These results demonstrated that overexpression of HGPRBMY6 leads to constitutive coupling of signaling pathways known to be mediated by Gq/11 or G alpha 15/16 or Gs coupled receptors that converge to activate either the NFAT or CRE response elements respectively (Boss et al., 1996; Chen et al., 1999).

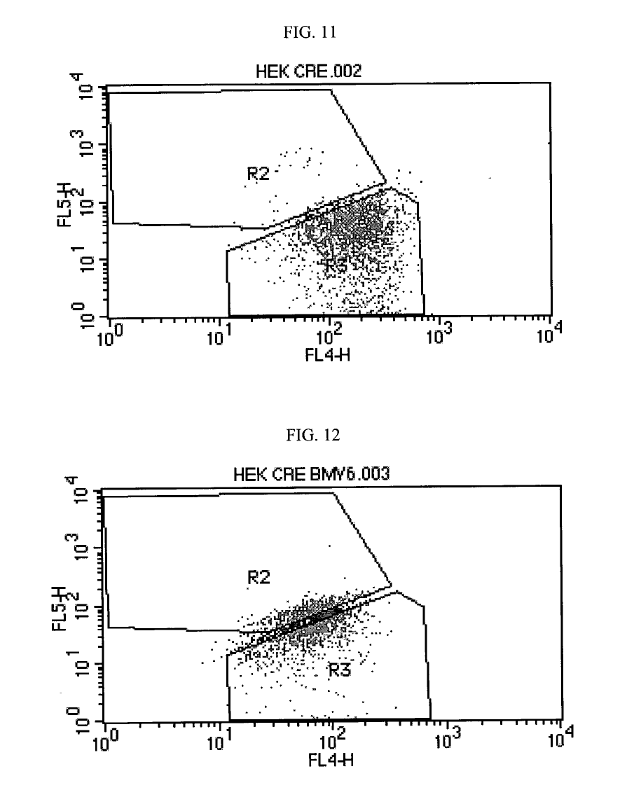

In an effort to further characterize the observed functional coupling of the HGPRBMY6 polypeptide, its ability to couple to the cAMP response element (CRE) independent of the NFAT response element was examined. To this end, the HEK-CRE cell line that contained only the integrated 3XCRE linked to the Beta-Lactamase reporter was transfected with the pcDNA3.1 hygro™/HGPRBMY6 construct. Analysis of the fluorescence emission from this stable pool showed that HGPRBMY6 constitutively coupled to the cAMP mediated second messenger pathways (see FIG. 12 relative to FIG. 11). FIG. 11 describes HEK-CRE cell lines in the absence of the pcDNA3.1 Hygro™/HGPRBMY6 mammalian expression vector transfection. The cells were analyzed via FACS (Fluorescent Assisted Cell Sorter) according to their wavelength emission at 518 nM (Channel R3—Green Cells), and 447 nM (Channel R2—Blue Cells). As shown, the vast majority of cells emitted at 518 nM, with minimal emission observed at 447 nM. The latter was expected since the CRE response elements remain dormant in the absence of an activated G-protein dependent signal transduction pathway (e.g., pathways mediated by Gs coupled receptors). As a result, the cell permeant, CCF2/AM™ (Aurora Biosciences; Zlokarnik, et al., 1998) substrate remained intact and emitted light at 518 nM. FIG. 12 describes HEK-CRE cell lines transfected with the pcDNA3.1 Hygro™/HGPRBMY6 mammalian expression vector analyzed via FACS according to their wavelength emission at 518 nM (Channel R3—Green Cells), and 447 nM (Channel R2—Blue Cells). As shown, overexpression of HGPRBMY6 in the HEK-CRE cells resulted in functional coupling, as evidenced by the insignificant background level of cells with fluorescent emission at 447 nM. Experiments have shown that known Gs coupled receptors demonstrate constitutive activation when overexpressed in the HEK-CRE cell line. For example, direct activation of adenylate cyclase using 10 μM Forskolin has been shown to activate CRE and the subsequent induction of Beta-Lactamase in the HEK-CRE cell line (data not shown). In conclusion, the results were consistent with HGPRBMY6 representing a functional GPCR analogous to known Gs coupled receptors (Boss et al., 1996).

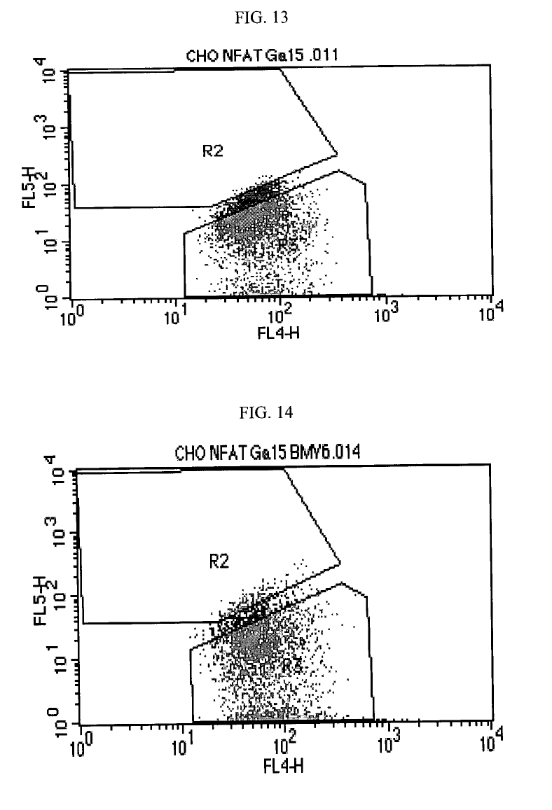

In an effort to further characterize the observed functional coupling of the HGPRBMY6 polypeptide, its ability to couple to a G protein was examined. To this end, the promiscuous G protein, G alpha 15 was utilized. Specific domains of alpha subunits of G proteins have been shown to control coupling to GPCRs (Blahos et al., 2001). It has also been demonstrated that the extreme C-terminal 20 amino acids of either G alpha 15 or 16 confer the unique ability of these G proteins to couple to many GPCRs, including those that naturally do not stimulate PLC (Blahos et al., 2001). Indeed, both G alpha 15 and 16 were shown to couple a wide variety of GPCRs to Phospholipase C activation of calcium mediated signaling pathways (including the NFAT-signaling pathway) (Offermanns & Simon). To demonstrate that HGPRBMY6 was functioning as a GPCR, the CHO-NFAT G alpha 15 cell line that contained only the integrated NFAT response element linked to the Beta-Lactamase reporter was transfected with the pcDNA3.1 hygro™/HGPRBMY6 construct. Analysis of the fluorescence emission from this stable pool showed that HGPRBMY6 constitutively coupled to the NFAT mediated second messenger pathways via G alpha 15 (see FIGS. 13 and 14). FIG. 13 describes control CHO-NFAT G alpha 15 (Nuclear Factor Activator of Transcription (NFAT)) cell lines, in the absence of the pcDNA3.1 Hygro™/HGPRBMY6 mammalian expression vector transfection. The cells were analyzed via FACS (Fluorescent Assisted Cell Sorter) according to their wavelength emission at 518 nM (Channel R3—Green Cells), and 447 nM (Channel R2—Blue Cells). As shown, the vast majority of cells emitted at 518 nM, with minimal emission observed at 447 nM. The latter was expected since the NFAT response elements remained dormant in the absence of an activated G-protein dependent signal transduction pathway (e.g., pathways mediated by G alpha 15 Gq/11 or Gs coupled receptors). As a result, the cell permeant, CCF2/AM™ (Aurora Biosciences; Zlokarnik, et al., 1998) substrate remained intact and emitted light at 518 nM. FIG. 14 describes CHO-NFAT G alpha 15 cell lines transfected with the pcDNA3.1 Hygro™/HGPRBMY6 mammalian expression vector. The cells were analyzed and sorted via FACS according to their wavelength emission at 518 nM (Channel R3—Green Cells), and 447 nM (Channel R2—Blue Cells). As shown, overexpression of HGPRBMY6 resulted in functional coupling and subsequent activation of beta lactamase gene expression, as evidenced by the significant number of cells with fluorescent emission at 447 nM relative to the non-transfected control CHO-NFAT G alpha 15 cells (shown in FIG. 13).

In conclusion, the results were consistent with HGPRBMY6 representing a functional GPCR analogous to known G alpha 15 coupled receptors. Therefore, constitutive expression of HGPRBMY6 in the CHO-NFAT G alpha 15 cell line leads to NFAT activation through accumulation of intracellular Ca²⁺ as has been demonstrated for the M3 muscarinic receptor (Boss et al., 1996). Demonstration of Cellular Expression:

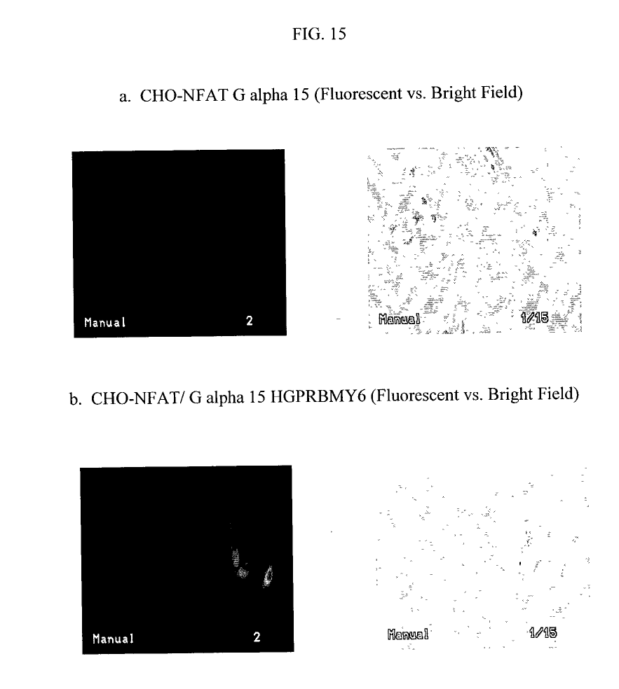

HGPRBMY6 was tagged at the C-terminus using the Flag epitope and inserted into the pcDNA3.1 hygro™ expression vector, as described herein. Immunocytochemistry of CHO-NFAT G alpha 15 cell lines transfected with the Flag-tagged HGPRBMY6 construct with FITC conjugated monoclonal antibody directed against FLAG demonstrated that HGPRBMY6 is indeed a cell surface receptor. The immunocytochemistry also confirmed expression of the HGPRBMY6 in the CHO-NFAT G alpha 15 cell lines. Briefly, CHO-NFAT G alpha 15 cell lines were transfected with pcDNA3.1 hygro™/HGPRBMY6-Flag vector, fixed with 70% methanol, and permeablized with 0.1% TritonX100. The cells were then blocked with 1% Serum and incubated with a FITC conjugated Anti Flag monoclonal antibody at 1:50 dilution in PBS-Triton. The cells were then washed several times with PBS-Triton, overlayed with mounting solution, and fluorescent images were captured (see FIG. 15). FIG. 15 describes CHO-NFAT/CRE cell lines transfected with the pcDNA 3.1 Hygro™/HGPRBMY6-FLAG mammalian expression vector subjected to immunocytochemistry using an FITC conjugated monoclonal antibody against FLAG. Panel A shows the transfected CHO-NFAT/CRE cells under visual wavelengths, and panel B shows the clearly evident fluorescent emission that is consistent with the HGPRBMY6 polypeptide representing a member of the GPCR family. The control cell line, non-transfected CHO-NFAT G alpha 15 cell line, exhibited no detectable background fluorescence (FIG. 15). The HGPRBMY6-FLAG tagged expressing CHO-NFAT G alpha 15 line exhibited specific plasma membrane expression as indicated (FIG. 15).

These data provided clear evidence that HGPRBMY6 was expressed in these cells and the majority of the protein was localized to the cell surface. Cell surface localization was consistent with HGPRBMY6 representing a 7 transmembrane domain containing GPCR. Taken together, the data indicated that HGPRBMY6 was a cell surface GPCR that can function through increases in either cAMP or Ca²⁺ signal transduction pathways via G alpha 15.

### Screening Paradigm

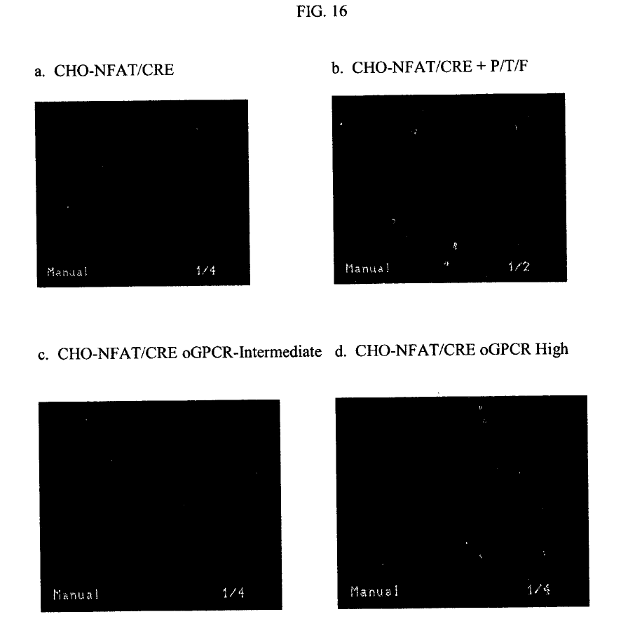

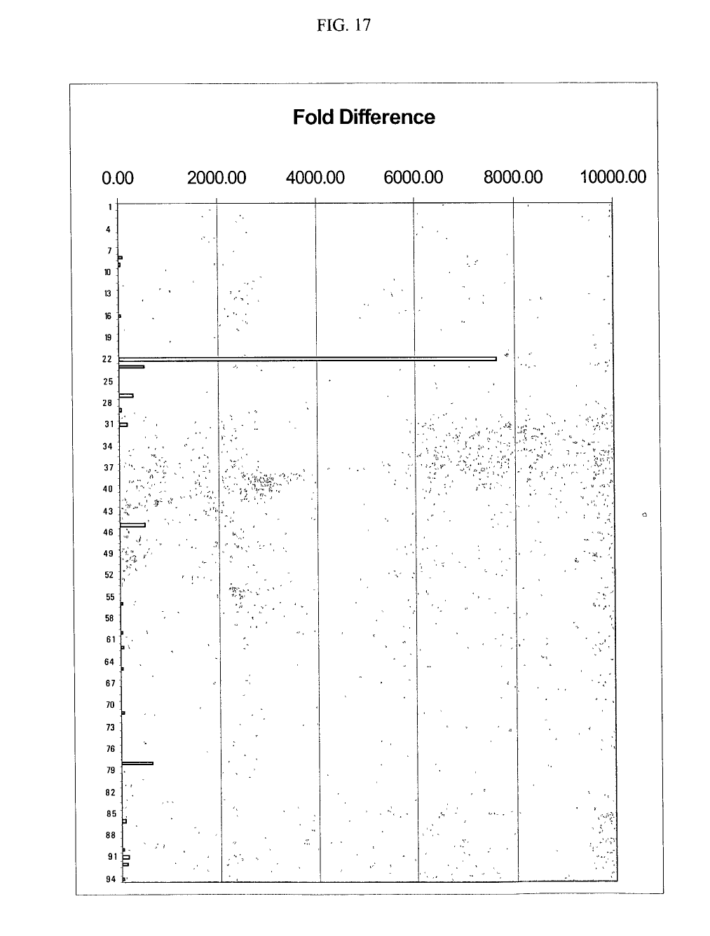

The Aurora Beta-Lactamase technology provided a clear path for identifying agonists and antagonists of the HGPRBMY6 polypeptide. Cell lines that exhibited a range of constitutive coupling activity were identified by sorting through HGPRBMY6 transfected cell lines using the FACS Vantage SE (see FIG. 16). For example, cell lines were sorted that had an intermediate level of orphan GPCR expression, which also correlated with an intermediate coupling response, using the LJL analyst. Such cell lines provided the opportunity to screen, indirectly, for both agonists and antogonists of HGPRBMY6 by searching for inhibitors that block the beta lactamase response, or agonists that increase the beta lactamase response. As described herein, modulating the expression level of beta lactamase directly correlated with the level of cleaved CCF2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca²⁺]i. The data shown represent cell lines that have been engineered with the desired pattern of HGPRBMY6 expression to enable the identification of potent small molecule agonists and antagonists. FIG. 16 describes several CHO-NFAT/CRE cell lines transfected with the pcDNA3.1 Hygro™/HGPRBMY6 mammalian expression vector isolated via FACS that had either intermediate or high beta lactamase expression levels of constitutive activation. Panel A shows untransfected CHO-NFAT/CRE cells prior to stimulation with 10 nM PMA, 1 μM Thapsigargin, and 10 μM Forskolin (−P/T/F) that are representative of the relative background level of beta lactamase expression. Panel B shows CHO-NFAT/CRE cells after stimulation with 10 nM PMA, 1 μM Thapsigargin, and 10 μM Forskolin (+P/T/F), where the cells fully activated the CRE-NFTA response element demonstrating the dynamic range of the assay. Panel C shows a representative orphan GPCR (OGPCR) transfected in CHO-NFAT/CRE cells that had an intermediate level of beta lactamase expression, while panel D shows a representative orphan GPCR transfected in a CHO-NFAT/CRE cell line that had a high level of constitutive beta lactamase expression. HGPRBMY6 modulator screens may be carried out using a variety of high throughput methods known in the art, though preferably using the fully automated Aurora UHTSS system. (FIG. 16; panel a).

### Example 7

### Phage Display Methods for Identifying Peptide Ligands or Modulators of Orphan GPCRS

### Library Construction

Two types of libraries may be created: i.) libraries of 12- and 15 mer peptides for finding peptides that may function as (ant-)agonists and ii.) libraries of peptides with 23-33 random residues that are for finding natural ligands through database searches.

The 15 mer library may be i.) an aliquot of the fUSE5-based 15 mer library originally constructed by G P Smith (Scott, J K and Smith, G P. 1990, Science 249, 386-390). Such a library may be made essentially as described therein, or ii.) a library that is constructed at Bristol-Myers Squibb in vector M13KE (New England Biolabs) using a single-stranded library oligonucleotide extension method (S. S. Sidhu, H. B. Lowman, B. C. Cunningham, J. A. Wells: Methods Enzymol., 2000, vol 328, 333-363).

The 12 mer library is an aliquot of the M13KE-based ‘PhD’ 12 mer library (New England Biolabs).

The libraries with 27-33 random residues are also constructed at Bristol-Myers Squibb in vector M13KE (New England Biolabs) using the method described in (S. S. Sidhu, H. B. Lowman, B. C. Cunningham, J. A. Wells: Methods Enzymol., 2000, vol 328, 333-363).

All libraries in vector M13KE utilize the standard NNK motif to encode the specified number of random residues, where N=A, G, C, or T and where K=G, or T.

The oligos used were:

| | | |
|---|---|---|
| Oligo 1: | 5′-CGAAGCGTAAGGGCCCAGCCGGCC (NNK × 20) CCGGGTCCGGGCGGC-3′ and | (SEQ ID NO:67) |
| Oligo 2: | 5′-AAAAGGAAAAAAGCGGCCGC (VNN × 20) GCCGCCCGGACCCGG-3′, | (SEQ ID NO:68) |

where N = A, G, C, or T and K = C, G, or T and V = C, A, or G.

The oligos were annealed through their 15 base pair complimentary sequences which encode a constant ProGlyProGlyGly (SEQ ID NO:69) pentapeptide sequence between the random 20 amino acid segments, and then extended by standard procedure using Klenow enzyme. This was followed by endonuclease digestion using SfiI and NotI enzymes and ligation to SfiI and NotI cleaved pCantab5E (Pharmacia). The ligation mixture was electroporated into E. coli XL1Blue and phage clones were essentially generated as suggested by the manufacturer for making ScFv antibody libraries in pCantab5E.

### Panning Procedure

Lipofectamine 2000 (Invitrogen)-mediated transfection with DNA encoding oGPCR was used to generate CHO-K1 cells that transiently express oGPCR. After the transfection step, cells were grown at 37° C. for 48 hours in 175 ml flasks in DMEM medium (GIBCO-BRL) supplemented with 10% heat-inactivated fetal bovine serum. Cells were gently shaken off the culture flask or dissociated with trypsin-free dissociation buffer and briefly pelleted, followed by one wash with 20 ml PBS. Cells were then resuspended in PBS+2% milk+protein inhibitor (Roche, Complete™ EDTA-free) and gently agitated at room temperature for 30-60 min to block cells. During this step, phage library (˜1000 copies of each sequence) was preadsorbed with □V□3 integrin (Chemicon; cat#CC1020) that was previously adsorbed to Immulon 4HBX plates in NaHCO₃ buffer (pH 9.6) overnight at 4° C. and then blocked with 2% BSA in PBS. The integrin predasorbtion was followed by a second preadsorbtion step with approximately ˜10⁶˜10⁷ milk-blocked parental CHO-K1 cells. The preadsorbed phage was then added to 10⁶˜10⁷ of the oGPCR-expressing and milk-blocked cells and incubation continued 2 hours at room temperature. This was followed by washing the cells 6×with PBST (PBS+0.2% Tween 20). Elution of remaining phage was with 500 □l 6M urea, pH 3, for 15 min. The eluate was neutralized with 10 ul 2 M Tris-HCL, amplified by standard procedures and the process was repeated for another two panning rounds.

### Sequencing Bound Phage

Standard procedure. Phage in eluates were infected into E. coli host strain (TG1 for 15 mer library; ER2738 for all M13-based libraries) and plates for single colonies. Colonies were grown in liquid and phage in whole culture was sequenced by one of two standard methods with are 1.) with suitable primer generating PCR product of the library segments in the phage genome and then sequencing of the PCR products using one primer of each PCR primer pair or 2.) alternatively, sequencing was carried out after the Templiphi procedure. Sequences were analyzed for conserved sequences both visually and by using the Vector NTI ClustalW alignment tool.

### Peptide Modulators of the Present Invention

The following serve as non-limiting examples of peptides:

| | |
|---|---|
| FAGQIIWYDALDTLM | (SEQ ID NO:70) |
| SDFVGGFWFWDSLFN | (SEQ ID NO:71) |
| GDFWYEACESSCAFW | (SEQ ID NO:72) |
| LEWGSDVFYDVYDCC | (SEQ ID NO:73) |
| RIDSCAKYFLRSCD | (SEQ ID NO:74) |
| CLRSGTGCAFQLYRF | (SEQ ID NO:75) |
| FRVSRVWNPPSFDSA | (SEQ ID NO:76) |
| HAYVECNDTDCRVWF | (SEQ ID NO:77) |
| YYSTPSYADVFRFVW | (SEQ ID NO:90) |
| YFDWTEVPYLMLEEA | (SEQ ID NO:91) |

### Peptide Synthesis

Peptides were synthesized on Fmoc-Knorr amide resin [N-(9-fluorenyl)methoxycarbonyl-Knorr amide-resin; Midwest Biotech; Fishers, Ind.] with an Applied Biosystems (Foster City, Calif.) model 433A synthesizer and the FastMoc chemistry protocol (0.25 mmol scale) supplied with the instrument. Amino acids were double coupled as their N-α-Fmoc-derivatives and reactive side chains were protected as follows: Asp, Glu: t-Butyl ester (OtBu); Ser, Thr, Tyr: t-Butyl ether (tBu); Asn, Cys, Gln, His: Triphenylmethyl (Trt); Lys, Trp: t-Butyloxycarbonyl (Boc); Arg: 2,2,4,6,7-Pentamethyldihydrobenzofuran-5-sulfonyl (Pbf). After the final double coupling cycle, the N-terminal Fmoc group was removed by the multi-step treatment with piperidine in N-Methylpyrrolidone described by the manufacturer. The N-terminal free amines were then treated with 10% acetic anhydride, 5% Diisopropylamine in N-Methylpyrrolidone to yield the N-acetyl-derivative. The protected peptidyl-resins were simultaneously deprotected and removed from the resin by standard methods. The lyophilized peptides were purified on C₁₈ to apparent homogeneity as judged by RP-HPLC analysis. Predicted peptide molecular weights were verified by electrospray mass spectrometry (J. Biol. Chem. 273:12041-12046, 1998).

Cyclic analogs were prepared from the crude linear products. The cysteine disulfide was formed using one of the following methods:

Method 1:

A sample of the crude peptide was dissolved in water at a concentration of 0.5 mg/mL and the pH adjusted to 8.5 with NH₄OH. The reaction was stirred at room temperature, and monitored by RP-HPLC. Once completed, the reaction was adjusted to pH 4 with acetic acid and lyophilized. The product was purified and characterized as above.

Method 2:

A sample of the crude peptide was dissolved at a concentration of 0.5 mg/mL in 5% acetic acid. The pH was adjusted to 6.0 with NH₄OH. DMSO (20% by volume) was added and the reaction was stirred overnight. After analytical RP-HPLC analysis, the reaction was diluted with water and triple lyophilized to remove DMSO. The crude product was purified by preparative RP-HPLC (JACS. 113:6657, 1991)

### Assessing Affect of Peptides on GPCR Function

The effect of any one of these peptides on the function of the GPCR of the present invention may be determined by adding an effective amount of each peptide to each functional assay. Representative functional assays are described more specifically herein, particularly Example 6.

### Uses of the Peptide Modulators of the Present Invention

The aforementioned peptides of the present invention are useful for a variety of purposes, though most notably for modulating the function of the GPCR of the present invention, and potentially with other GPCRs of the same G-protein coupled receptor subclass (e.g., peptide receptors, adrenergic receptors, purinergic receptors, etc.), and/or other subclasses known in the art. For example, the peptide modulators of the present invention may be useful as HGPRBMY6 agonists. Alternatively, the peptide modulators of the present invention may be useful as HGPRBMY6 antagonists of the present invention. In addition, the peptide modulators of the present invention may be useful as competitive inhibitors of the HGPRBMY6 cognate ligand(s), or may be useful as non-competitive inhibitors of the HGPRBMY6 cognate ligand(s).

Furthermore, the peptide modulators of the present invention may be useful in assays designed to either deorphan the HGPRBMY6 polypeptide of the present invention, or to identify other agonists or antagonists of the HGPRBMY6 polypeptide of the present invention, particularly small molecule modulators.

### Example 8

### Method of Creating N- and C-Terminal Deletion Mutants Corresponding to the HGPRBMY6 Polypeptide

As described elsewhere herein, the present invention encompasses the creation of N- and C-terminal deletion mutants, in addition to any combination of N- and C-terminal deletions thereof, corresponding to the HGPRBMY6 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutants of the present invention, exemplary methods are described below.

Briefly, using the isolated cDNA clone encoding the full-length HGPRBMY6 polypeptide sequence, appropriate primers of about 15-25 nucleotides derived from the desired 5′ and 3′ positions of SEQ ID NO: 1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the D198 to I560 N-terminal deletion mutant, the following primers could be used to amplify a cDNA fragment corresponding to this deletion mutant:

| | | |
|---|---|---|
| 5′ | 5′-GCAGCA GCGGCCGC GACATATTATCCAACGTTGGATGTG-3′ | (SEQ ID NO:78) |
| Primer | [symbol] | |
| 3′ | 5′- GCAGCA GTCGAC GATGCTTTCCTTTGCATTGTCAC-3′ | (SEQ ID NO:79) |
| Primer | [symbol] | |

For example, in the case of the M1 to Y483 C-terminal deletion mutant, the following primers could be used to amplify a cDNA fragment corresponding to this deletion mutant:

| | | |
|---|---|---|
| 5′ | 5′-GCAGCA GCGGCCGC ATGGAGACTTATTCCTTGTCTTTGG-3′ | (SEQ ID NO:80) |
| Primer | [symbol] | |
| 3′ | 5′-GCAGCA GTCGAC GTACAGGATAAAAATTTGCAATCCC-3′ | (SEQ ID NO:81) |
| Primer | [symbol] | |

Representative PCR amplification conditions are provided below, although the skilled artisan would appreciate that other conditions may be required for efficient amplification. A 100 ul PCR reaction mixture may be prepared using 10 ng of the template DNA (cDNA clone of HGPRBMY6), 200 uM 4dNTPs, 1 uM primers, 0.25U Taq DNA polymerase (PE), and standard Taq DNA polymerase buffer. Typical PCR cycling condition are as follows:

20-25 cycles: 45 sec, 93 degrees

2 min, 50 degrees

2 min, 72 degrees

1 cycle: 10 min, 72 degrees

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HGPRBMY6 gene (SEQ ID NO:1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HGPRBMY6 gene (SEQ ID NO:1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO: 1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

In preferred embodiments, the following N-terminal HGPRBMY6 deletion polypeptides are encompassed by the present invention: M1-I560, E2-I560, T3-I560, Y4-I560, S5-I560, L6-I560, S7-I560, L8-I560, G9-I560, N10-I560, Q11-I560, S12-I560, V13-I560, V14-I560, E15-I560, P16-I560, N17-I560, I18-I560, A19-I560, I20-I560, Q21-I560, S22-I560, A23-I560, N24-I560, F25-I560, S26-I560, S27-I560, E28-I560, N29-I560, A30-I560, V31-I560, G32-I560, P33-I560, S34-I560, N35-I560, V36-I560, R37-I560, F38-I560, S39-I560, V40-I560, Q41-I560, K42-I560, G43-I560, A44-I560, S45-I560, S46-I560, S47-I560, L48-I560, V49-I560, S50-I560, S51-I560, S52-I560, T53-I560, F54-I560, I55-I560, H56-I560, T57-I560, N58-I560, V59-I560, D60-I560, G61-I560, L62-I560, N63-I560, P64-I560, D65-I560, A66-I560, Q67-I560, T68-I560, E69-I560, L70-I560, Q71-I560, V72-I560, L73-I560, L74-I560, N75-I560, M76-I560, T77-I560, K78-I560, N79-I560, Y80-I560, T81-I560, K82-I560, T83-I560, C84-I560, G85-I560, F86-I560, V87-I560, V88-I560, Y89-I560, Q90-I560, N91-I560, D92-I560, K93-I560, L94-I560, F95-I560, Q96-I560, S97-I560, K98-I560, T99-I560, F100-I560, T101-I560, A102-I560, K103-I560, S104-I560, D105-I560, F106-I560, S107-I560, Q108-I560, K109-I560, I110-I560, I111-I560, S112-I560, S113-I560, K114-I560, T115-I560, D116-I560, E117-I560, N118-I560, E119-I560, Q120-I560, D121-I560, Q122-I560, S123-I560, A124-I560, S125-I560, V126-I560, D127-I560, M128-I560, V129-I560, F130-I560, S131-I560, P132-I560, K133-I560, Y134-I560, N135-I560, Q136-I560, K137-I560, E138-I560, F139-I560, Q140-I560, L141-I560, Y142-I560, S143-I560, Y144-I560, A145-I560, C146-I560, V147-I560, Y148-I560, W149-I560, N150-I560, L151-I560, S152-I560, A153-I560, K154-I560, D155-I560, W156-I560, D157-I560, T158-I560, Y159-I560, G160-I560, C161-I560, Q162-I560, K163-I560, D164-I560, K165-I560, G166-I560, T167-I560, D168-I560, G169-I560, F170-I560, L171-I560, R172-I560, C173-I560, R174-I560, C175-I560, N176-I560, H177-I560, T178-I560, T179-I560, N180-I560, F181-I560, A182-I560, V183-I560, L184-I560, M185-I560, T186-I560, F187-I560, K188-I560, K189-I560, D190-I560, Y191-I560, Q192-I560, Y193-I560, P194-I560, K195-I560, S196-I560, L197-I560, D198-I560, I199-I560, L200-I560, S201-I560, N202-I560, V203-I560, G204-I560, C205-I560, A206-I560, L207-I560, S208-I560, V209-I560, T210-I560, G211-I560, L212-I560, A213-I560, L214-I560, T215-I560, V216-I560, I217-I560, F218-I560, Q219-I560, I220-I560, V221-I560, T222-I560, R223-I560, K224-I560, V225-I560, R226-I560, K227-I560, T228-I560, S229-I560, V230-I560, T231-I560, W232-I560, V233-I560, L234-I560, V235-I560, N236-I560, L237-I560, C238-I560, I239-I560, S240-I560, M241-I560, L242-I560, I243-I560, F244-I560, N245-I560, L246-I560, L247-I560, F248-I560, V249-I560, F250-I560, G251-I560, I252-I560, E253-I560, N254-I560, S255-I560, N256-I560, K257-I560, N258-I560, L259-I560, Q260-I560, T261-I560, S262-I560, D263-I560, G264-I560, D265-I560, I266-I560, N267-I560, N268-I560, I269-I560, D270-I560, F271-I560, D272-I560, N273-I560, N274-I560, D275-I560, I276-I560, P277-I560, R278-I560, T279-I560, D280-I560, T281-I560, I282-I560, N283-I560, I284-I560, P285-I560, N286-I560, P287-I560, M288-I560, C289-I560, T290-I560, A291-I560, I292-I560, A293-I560, A294-I560, L295-I560, L296-I560, H297-I560, Y298-I560, F299-I560, L300-I560, L301-I560, V302-I560, T303-I560, F304-I560, T305-I560, W306-I560, N307-I560, A308-I560, L309-I560, S310-I560, A311-I560, A312-I560, Q313-I560, L314-I560, Y315-I560, Y316-I560, L317-I560, L318-I560, I319-I560, R320-I560, T321-I560, M322-I560, K323-I560, P324-I560, L325-I560, P326-I560, R327-I560, H328-I560, F329-I560, I330-I560, L331-I560, F332-I560, I333-I560, S334-I560, L335-I560, I336-I560, G337-I560, W338-I560, G339-I560, V340-I560, P341-I560, A342-I560, I343-I560, V344-I560, V345-I560, A346-I560, I347-I560, T348-I560, V349-I560, G350-I560, V351-I560, 1352-I560, Y353-I560, S354-I560, Q355-I560, N356-I560, G357-I560, N358-I560, N359-I560, P360-I560, Q361-I560, W362-I560, E363-I560, L364-I560, D365-I560, Y366-I560, R367-I560, Q368-I560, E369-I560, K370-I560, I371-I560, C372-I560, W373-I560, L374-I560, A375-I560, I376-I560, P377-I560, E378-I560, P379-I560, N380-I560, G381-I560, V382-I560, I383-I560, K384-I560, S385-I560, P386-I560, L387-I560, L388-I560, W389-I560, S390-I560, F391-I560, I392-I560, V393-I560, P394-I560, V395-I560, T396-I560, I397-I560, I398-I560, L399-I560, I400-I560, S401-I560, N402-I560, V403-I560, V404-I560, M405-I560, F406-I560, I407-I560, T408-I560, I409-I560, S410-I560, I411-I560, K412-I560, V413-I560, L414-I560, W415-I560, K416-I560, N417-I560, N418-I560, Q419-I560, N420-I560, L421-I560, T422-I560, S423-I560, T424-I560, K425-I560, K426-I560, V427-I560, S428-I560, S429-I560, M430-I560, K431-I560, K432-I560, I433-I560, V434-I560, S435-I560, T436-I560, L437-I560, S438-I560, V439-I560, A440-I560, V441-I560, V442-I560, F443-I560, G444-I560, I445-I560, T446-I560, W447-I560, I448-I560, L449-I560, A450-I560, Y451-I560, L452-I560, M453-I560, L454-I560, V455-I560, N456-I560, D457-I560, D458-I560, S459-I560, I460-I560, R461-I560, I462-I560, V463-I560, F464-I560, S465-I560, Y466-I560, I467-I560, F468-I560, C469-I560, L470-I560, F471-I560, N472-I560, T473-I560, T474-I560, Q475-I560, G476-I560, L477-I560, Q478-I560, I479-I560, F480-I560, I481-I560, L482-I560, Y483-I560, T484-I560, V485-I560, R486-I560, T487-I560, K488-I560, V489-I560, F490-I560, Q491-I560, S492-I560, E493-I560, A494-I560, S495-I560, K496-I560, V497-I560, L498-I560, M499-I560, L500-I560, L501-I560, S502-I560, S503-I560, I504-I560, G505-I560, R506-I560, R507-I560, K508-I560, S509-I560, L510-I560, P511-I560, S512-I560, V513-I560, T514-I560, R515-I560, P516-I560, R517-I560, L518-I560, R519-I560, V520-I560, K521-I560, M522-I560, Y523-I560, N524-I560, F525-I560, L526-I560, R527-I560, S528-I560, L529-I560, P530-I560, T531-I560, L532-I560, H533-I560, E534-I560, R535-I560, F536-I560, R537-I560, L538-I560, L539-I560, E540-I560, T541-I560, S542-I560, P543-I560, S544-I560, T545-I560, E546-I560, E547-I560, 1548-I560, T549-I560, L550-I560, S551-I560, E552-I560, S553-I560, and/or D554-I560 of SEQ ID NO:2. Polynucleotide sequences encoding these polypeptides are also included in SEQ ID NO: 1. The present invention also encompasses the use of these N-terminal HGPRBMY6 deletion polypeptides as immunogenic and/or antigenic epitopes as described elsewhere herein.

In preferred embodiments, the following C-terminal HGPRBMY6 deletion polypeptides are encompassed by the present invention: M1-I560, M1-S559, M1-E558, M1-K557, M1-A556, M1-N555, M1-D554, M1-S553, M1-E552, M1-S551, M1-L550, M1-T549, M1-I548, M1-E547, M1-E546, M1-T545, M1-S544, M1-P543, M1-S542, M1-T541, M1-E540, M1-L539, M1-L538, M1-R537, M1-F536, M1-R535, M1-E534, M1-H533, M1-L532, M1-T531, M1-P530, M1-L529, M1-S528, M1-R527, M1-L526, M1-F525, M1-N524, M1-Y523, M1-M522, M1-K521, M1-V520, M1-R519, M1-L518, M1-R517, M1-P516, M1-R515, M1-T514, M1-V513, M1-S512, M1-P511, M1-L510, M1-S509, M1-K508, M1-R507, M1-R506, M1-G505, M1-I504, M1-S503, M1-S502, M1-L501, M1-L500, M1-M499, M1-L498, M1-V497, M1-K496, M1-S495, M1-A494, M1-E493, M1-S492, M1-Q491, M1-F490, M1-V489, M1-K488, M1-T487, M1-R486, M1-V485, M1-T484, M1-Y483, M1-L482, M1-I481, M1-F480, M1-I479, M1-Q478, M1-L477, M1-G476, M1-Q475, M1-T474, M1-T473, M1-N472, M1-F471, M1-L470, M1-C469, M1-F468, M1-I467, M1-Y466, M1-S465, M1-F464, M1-V463, M1-I462, M1-R461, M1-I460, M1-S459, M1-D458, M1-D457, M1-N456, M1-V455, M1-L454, M1-M453, M1-L452, M1-Y451, M1-A450, M1-L449, M1-I448, M1-W447, M1-T446, M1-I445, M1-G444, M1-F443, M1-V442, M1-V441, M1-A440, M1-V439, M1-S438, M1-L437, M1-T436, M1-S435, M1-V434, M1-I433, M1-K432, M1-K431, M1-M430, M1-S429, M1-S428, M1-V427, M1-K426, M1-K425, M1-T424, M1-S423, M1-T422, M1-L421, M1-N420, M1-Q419, M1-N418, M1-N417, M1-K416, M1-W415, M1-L414, M1-V413, M1-K412, M1-I411, M1-S410, M1-I409, M1-T408, M1-I407, M1-F406, M1-M405, M1-V404, M1-V403, M1-N402, M1-S401, M1-I400, M1-L399, M1-I398, M1-I397, M1-T396, M1-V395, M1-P394, M1-V393, M1-I392, M1-F391, M1-S390, M1-W389, M1-L388, M1-L387, M1-P386, M1-S385, M1-K384, M1-I383, M1-V382, M1-G381, M1-N380, M1-P379, M1-E378, M1-P377, M1-I376, M1-A375, M1-L374, M1-W373, M1-C372, M1-I371, M1-K370, M1-E369, M1-Q368, M1-R367, M1-Y366, M1-D365, M1-L364, M1-E363, M1-W362, M1-Q361, M1-P360, M1-N359, M1-N358, M1-G357, M1-N356, M1-Q355, M1-S354, M1-Y353, M1-I352, M1-V351, M1-G350, M1-V349, M1-T348, M1-I347, M1-A346, M1-V345, M1-V344, M1-I343, M1-A342, M1-P341, M1-V340, M1-G339, M1-W338, M1-G337, M1-I336, M1-L335, M1-S334, M1-I333, M1-F332, M1-L331, M1-I330, M1-F329, M1-H328, M1-R327, M1-P326, M1-L325, M1-P324, M1-K323, M1-M322, M1-T321, M1-R320, M1-I319, M1-L318, M1-L317, M1-Y316, M1-Y315, M1-L314, M1-Q313, M1-A312, M1-A311, M1-S310, M1-L309, M1-A308, M1-N307, M1-W306, M1-T305, M1-F304, M1-T303, M1-V302, M1-L301, M1-L300, M1-F299, M1-Y298, M1-H297, M1-L296, M1-L295, M1-A294, M1-A293, M1-I292, M1-A291, M1-T290, M1-C289, M1-M288, M1-P287, M1-N286, M1-P285, M1-I284, M1-N283, M1-I282, M1-T281, M1-D280, M1-T279, M1-R278, M1-P277, M1-I276, M1-D275, M1-N274, M1-N273, M1-D272, M1-F271, M1-D270, M1-I269, M1-N268, M1-N267, M1-I266, M1-D265, M1-G264, M1-D263, M1-S262, M1-T261, M1-Q260, M1-L259, M1-N258, M1-K257, M1-N256, M1-S255, M1-N254, M1-E253, M1-I252, M1-G251, M1-F250, M1-V249, M1-F248, M1-L247, M1-L246, M1-N245, M1-F244, M1-I243, M1-L242, M1-M241, M1-S240, M1-I239, M1-C238, M1-L237, M1-N236, M1-V235, M1-L234, M1-V233, M1-W232, M1-T231, M1-V230, M1-S229, M1-T228, M1-K227, M1-R226, M1-V225, M1-K224, M1-R223, M1-T222, M1-V221, M1-I220, M1-Q219, M1-F218, M1-I217, M1-V216, M1-T215, M1-L214, M1-A213, M1-L212, M1-G211, M1-T210, M1-V209, M1-S208, M1-L207, M1-A206, M1-C205, M1-G204, M1-V203, M1-N202, M1-S201, M1-L200, M1-I199, M1-D198, M1-L197, M1-S196, M1-K195, M1-P194, M1-Y193, M1-Q192, M1-Y191, M1-D190, M1-K189, M1-K188, M1-F187, M1-T186, M1-M185, M1-L184, M1-V183, M1-A182, M1-F181, M1-N180, M1-T179, M1-T178, M1-H177, M1-N176, M1-C175, M1-R174, M1-C173, M1-R172, M1-L171, M1-F170, M1-G169, M1-D168, M1-T167, M1-G166, M1-K165, M1-D164, M1-K163, M1-Q162, M1-C161, M1-G160, M1-Y159, M1-T158, M1-D157, M1-W156, M1-D155, M1-K154, M1-A153, M1-S152, M1-L151, M1-N150, M1-W149, M1-Y148, M1-V147, M1-C146, M1-A145, M1-Y144, M1-S143, M1-Y142, M1-L141, M1-Q140, M1-F139, M1-E138, M1-K137, M1-Q136, M1-N135, M1-Y134, M1-K133, M1-P132, M1-S131, M1-F130, M1-V129, M1-M128, M1-D127, M1-V126, M1-S125, M1-A124, M1-S123, M1-Q122, M1-D121, M1-Q120, M1-E119, M1-N118, M1-E117, M1-D116, M1-T115, M1-K114, M1-S113, M1-S112, M1-I111, M1-I110, M1-K109, M1-Q108, M1-S107, M1-F106, M1-D105, M1-S104, M1-K103, M1-A102, M1-T100, M1-F100, M1-T99, M1-K98, M1-S97, M1-Q96, M1-F95, M1-L94, M1-K93, M1-D92, M1-N91, M1-Q90, M1-Y89, M1-V88, M1-V87, M1-F86, M1-G85, M1-C84, M1-T83, M1-K82, M1-T81, M1-Y80, M1-N79, M1-K78, M1-T77, M1-M76, M1-N75, M1-L74, M1-L73, M1-V72, M1-Q71, M1-L70, M1-E69, M1-T68, M1-Q67, M1-A66, M1-D65, M1-P64, M1-N63, M1-L62, M1-G61, M1-D60, M1-V59, M1-N58, M1-T57, M1-H56, M1-155, M1-F54, M1-T53, M1-S52, M1-S51, M1-S50, M1-V49, M1-L48, M1-S47, M1-S46, M1-S45, M1-A44, M1-G43, M1-K42, M1-Q41, M1-V40, M1-S39, M1-F38, M1-R37, M1-V36, M1-N35, M1-S34, M1-P33, M1-G32, M1-V31, M1-A30, M1-N29, M1-E28, M1-S27, M1-S26, M1-F25, M1-N24, M1-A23, M1-S22, M1-Q21, M1-I20, M1-A19, M1-I18, M1-N17, M1-P16, M1-E15, M1-V14, M1-V13, M1-S12, M1-Q11, M1-N10, M1-G9, M1-L8, and/or M1-S7 of SEQ ID NO:2. Polynucleotide sequences encoding these polypeptides are also included in SEQ ID NO:1. The present invention also encompasses the use of these C-terminal HGPRBMY6 deletion polypeptides as immunogenic and/or antigenic epitopes as described elsewhere herein.

Alternatively, preferred polypeptides of the present invention may comprise polypeptide sequences corresponding to, for example, internal regions of the HGPRBMY6 polypeptide (e.g., any combination of both N- and C-terminal HGPRBMY6 polypeptide deletions) of SEQ ID NO:2. For example, internal regions could be defined by the equation: amino acid NX to amino acid CX, wherein NX refers to any N-terminal deletion polypeptide amino acid of HGPRBMY6 (SEQ ID NO:2), and where CX refers to any C-terminal deletion polypeptide amino acid of HGPRBMY6 (SEQ ID NO:2). Polynucleotides encoding these polypeptides are also provided. The present invention also encompasses the use of these polypeptides as an immunogenic and/or antigenic epitope as described elsewhere herein.

### Example 9

### Method of Enhancing the Biological Activity or Functional Characteristics Through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as descibed by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (W P C, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments-further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 bp fragments could be eluted from said paper using 1M NaCL, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60 s; 94 C for 30 s, 50-55 C for 30 s, and 72 C for 30 s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30 s, 50 C for 30 s, and 72 C for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g., solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular varient of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

The contents of all patents, patent applications, published PCT applications and articles, books, references, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

### Example 10

### The Use of Antisense or Complemntary Oligonucleotides to Modulate HGPRBMY6

Antisense molecules or nucleic acid sequences complementary to the HGPRBMY6 protein-encoding sequence, or any part thereof, is used to decrease or to inhibit the expression of naturally occurring HGPRBMY6. Although the use of antisense or complementary oligonucleotides comprising about 15 to 35 base-pairs is described, essentially the same procedure is used with smaller or larger nucleic acid sequence fragments. An oligonucleotide based on the coding sequence of HGPRBMY6 protein, as shown in FIGS. 1A-B, or as depicted in SEQ ID NO: 1, for example, is used to inhibit expression of naturally occurring HGPRBMY6. The complementary oligonucleotide is typically designed from the most unique 5′ sequence and is used either to inhibit transcription by preventing promoter binding to the coding sequence, or to inhibit translation by preventing the ribosome from binding to the HGPRBMY6 protein-encoding transcript, among others. However, other regions may also be targeted.

Using an appropriate portion of the signal and 5′ sequence of SEQ ID NO: 1, an effective antisense oligonucleotide includes any of about 15-35 nucleotides spanning the region which translates into the signal or 5′ coding sequence, among other regions, of the polypeptide as shown in FIGS. 1A-B (SEQ ID NO:2). Appropriate oligonucleotides are designed using OLIGO 4.06 software and the HGPRBMY6 protein coding sequence (SEQ ID NO:1). Preferred oligonucleotides are deoxynucleotide, or chimeric deoxynucleotide/ribonucleotide based and are provided below. The oligonucleotides were synthesized using chemistry essentially as described in U.S. Pat. No. 5,849,902; which is hereby incorporated herein by reference in its entirety.

| ID# | Sequence | |
|---|---|---|
| 12669 | GGUCCAGUUACACCAUCAAGAGGCU | (SEQ ID NO:82) |
| 12670 | CAUAGAUACUGCCCUGAGACCCGCA | (SEQ ID NO:83) |
| 12671 | CCUACCCUGCCCAUCUGUGAUUAGG | (SEQ ID NO:84) |
| 12672 | CCCUAGUGUUAGCUCUUCCAUGCAU | (SEQ ID NO:85) |
| 12673 | CCAGGCACAUGAAAGGCUGUACAUA | (SEQ ID NO:86) |

The HGPRBMY6 polypeptide was shown to be involved in the regulation of mammalian cell cycle pathways. Subjecting cells with an effective amount of a pool of all five of the above antisense oligoncleotides resulted in a significant decrease in p21 expression/activity providing convincing evidence that HGPRBMY6 at least regulates the activity and/or expression of p21 either directly, or indirectly. Moreover, the results suggest that HGPRBMY6 is involved in the positive regulation of p21 activity and/or expression, either directly or indirectly. The p21 assay used is described below and was based upon the analysis of p21 activity as a downstream marker for proliferative signal transduction events.

### Transfection of Post-Quiescent A549 Cells With AntiSense Oligonucleotides

Materials needed:

A549 cells maintained in DMEM with high glucose (Gibco-BRL) supplemented with 10% Fetal Bovine Serum, 2 mM L-Glutamine, and 1×penicillin/streptomycin.

Opti-MEM (Gibco-BRL)

Lipofectamine 2000 (Invitrogen)

Antisense oligomers (Sequitur)

Polystyrene tubes.

Tissue culture treated plates.

Quiescent cells were prepared as follows:

Day 0: 300, 000 A549 cells were seeded in a T75 tissue culture flask in 10 ml of A549 media (as specified above), and incubated in at 37° C., 5% CO₂ in a humidified incubator for 48 hours.

Day 2: The T75 flasks were rocked to remove any loosely adherent cells, and the A549 growth media removed and replenished with 10 ml of fresh A549 media. The cells were cultured for six days without changing the media to create a quiescent cell population.

Day 8: Quiescent cells were plated in multi-well format and transfected with antisense oligonucleotides.

A549 cells were transfected according to the following:

1. Trypsinize T75 flask containing quiescent population of A549 cells.

2. Count the cells and seed 24-well plates with 60K quiescent A549 cells per well.

3. Allow the cells to adhere to the tissue culture plate (approximately 4 hours).

4. Transfect the cells with antisense and control oligonucleotides according to the following:

(a) A 10×stock of lipofectamine 2000 (10 ug/ml is 10×) was prepared, and diluted lipid was allowed to stand at RT for 15 minutes.

Stock solution of lipofectamine 2000 was 1 mg/ml.

10×solution for transfection was 10 ug/ml.

To prepare 10×solution, dilute 10 ul of lipofectamine 2000 stock per 1 ml of Opti-MEM (serum free media).

(b) A 10×stock of each oligomer was prepared to be used in the transfection.

Stock solutions of oligomers were at 100 uM in 20 mM HEPES, pH 7.5.

10×concentration of oligomer was 0.25 uM.

To prepare the 10×solutions, dilute 2.5 ul of oligomer per 1 ml of Opti-MEM.

(c) Equal volumes of the 10×lipofectamine 2000 stock and the 10×oligomer solutions were mixed well, and incubated for 15 minutes at RT to allow complexation of the oligomer and lipid. The resulting mixture was 5×.

(d) After the 15 minute complexation, 4 volumes of full growth media was added to the oligomer/lipid complexes (solution was 1×).

(e) The media was aspirated from the cells, and 0.5 ml of the 1×oligomer/lipid complexes added to each well.

(f) The cells were incubated for 16-24 hours at 37 C in a humidified CO₂ incubator.

(g) Cell pellets were harvested for RNA isolation and TaqMan analysis of downstream marker genes.

### TaqMan Reactions

Quantitative RT-PCR analysis was performed on total RNA preps that had been treated with DNaseI or poly A selected RNA. The Dnase treatment may be performed using methods known in the art, though preferably using a Qiagen RNeasy kit to purify the RNA samples, wherein DNAse I treatment is performed on the column.

Briefly, a master mix of reagents was prepared according to the following table:

Dnase I Treatment

| Reagent | Per r'xn (in uL) |
|---|---|
| 10x Buffer | 2.5 |
| Dnase I (1 unit/ul @ | 2 |
| 1 unit per ug sample) | |
| DEPC H₂O | 0.5 |
| RNA sample @ 0.1 | 20 |
| ug/ul | |
| (2-3 ug total) | |
| Total | 25 |

Next, 5 ul of master mix was aliquoted per well of a 96-well PCR reaction plate (PE part #N801-0560). RNA samples were adjusted to 0.1 ug/ul with DEPC treated H₂O (if necessary), and 20 ul was added to the aliquoted master mix for a final reaction volume of 25 ul.

The wells were capped using strip well caps (PE part #N801-0935), placed in a plate, and briefly spun in a plate centrifuge (Beckman) to collect all volume in the bottom of the tubes. Generally, a short spin up to 500 rpm in a Sorvall RT is sufficient.

The plates were incubated at 37° C. for 30 mins. Then, an equal volume of 0.1 mM EDTA in 10 mM Tris was added to each well, and heat inactivated at 70° C. for 5 min. The plates were stored at −80° C. upon completion.

### RT Reaction

A master mix of reagents was prepared according to the following table:

RT reaction

| | RT | | No. RT | |
|---|---|---|---|---|
| Reagent | Per Rx'n (in ul) | | Per Rx'n in ul | |
| 10x RT buffer | 5 | | 2.5 | |
| MgCl₂ | 11 | 5.5 | | |
| DNTP mixture | 10 | 5 | | |
| Random Hexamers | 2.5 | 1.25 | | |
| Rnase inhibitors | 1.25 | 0.625 | | |
| RT enzyme | 1.25 | — | | |
| Total RNA 500 ng | 19.0 | max | 10.125 | max |
| (100 ng no RT) | | | | |
| DEPC H₂O | — | | — | |
| Total | 50 | uL | 25 | uL |

Samples were adjusted to a concentration so that 500 ng of RNA was added to each RT rx'n (10 ng for the no RT). A maximum of 19 ul can be added to the RT rx'n mixture (10.125 ul for the no RT.) Any remaining volume up to the maximum values was filled with DEPC treated H₂O, so that the total reaction volume was 50 ul (RT) or 25 ul (no RT).

On a 96-well PCR reaction plate (PE part #N801-0560), 37.5 ul of master mix was aliquoted (22.5 ul of no RT master mix), and the RNA sample added for a total reaction volume of 50 ul (25 ul, no RT). Control samples were loaded into two or even three different wells in order to have enough template for generation of a standard curve.

The wells were capped using strip well caps (PE part #N801-0935), placed in a plate, and spin briefly in a plate centrifuge (Beckman) to collect all volume in the bottom of the tubes. Generally, a short spin up to 500 rpm in a Sorvall RT is sufficient.

For the RT-PCR reaction, the following thermal profile was used:

25° C. for 10 min

48° C. for 30 min

95° C. for 5 min

4° C. hold (for 1 hour)

Store plate @−20° C. or lower upon completion.

### TaqMan Reaction (Template Comes from RT Plate)

A master mix was prepared according to the following table:

TaqMan reaction (per well)

| Reagent | Per Rx'n (in ul) |
|---|---|
| TaqMan Master Mix | 4.17 |
| 100 uM Probe | .025 |
| (SEQ ID NO:89) | |
| 100 uM Forward | .05 |
| primer (SEQ ID NO:87) | |
| 100 uM Reverse | .05 |
| primer (SEQ ID NO:88) | |
| Template | — |
| DEPC H₂O | 18.21 |
| Total | 22.5 |

The primers used for the RT-PCR reaction is as follows:

P21 primer and probes:

| | |
|---|---|
| Forward Primer: | |
| CTGGAGACTCTCAGGGTCGAA | (SEQ ID NO:87) |
| Reverse Primer: | |
| GCGCTTCCAGGACTGCA | (SEQ ID NO:88) |
| TaqMan Probe: | |
| ACAGATTTCTACCACTCCAAACGCCGG | (SEQ ID NO:89) |

Using a Gilson P-10 repeat pipetter, 22.5 ul of master mix was aliquouted per well of a 96-well optical plate. Then, using P-10 pipetter, 2.5 ul of sample was added to individual wells. Generally, RT samples are run in triplicate with each primer/probe set used, and no RT samples are run once and only with one primer/probe set, often gapdh (or other internal control).

A standard curve is then constructed and loaded onto the plate. The curve has five points plus one no template control (NTC, =DEPC treated H₂O). The curve was made with a high point of 50 ng of sample (twice the amount of RNA in unknowns), and successive samples of 25, 10, 5, and 1 ng. The curve was made from a control sample(s) (see above).

The wells were capped using optical strip well caps (PE part #N801-0935), placed in a plate, and spun in a centrifuge to collect all volume in the bottom of the tubes. Generally, a short spin up to 500 rpm in a Sorvall RT is sufficient.

Plates were loaded onto a PE 5700 sequence detector making sure the plate is aligned properly with the notch in the upper right hand corner. The lid was tightened down and run using the 5700 and 5700 quantitation program and the SYBR probe using the following thermal profile:

50° C. for 2 min

95C for 10 min

and the following for 40 cycles:

95C for 15 sec

60° C. for 1 min

Change the reaction volume to 25 ul.

Once the reaction was complete, a manual threshold of around 0.1 was set to minimize the background signal. Additional information relative to operation of the GeneAmp 5700 machine may be found in reference to the following manuals: “GeneAmp 5700 Sequence Detection System Operator Training CD”; and the “User's Manual for 5700 Sequence Detection System”; available from Perkin-Elmer and hereby incorporated by reference herein in their entirety.

### REFERENCES

1. Rees, S., Brown, S., Stables, J.: “Reporter gene systems for the study of G Protein Coupled Receptor signalling in mammalian cells”. In Milligan G. (ed.): Signal Transduction: A practical approach. Oxford: Oxford University Press, 1999: 171-221.

2. Alam, J., Cook, J. L.: “Reporter Genes: Application to the study of mammalian gene transcription”. Anal. Biochem. 1990; 188: 245-254.

3. Selbie, L. A. and Hill, S. J.: “G protein-coupled receptor cross-talk: The fine-tuning of multiple receptor-signaling pathways”. TiPs. 1998; 19: 87-93.

4. Boss, V., Talpade, D. J., and Murphy, T. J.: “Induction of NFAT mediated transcription by Gq-coupled Receptors in lympoid and non-lymphoid cells”. JBC. 1996; 271: 10429-10432.

5. George, S. E., Bungay, B. J., and Naylor, L. H.: “Functional coupling of endogenous serotonin (5-HT1B) and calcitonin (C1a) receptors in CHO cells to a cyclic AMP-responsive luciferase reporter gene”. J. Neurochem. 1997; 69: 1278-1285.

6. Suto, CM, Igna DM: “Selection of an optimal reporter for cell-based high throughput screening assays”. J. Biomol. Screening. 1997; 2: 7-12.

7. Zlokarnik, G., Negulescu, P. A., Knapp, T. E., More, L., Burres, N., Feng, L., Whitney, M., Roemer, K., and Tsien, R. Y. “Quantitation of transcription and clonal selection of single living cells with a B-Lactamase Reporter”. Science. 1998; 279: 84-88.

8. S. Fiering et. al., Genes Dev. 4, 1823 (1990).

9. J. Karttunen and N. Shastri, PNAS 88, 3972 (1991).

10. Hawes, B. E., Luttrell. L. M., van Biesen, T., and Lefkowitz, R. J. (1996) JBC 271, 12133-12136.

11. Gilman, A. G. (1987) Annul. Rev. Biochem. 56, 615-649.

12. Maniatis et al., Cold Spring Harbor Press, 1989.

13. Salcedo, R., Ponce, M. L., Young, H. A., Wasserman, K., Ward, J. M., Kleinman, H. K., Oppenheim, J. J., Murphy, W. J. “Human endothelial cells express CCF2 and respond to MCP-1: direct role of MCP-1 in angiogenesis and tumor progression”. Blood. 2000; 96 (1): 34-40.

14. Sica, A., Saccani, A., Bottazzi, B., Bernasconi, S., Allavena, P., Gaetano, B., LaRossa, G., Scotton, C., Balkwill F., Mantovani, A. “Defective expression of the monocyte chemotactic protein 1 receptor CCF2 in macrophages associated with human ovarian carcinoma”. J. Immunology. 2000; 164: 733-8.

15. Kypson, A., Hendrickson, S., Akhter, S., Wilson, K., McDonald, P., Lilly, R., Dolber, P., Glower, D., Lefkowitz, R., Koch, W. “Adenovirus-mediated gene transfer of the B2 AR to donor hearts enhances cardiac function”. Gene Therapy. 1999; 6: 1298-304.

16. Dorn, G. W., Tepe, N. M., Lorenz, J. N., Kock, W. J., Ligget, S. B. “Low and high level transgenic expression of B2AR differentially affect cardiac hypertrophy and function in Galpha q-overexpressing mice”. PNAS. 1999; 96: 6400-5.

17. J. Wess. “G protein coupled receptor: molecular mechanisms involved in receptor activation and selectivity of G-protein recognition”. FASEB. 1997; 11:346-354.

18. Whitney, M, Rockenstein, E, Cantin, G., Knapp, T., Zlokarnik, G., Sanders, P., Durick, K., Craig, F. F., and Negulescu, P. A. “A genome-wide functional assay of signal transduction in living mammalian cells”. Nature Biotech. 1998; 16: 1329-1333.

19. BD Biosciences: FACS Vantage SE Training Manual. Part Number 11-11020-00 Rev. A. August 1999.

20. Chen, G., Jaywickreme, C., Way, J., Armour S., Queen K., Watson., C., Ignar, D., Chen, W. J., Kenakin, T. “Constitutive Receptor systems for drug discovery”. J. Pharmacol. Toxicol. Methods 1999; 42: 199-206.

21. Blahos, J., Fischer, T., Brabet, I., Stauffer, D., Rovelli, G., Bockaert, J., and Pin, J.-P. “A novel Site on the G alpha-protein that Rocognized Heptahelical Receptors”. J. Biol. Chem. 2001; 275, No. 5, 3262-69.

22. Offermanns, S. & Simon, M. I. “G alpha 15 and G alpha 16 Couple a Wide Variety of Receptors to Phospholipase C”. J. Biol. Chem. 1995; 270, No. 25, 15175-80.

What is claimed is:

1. An isolated nucleic acid molecule consisting of a polynucleotide having a nucleotide sequence selected from the group consisting of:
(a) a polynucleotide fragment of SEQ ID NO: 1 or a polynucleotide fragment of the cDNA sequence included in ATCC Deposit No:PTA-2677, which is hybridizable to SEQ ID NO: 1;
(b) a polynucleotide encoding a polypeptide fragment of SEQ ID NO:2 or a polypeptide fragment encoded by the cDNA sequence included in ATCC Deposit No:PTA-2677, which is hybridizable to SEQ ID NO: 1;
(c) a polynucleotide encoding a polypeptide domain of SEQ ID NO:2 or a polypeptide domain encoded by the cDNA sequence included in ATCC Deposit No:PTA-2677, which is hybridizable to SEQ ID NO: 1;
(d) a polynucleotide encoding a polypeptide epitope of SEQ ID NO:2 or a polypeptide epitope encoded by the cDNA sequence included in ATCC Deposit No:PTA-2677, which is hybridizable to SEQ ID NO: 1;
(e) a polynucleotide encoding a polypeptide of SEQ ID NO:2 or the cDNA sequence included in ATCC Deposit No:PTA-2677, which is hybridizable to SEQ ID NO:1, having biological activity;
(f) a polynucleotide which is a variant of SEQ ID NO: 1;
(g) a polynucleotide which is an allelic variant of SEQ ID NO: 1;
(h) a polynucleotide which encodes a species homologue of the SEQ ID NO:2;
(i) a polynucleotide which represents the complimentary sequence (antisense) of SEQ ID NO:1;
(j) a polynucleotide corresponding to nucleotides 4 to 1680 of SEQ ID NO:1;
(k) a polynucleotide corresponding to nucleotides 1 to 1680 of SEQ ID NO:1; and
(l) a polynucleotide capable of hybridizing under stringent conditions to any one of the polynucleotides specified in (a)-(k), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a G-protein coupled receptor protein.

3. The isolated nucleic acid molecule of claim 1, wherein the polynucleotide fragment comprises a nucleotide sequence encoding the sequence identified as SEQ ID NO:2 or the polypeptide encoded by the cDNA sequence included in ATCC Deposit No:PTA-2676, which is hybridizable to SEQ ID NO:1.

4. A recombinant vector comprising the isolated nucleic acid molecule of claim 1.

5. A method of making a recombinant host cell comprising the isolated nucleic acid molecule of claim 1.

6. A recombinant host cell produced by the method of claim 5.

7. The recombinant host cell of claim 6 comprising vector sequences.

8. An isolated polypeptide comprising an amino acid sequence at least 95% identical to a sequence selected from the group consisting of:
(a) a polypeptide fragment of SEQ ID NO:2 or the encoded sequence included in ATCC Deposit No:PTA-2677;
(b) a polypeptide fragment of SEQ ID NO:2 or the encoded sequence included in ATCC Deposit No:PTA-2677, having biological activity;
(c) a polypeptide domain of SEQ ID NO:2 or the encoded sequence included in ATCC Deposit No:PTA-2677;
(d) a polypeptide epitope of SEQ ID NO:2 or the encoded sequence included in ATCC Deposit No:PTA-2677;
(e) a full length protein of SEQ ID NO:2 or the encoded sequence included in ATCC Deposit No:PTA-2677;
(f) a variant of SEQ ID NO:2;
(g) an allelic variant of SEQ ID NO:2;
(h) a species homologue of SEQ ID NO:2; and
(i) a polypeptide corresponding to amino acids 2 to 560 of SEQ ID NO:2.

9. An isolated antibody that binds specifically to the isolated polypeptide of claim 8.

10. A recombinant host cell that expresses the isolated polypeptide of claim 8.

11. A method of making an isolated polypeptide comprising:
(a) culturing the recombinant host cell of claim 10 under conditions such that said polypeptide is expressed; and
(b) recovering said polypeptide.

12. A polypeptide produced by claim 1 1.

13. A method for preventing, treating, or ameliorating a medical condition, comprising administering to a mammalian subject a therapeutically effective amount of the polypeptide of claim 8 or a modulator thereof.

14. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

15. A method of diagnosing a pathological condition or a susceptibility to a pathological condition in a subject comprising:
(a) determining the presence or amount of expression of the polypeptide of claim 8 in a biological sample; and
(b) diagnosing a pathological condition or a susceptibility to a pathological condition based on the presence or amount of expression of the polypeptide.

16. A method of treating a disease, disorder, or condition related to the brain, gastrointestinal, or pulmonary system, comprising administering the G-protein coupled receptor polypeptide according to claim 8 or modulator thereof in an amount effective to treat the small intestine-, ileum-, jejunum-, colon-, pulmonary-, lung-related disorders.

17. A method for treating, or ameliorating a medical condition with the polypeptide provided as SEQ ID NO:2, or a modulator thereof, wherein the medical condition is a member of the group consisting of: gastrointestinal disorders, ileum disorders, jejunum disorders, colon disorders, colon cancer, proliferative condition of the colon, proliferative disorder associated with p21 modulation, Crohn's disease, inflammatory bowel disease, sacroiliitis, ileitis, malabsorption disorders, short bowel syndrome, celiac sprue, and small intestinal motility disorders, reproductive disorders, infertility, low sperm counts, male reproductive disorders, disorders associated with male secondary sex characteritics, testicular cancer, pulmonary disorders, lung cancer, and proliferative condition of the lung.

18. A method for treating, or ameliorating a medical condition according to claim 17 wherein the modulator is a member of the group consisting of: a small molecule, a peptide, and an antisense molecule.

19. A method for treating, or ameliorating a medical condition according to claim 18 wherein the modulator is an antagonist.

20. A method for treating, or ameliorating a medical condition according to claim 19 wherein the modulator is an agonist.

21. A method of screening for candidate compounds capable of modulating the activity of a G-protein coupled receptor polypeptide, comprising:
(a) contacting a test compound with a cell or tissue expressing the polypeptide comprising an amino acid sequence as set forth in SEQ ID NO:2; and
(b) selecting as candidate modulating compounds those test compounds that modulate activity of the G-protein coupled receptor polypeptide,
wherein said candidate modulating compounds are useful for the treatment of a disorder.

22. A method of diagnosing a pathological condition or a susceptibility to a pathological condition in a subject comprising the steps of (a) determining the presence or amount of expression of the polypeptide of of SEQ ID NO:2 in a biological sample; (b) and diagnosing a pathological condition or a susceptibility to a pathological condition based on the presence or amount of expression of the polypeptide relative to a control, wherein said condition is a member of the group consisting of colon cancer, proliferative condition of the colon, lung cancer, proliferative condition of the lung, proliferative disorder associated with p21 modulation.

23. The method according to claim 22 wherein said cells are CHO cells.

24. The method according to claim 23 wherein said cells comprise a vector comprising the coding sequence of the beta lactamase gene under the control of NFAT response elements.

25. The method according to claim 24 wherein said cells further comprise a vector comprising the coding sequence of G alpha 15 under conditions wherein G alpha 15 is expressed.

26. The method according to claim 25 wherein said cells express a member of the group consisting of: the polypeptide of claim 8 at low levels, the polypeptide of claim 8 at moderate levels, the polypeptide of claim 8 at high levels, beta lactamase at low levels, beta lactamase at moderate levels, and beta lactamase at high levels.
